(19) United States
(12) Patent Application Publication
(10) Pub. No.: US 2025/0319027 A1
(43) Pub. Date: Oct. 16, 2025

(54) NUTRACEUTICAL PARTICLES

(71) Applicant: VitaKey Inc., Washington, DC (US)

(72) Inventors: Aaron C. Anselmo, Chapel Hill, NC (US); Andrea Stamp, Brookline, MA (US); Stephanie Tomasic, Somerville, MA (US); Joseph Collins, Auckland (NZ); James D. Sievert, Pittsboro, NC (US); Ana Jaklenec, Lexington, MA (US); Robert S. Langer, Newton, MA (US); Catherine B. Reynolds, Mountain Brook, AL (US); Wayne Reynolds, McLean, VA (US); Gwangseong Kim, Lexington, MA (US)

(21) Appl. No.: 18/873,573

(22) PCT Filed: Jun. 30, 2023
(86) PCT No.: PCT/US2023/026766

§ 371 Date: Dec. 10, 2024

Related U.S. Application Data
(60) Provisional application No. 63/357,902, filed on Jul. 1, 2022.
(60) Provisional application No. 63/415,264, filed on Oct. 11, 2022.

(51) Int. Cl. A61K 9/16; A61K 9/50; A61K 31/047

(52) U.S. Cl. CPC A61K 9/1635; A61K 9/1617; A61K 9/1623; A61K 9/1652; A61K 9/1682; A61K 9/5026; A61K 9/5036; A61K 31/047

(57) ABSTRACT

Provided herein are compositions and methods for storage and delivery of a nutraceutical (e.g., lutein, zeaxanthin, vitamin, macronutrient, probiotics) with one or more of the following advantages: 1) low organic solvent content; 2) low water activity; 3) selective release based on pH; 4) improved shelf-life and resistance to degradation; and 5) improved compatibility with other nutraceutical products; 6) stability in an aqueous liquid (e.g., at ambient temperature and/or at boiling temperatures); 7) enhanced protection from light; and 8) tunable properties including size, loading, dose, interactions with the surrounding environment, and release conditions.

### CROSS-REFERENCE TO RELATED APPLICATIONS

This application claims priority to U.S. Provisional Patent Application No. 63/357,902, filed Jul. 1, 2022; and U.S. Provisional Patent Application No. 63/415,264, filed Oct. 11, 2022; the title of each of which is “Nutraceutical Particles,” and the content of each of which is incorporated herein by reference in its entirety.

### BACKGROUND

A nutraceutical product can be included in a food, a supplement, or a supplemented (i.e., fortified) food product intended to confer health benefits. Popular nutraceuticals include micronutrients, macronutrients, and probiotics; for example, proteins, amino acids, fish oil, vitamins, antioxidants (e.g., carotenoids and flavonoids), minerals, prebiotics, etc.

### SUMMARY OF THE INVENTION

Some aspects of the current disclosure provide nutraceutical particle preparations (e.g., nutraceutical compositions comprising particles) and technologies (e.g., methods of preparation, use, etc.) relating thereto. In some embodiments, provided particle preparations are characterized by one or more of the following advantages: 1) low residual solvent content; 2) low water activity; 3) selective release based on pH; 4) improved shelf-life and resistance to degradation; 5) improved compatibility with other components of nutraceutical products and/or compositions that include them (e.g., foods, drinks, or other edible materials), specifically including compatibility with water-active-sensitive agents such as probiotics; 6) stability of particles and payload in an aqueous liquid against heat, light, water, and/or oxidation; 7) stability of particles and payload in, or as, a dry powder against heat, light, water, and/or oxidation; 8) enhanced protection from light, heat, water, and/or oxidation; 9) tunable properties including size, loading, dose, interactions with the surrounding environment, and release conditions, etc.; 10) improved anti-caking, anti-clumping, anti-agglomerating, and/or anti-aggregating functionality at elevated temperatures.

In some embodiments, provided particle preparations (e.g., nutraceutical compositions) achieve one or more advantages such as stability, controlled release, and compatibility with other materials.

In some embodiments provided are particle preparations (e.g., microparticle preparations) that provide one or more of the following advantages: (1) Stability enhancement for payload component (e.g., nutraceutical payload component, nutraceutical, micronutrients, macronutrients, minerals, carotenoids, probiotics, prebiotics, vitamins, or a combination thereof) in water, light, increased temperature, and oxidative environments; (2) Compatibility attributes that permit combination of payload components (e.g., nutraceuticals [e.g., carotenoid compounds, vitamins, etc.], micronutrients, macronutrients, minerals, probiotics, prebiotics etc., or combinations thereof) when with (e.g., by mixture with and/or integration into) complex foods and/or beverages (e.g., milk) and/or ingredients (e.g., non-encapsulated probiotics); (3) They have low residual solvent content; (4) They are characterized by low water activity; (5) Stability in aqueous liquids (e.g., water, milk, etc.), even of payload components that otherwise show low water solubility or are insoluble in water; (6) Size characteristics (e.g., average diameter [e.g., about 5 μm] and/or size distribution features) that, among other things, permit homogenous combination with other materials or components; (6) Rapid release of payload component (e.g., nutraceutical payload component, which may be or comprise one or more nutraceuticals [e.g., carotenoid compounds such as lutein, zeaxanthin, etc., and/or one or more vitamins, such as vitamin D, etc.], micronutrients, macronutrients, minerals, probiotics, prebiotics, or a combination) in acidic conditions (e.g., the stomach), and in many embodiments not in other conditions; (7) Modularity of the technology that allows for control over features such as, for example, microparticle size, shape, loading, and release; (8) anti-caking, anti-clumping, anti-agglomerating and/or anti-aggregating functionality (e.g., when particle preparations are provided in dry form [e.g., dry powder]).

In some cases, provided nutraceutical compositions (e.g., particle preparations) are or comprise particles (e.g., microparticles) that include a matrix component (e.g., a polymer component) and a payload component (e.g., nutraceutical payload component). In some instances, one or more layers of matrix components are present.

In some instances, a matrix component is or comprises a polymer component. In some instances, a polymer component is or comprises a pH-responsive polymer component. In some instances, a polymer component is or comprises a temperature-responsive polymer component. In some instances, one or more layers of payload components are present.

In some instances, a matrix component comprises a biocompatible material. In some instances a biocompatible material is or comprises a sugar, a polysaccharide, a carbohydrate, an oil, a fat, a wax, a protein, or a combination thereof. In some instances, a matrix component comprises a salt and a surfactant (e.g., SDS).

In some instances, a matrix component is or comprises a nutraceutical (e.g., nutraceutical matrix component). In some instances, a nutraceutical is or comprises at least one micronutrient, macronutrient, mineral, antioxidant, probiotic, prebiotic, or a combination thereof; in some particular embodiments, a nutraceutical matrix component is or comprises a carotenoid compound (e.g., lutein, zeaxanthin, etc.) and/or a vitamin (e.g., vitamin D, etc.). In some embodiments, a nutraceutical matrix component is or comprises one or more carotenoid compounds. In some embodiments, a nutraceutical matrix component is or comprises vitamin D.

In some cases, a matrix component further comprises one or more bacterial species.

Some aspects of the present disclosure provide technologies for making and/or characterizing matrix components comprising a polymer component described herein, and/or compositions that include them. In some cases, the method of making polymeric matrices involves using aqueous-based atomization. In some cases, solvent-based atomization is involved. In some embodiments, emulsion-based methods are involved. In some embodiments, extrusion-based methods are involved.

In some embodiments, a payload component (e.g., nutraceutical payload component) is or comprises a nutraceutical. In some cases, a nutraceutical is or comprises at least one micronutrient, macronutrient, mineral, antioxidant, probiotic, prebiotic, or a combination thereof; in some particular embodiments, a nutraceutical payload component is or comprises a carotenoid compound (e.g., lutein, zeaxanthin, etc.) and/or a vitamin (e.g., vitamin D, etc.). In some embodiments, a payload component is or comprises one or more carotenoid compounds. In some embodiments, a payload component is or comprises vitamin D.

Regardless of the shape of particles, a particle “diameter” (i.e., a particle size) is the longest distance from one end of the particle to another end of the particle. In some embodiments, nutraceutical compositions (e.g., particle preparations) are or comprise particles (e.g., polymer microparticles) with a distribution of particle diameters (e.g., Dv(10), Dv(20), Dv(30), Dv(40), Dv(50), Dv(60), Dv(70), Dv(80), Dv(90), Dv(99), etc.). In some embodiments, nutraceutical compositions (e.g., particle preparations) are or comprise particles with a distribution of particle diameters (e.g., Dv(10), Dv(20), Dv(30), Dv(40), Dv(50), Dv(60), Dv(70), Dv(80), Dv(90), Dv(99), etc.) of up to about 3000 μm, up to about 2000 μm, up to about 1000 μm, of up to about 500 μm, up to about 400 μm, up to about 300 μm, up to about 200 μm, up to about 100 μm, up to about 50 μm, up to about 40 μm, up to about 30 μm, up to about 20 μm, up to about 10 μm, up to about 5 μm, or up to about 1 μm.

In some embodiments, nutraceutical compositions (e.g., particle preparations) are or comprise particles with an average diameter (e.g., D[3,2], D[4,3], etc.) of particles in the range of about 1-3000 μm, about 1-2000 μm, about 1-1000 μm, about 1-500 μm, about 1-250 μm, about 1-175 μm, about 1-100 μm, about 1-50 μm, about 1-10 μm, or about 4-6 μm.

In some embodiments, particles (e.g., polymer microparticles) may have any shape or form, for example, having a cross-section shape of a sphere, an oval, a triangle, a square, a hexagon, or an irregular shape. In some embodiments, nutraceutical compositions comprise particles (e.g., microparticles), wherein a majority of particles have a common shape. In some embodiments, nutraceutical compositions are or comprise particles of various such shapes in combination.

In some embodiments, provided particle preparations (e.g., nutraceutical compositions) are characterized by having a layered structure wherein adjacent components in the particle preparations have different chemical structure.

In some embodiments, provided particle preparations (e.g., nutraceutical compositions) are characterized by having multiple polymer components, wherein the particle preparations (e.g., nutraceutical compositions) may be additionally encapsulated with a separate polymer component.

In some particular embodiments of layered particle preparations (e.g., nutraceutical compositions) provided by the present disclosure, a first layer is or comprises a hydrophilic material and/or a water-soluble material and a second layer is or comprises a hydrophobic material and/or a fat soluble material. For example, in some particular embodiments, a fat soluble payload material may be or comprise vitamin B; in some embodiments, such fat soluble payload material may be encapsulated or otherwise dispersed within a hydrophobic polymer (e.g., BMC). In some such embodiments, such hydrophobic material (optionally with a hydrophobic or fat-soluble payload) forms a layer and a hydrophilic payload material (e.g., iron) may be encapsulated or otherwise dispersed with a hydrophilic polymer (e.g., HA) in a different layer or in a core (or vice versa) of relevant particles; in other embodiments, layers are reversed.

In some embodiments, provided particle preparations (e.g., nutraceutical compositions) are characterized by low residual solvent content. In some embodiments, the present disclosure provides technologies for preparing and/or characterizing nutraceutical compositions comprising low residual solvent content.

In some embodiments, provided particle preparations (e.g., nutraceutical compositions) are characterized by low water activity. In some embodiments, the present disclosure provides technologies for preparing and/or characterizing nutraceutical compositions comprising low water activity. This is the first reported instance of low water activity particle preparations for lutein and zeaxanthin.

In some embodiments, the present disclosure provides technologies for preparing and/or characterizing particle preparations (e.g., nutraceutical compositions) comprising low residual solvent content and low water activity. This is the first reported instance of both low solvent content and low water activity particle preparations for lutein and zeaxanthin.

Particle preparations (e.g., nutraceutical compositions) comprising low residual solvent content provides benefits over existing products, among other things because ingestion of residual solvents poses health concerns. Further, methods to remove residual solvents from particle preparations (e.g., nutraceutical compositions) are costly.

In some embodiments, the present disclosure provides technologies for manufacturing provided nutraceutical compositions (e.g., provided particle preparations) that reduce or eliminate toxic organic solvents (thereby minimizing or avoiding risk of neutralizing benefit(s) of taking a particular nutritional supplement) and/or water (thereby minimizing or avoiding risk of oxidation). Thus, the present disclosure provides technologies with a variety of advantages.

In some instances, nutraceutical compositions (e.g., particle preparations comprising a nutraceutical payload) disclosed herein comprise a residual solvent content lower than a predetermined amount. In some cases, the residual solvent is an organic solvent, for example, hexane, ethanol, ethyl acetate, acetone, methylene chloride, methanol, dichloromethane, isopropyl alcohol (i.e., 2-propanol), or any combination thereof. In some cases, the total residual solvent content is lower than 5000 ppm. In some cases, the total residual solvent content is lower than 1000 ppm. In some cases, the total residual solvent content is lower than 100 ppm.

In some instances, the residual solvent is dichloromethane, and the residual dichloromethane content is less than 5 ppm. In some instances, the residual solvent is hexane, and the residual hexane content is less than 50 ppm.

In some instances, the residual solvent is isopropyl alcohol (2-propanol), and the residual isopropyl alcohol (2-propanol) content is less than 50 ppm. In some instances, the residual solvent is ethanol, and the residual ethanol content is less than 50 ppm.

In some instances, the residual solvent is methanol, and the residual methanol content is less than 50 ppm. In some instances, the residual solvent is ethyl acetate, and the residual ethyl acetate content is less than 50 ppm. In some instances, the residual solvent is acetone, and the residual acetone content is less than 50 ppm.

In some embodiments, the present disclosure provides particle preparations (e.g., nutraceutical compositions) with low water activity. Disclosed technologies provide benefit over existing products because high water activity formulations lead to rapid degradation of nutraceuticals.

In some embodiments, the present disclosure provides particle preparations (e.g., nutraceutical compositions) with low water activity. In some instances, provided particle preparations (e.g., nutraceutical compositions) may have a water activity of <0.3, <0.2, or <0.1.

In some embodiments, the present disclosure provides particular insight that identifies the source of a problem associated with certain current nutraceutical compositions (e.g., nutraceutical compositions [e.g., particle preparations] comprising lutein and/or zeaxanthin) in that they often contain high water activity components and therefore have limited utility for combination with certain probiotics, as many probiotics rapidly degrade when exposed to high water activity components. This is the first reported instance of maintaining probiotic stability when combined with lutein and zeaxanthin due to decreasing water activity, both in general and in particle preparations.

In some embodiments, particle preparations (e.g., nutraceutical compositions) with low water activity are particularly useful for combination with probiotics (e.g., probiotics sensitive to loss of colony forming units when exposed to high-water-reactivity agents.) In some embodiments, particle preparations (e.g., nutraceutical compositions) may further comprise a probiotic.

In some embodiments, provided particle preparations (e.g., nutraceutical compositions) may comprise both low residual solvent content and have low water activity.

In some embodiments, the particle preparation includes a microparticle loading from about 45% to about 90%, the preparation further comprising: a first excipient component loading in a range from about 10% to about 50%; and a second excipient loading in a range from about 0% to about 45%.

In some embodiments, the particle preparation is formed by adding at least one of the first excipient component and the second excipient component to the microparticle loading during milling.

In some embodiments, at least about 80%, about 90%, and/or about 95% of the payload component is released within 5 minutes when included in an environment having a pH of less than about 5.

In some embodiments, at most about 20%, about 15%, about 10%, and/or about 5% of the payload component is released after soaking the particle preparation for at least about 2 hours in an environment having a pH of about 7 and a temperature within a range of about 25° C. to about 100° C.

In some embodiments, the particle preparation is effective to protect against degradation (e.g., light-induced degradation) of the payload component for at least about 2 weeks, about 1 month, about 2 months, and/or about 3 months, and wherein the degradation comprises at least one of oxidation, hydrolysis, isomerization, fragmentation, or any combination thereof.

In some embodiments, the pH-responsive polymer component is structured to allow controlled release of the payload component when exposed to an environment with a pH of about 5.0 or lower, wherein the pH-responsive polymer component is structured to be stable when exposed to an environment that includes a pH of about 6.0 or higher, and wherein the pH-responsive polymer component is stable when exposed to temperatures in a range from 1° C. to about 100° C.

In some embodiments, the particle preparation includes at least one of: lutein in a particle loading (w/w %) range from about 2% to about 25.6%; zeaxanthin in a particle loading (w/w %) range from about 2% to about 15%; and vitamin D a particle loading (w/w %) range from about 0.9% to about 10.7%.

In some embodiments, the first excipient component comprises at least one of soy lecithin, sunflower lecithin, maltodextrin 40, dryflo, and fructose, wherein the second excipient component comprises at least one of soy lecithin and maltodextrin 40.

In some embodiments, the particle preparation includes a third excipient component loading in a range from about 0% to about 5%, wherein the third excipient comprises dryflo.

In some embodiments, the particle preparation comprises the microparticle loading in a range from about 70% to about 90%, wherein the particle preparation comprises the first excipient component loading in a range from about 10% to about 30%, and wherein the particle preparation comprises the second excipient component loading in a range from about 0% to about 15%.

In some embodiments, the pH-responsive polymer component is structured to discourage release and solubilization of the payload when exposed to an environment that includes a pH of about 6.0 or higher, and when exposed to an aqueous medium (e.g., RT water, boiling water).

In some embodiments, the particle preparation is stable in water for up to 6 months.

In some embodiments, the particle preparation is chemically stable in a sealed storage environment for up to 6 months at −20 C, 4 C, 25 C, 30 C and 75% RH, and/or at 40 C and 75% RH.

In some embodiments, the particle preparation maintains water activity of less than 0.3, less than 0.2, and/or less than 0.1 in a sealed storage environment for up to 6 months at −20 C, 4 C, 25 C, 30 C and 75% RH, and/or at 40 C and 75% RH.

In some embodiments, the particle preparation is chemically stable in an unsealed storage environment for up to 6 months at 25 C and 75% RH.

In some embodiments, the particle preparation is stable in direct light exposure for up to 72 hours at 37 C.

In some embodiments, the particle preparation is stable in boiling water for up to 2 hours.

In another aspect, the present embodiments are directed to a capsule comprising the particle preparation as described herein, and at least one other nutrient.

In some embodiments, the particle preparation is stable within the capsule in a sealed container at temperatures in range from about 1 C to about 30 C.

In some embodiments, the particle preparation comprises water activity less than 0.3, less than 0.2, and/or less than 0.1 in a sealed or unsealed environment at temperatures in range from about 1 C to about 30 C, and the at least one other nutrient comprises at least one probiotic.

In another aspect, the present embodiments are directed to a method of manufacturing a treated particle preparation comprising: mixing a payload component comprising a nutraceutical and a polymer component comprising a pH-responsive polymer, thereby forming a mixture; extruding the mixture to form a fiber structure; milling the fiber structure to form particles; drying the particles at a temperature in a range from about 45° C. to about 65° C., thereby forming dried particles; and treating the dried particles with nitrogen, thereby forming the treated particle preparation.

In some embodiments, the method includes packaging the treated particle preparation.

In some embodiments, milling comprises concurrent adding excipient to, and mixing excipient with, the fiber structure during milling.

In some embodiments, the present disclosure provides nutraceutical compositions that may be or comprise particles (e.g., polymer microparticles). In some embodiments, particles (e.g., polymer microparticles) may comprise a polymer component (e.g., pH-responsive polymer component). Other pH-responsive polymer systems for nutrients have been described in U.S. Pat. No. 9,649,279B2. Nutraceutical compositions (e.g., particle preparations) described herein improves upon these previous descriptions.

In some instances, a pH-responsive polymer component (e.g., for use in environments with pH less than about 5) may be or comprises a copolymer comprising methacrylate (e.g., butyl methacrylate, 2-dimethylaminoethyl methacrylate, methyl methacrylate, and poly(butylmethacrylate-co-(2-dimethylaminoethyl) methacrylate-co-methylmethacrylate)) a polygalactomannan (e.g., guar gum), celluloses (ethyl cellulose, methyl cellulose, hydroxypropyl methylcellulose), a polysaccharide (e.g., chitosan), or a combination thereof.

In some instances, a pH-responsive polymer component (e.g., for use in environments with pH greater than about 6 comprises a copolymer comprising methacrylates (e.g., poly(methacrylic acid-co-ethyl acrylate) 1:1, methyl methacrylate, ethyl methacrylate, poly(methacrylic acid-co-methyl methacrylate) 1:1, poly(methyl acrylate-co-methyl methacrylate-co-methacrylic acid) 7:3:1), celluloses (ethyl cellulose, methyl cellulose, hydroxypropyl methylcellulose), a polysaccharide (e.g., hyaluronic acid), or a combination thereof.

In some cases, a biocompatible polymer component can facilitate processing of polymers and payloads, since biocompatible polymer components are desirable for nutraceutical particle preparations, foods, and beverages. A biocompatible component may be or comprises hyaluronic acid, beta-cyclodextrin, cyclodextrin, chitosan, inulin, alginate, gelatin, maltodextrin, hydroxypropyl methyl cellulose, cellulose, sodium carboxyl methyl cellulose, polyethylene glycol, poly(butylene oxide), polycaprolactone, poly(ethylene oxide), poly(lactic acid), poly(lactic-co-glycolic acid), poly(vinyl alcohol), and poly(vinyl acetate).

In some cases, a pH-responsive polymer component is also temperature-responsive. A temperature-responsive polymer may be or comprises a copolymer comprising methacrylate (e.g., butyl methacrylate, 2-dimethylaminoethyl methacrylate, methyl methacrylate, and poly(butylmethacrylate-co-(2-dimethylaminoethyl) methacrylate-co-methylmethacrylate)) a polygalactomannan (e.g., guar gum), celluloses (ethyl cellulose, methyl cellulose, hydroxypropyl methylcellulose), a polysaccharide (e.g., chitosan), or a combination thereof.

In some cases, a temperature-responsive polymer component can facilitate processing of polymers and payloads, since temperature manipulation of polymer and payloads mixtures enables mixing of components and facilitates transitions of materials from flowable homogenous liquid states to solid particulate states.

In some cases, a temperature-responsive polymer is more readily processed at lower temperatures (e.g., glass transition temperature) through addition of payloads or plasticizers. In some embodiments, payloads alone can lower the glass transition temperature of temperature-responsive polymers. Collectively, this facilitates manufacturing and processing approaches at lower temperatures, since polymer component and payload component can more easily transition from flowable homogenous liquid states to solid states (e.g., particles).

In some embodiments, the present disclosure provides nutraceutical compositions that may be or comprise particles (e.g., polymer microparticles) comprising a payload component (e.g., a nutraceutical payload component). In some embodiments a payload component is or comprises a nutraceutical. In some embodiments, a nutraceutical is or comprises at least one micronutrient, macronutrient, mineral, antioxidant, carotenoid compound, protein, amino acid, fish oil, probiotic, prebiotic, vitamin, or combinations thereof. In some instances, a nutraceutical is fat soluble. In some instances, a nutraceutical is water soluble. In some instances, a nutraceutical is soluble in organic solvents. In some instances, a nutraceutical is soluble in gastrointestinal fluids. In some embodiments, a payload component is or comprises at least one or carotenoid compound. In some embodiments, a carotenoid compound comprises lutein, zeaxanthin, or a combination thereof.

In some instances, a provided nutraceutical composition (e.g., that is or comprises a particle preparation) provides for release of payload component (e.g., a nutraceutical payload component).

In some instances, release of payload component may occur at least in part due to at least partial dissolution (e.g., in a liquid medium) of polymer component. In some instances, greater than about 10%, about 20%, about 30%, about 40%, about 50%, about 60%, about 70%, about 80%, about 90%, about 95%, about 99% release of payload component may occur at least in part due to at least partial dissolution (e.g., in a liquid medium) of polymer component.

In some instances, release of payload component may occur when particle preparations (e.g., nutraceutical compositions) are in an environment with a particular pH range. In some instances, release of payload component occurs in an environment with a low pH (e.g., wherein pH is less than about 5, e.g., in the stomach where pH is less than about 5). In some instances, release of payload component may occur when particle preparations (e.g., nutraceutical compositions) are in an environment with a pH in the range of about 1 to about 5.

In some embodiments, release of payload component when exposed to a pH and/or temperature similar to that of a gastrointestinal tract (e.g., a pH in the range of about 1 to about 5, a temperature in the range of about 30° C. to about 43° C.).

In some instances, release occurs in a neutral and/or high pH environment (e.g., wherein pH is greater than about 6, in the intestines where pH is greater than about 6). In some instances, release of payload component may occur when particle preparations (e.g., nutraceutical compositions) are in an environment with a pH in the range of about 5.5 to about 8.

In some instances, the pH-responsive polymer provides release of the nutrient upon exposure to a pH and/or temperature in the gastrointestinal tract (e.g., a pH in the range of about 5.5 to about 8.0, a temperature in the range of about 30° C. to about 43° C.). In some such embodiments, no significant release occurs (or significantly less release occurs) at higher pH; alternatively or additionally, in some such embodiments, no significant release occurs (or significantly less release occurs) at lower pH.

In some instances, release of at least about 80%, at least about 85%, at least about 90%, or at least about 95% of payload component (e.g., a nutraceutical component) in a particle preparation (i.e., nutraceutical composition) may occur within about 5 minutes, about 10 minutes, about 15 minutes of being in an environment with a particular pH range.

In some instances, release of at least about 80% of payload component (e.g., a nutraceutical component) in a particle preparation (i.e., nutraceutical composition) may occur within about 5 minutes of being in an environment with a pH less than about 5.

In some instances, release of at least about 90% of payload component (e.g., a nutraceutical component) in a particle preparation (i.e., nutraceutical composition) may occur within about 15 minutes of being in an environment with a pH less than about 5.

In some instances, release of at least about 95% of payload component (e.g., a nutraceutical component) in a particle preparation (i.e., nutraceutical composition) may occur within about 15 minutes of being in an environment with a pH less than about 5.

In some instances, release of at least about 90% of payload component (e.g., a nutraceutical component) in a particle preparation (i.e., nutraceutical composition) may occur within about 15 minutes of being in an environment with a pH less than about 5.

In some instances, release of at least about 95% of payload component (e.g., a nutraceutical component) in a particle preparation (i.e., nutraceutical composition) may occur within about 15 minutes of being in an environment with a pH less than about 5.

In some instances, release of at least about 90% of payload component (e.g., a nutraceutical component) in a particle preparation (i.e., nutraceutical composition) may occur within about 15 minutes of being in an environment with a pH of about 5.5 to about 8.

In some instances, release of at least about 95% of payload component (e.g., a nutraceutical component) in a particle preparation (i.e., nutraceutical composition) may occur within about 15 minutes of being in an environment with a pH of about 5.5 to about 8.

In some instances, a provided particle preparation (i.e., nutraceutical composition) (e.g., that is or comprises a particle preparation) may be or are effective at protecting payload component (e.g., nutraceutical payload component) against a physical change, a chemical change, or both (e.g., degradation, oxidation, hydrolysis, isomerization, fragmentation, or a combination thereof). In some instances a physical or chemical change may be induced by one or more of heat, light, or water. In some instances, degradation of a payload component (e.g., a nutraceutical payload component) is characterized by utilizing HPLC to compare physical characteristics of a payload component after being incorporated in particle preparations to physical characteristics before being incorporated in particle preparations.

In some instances, a payload component (e.g., nutraceutical payload component) may be or is protected against oxidation. In some instances, at least 80% of the payload component remains chemically stable for at least two months at ambient temperature.

In some instances, a provided particle preparation (i.e., nutraceutical composition) (e.g., that is or comprises a particle preparation) may be or remain stable, e.g., to store for a particular period of time under particular conditions. For example, in some embodiments, 99% of a payload component present in a provided composition at a particular point in time remains present, and/or one or more size characteristics (e.g., average diameter and/or one or more features of size distribution of a particle composition) remains stable throughout a period of time during which the composition is maintained under particular conditions. In some embodiments, the period of time is at least 2, 3, 4, 5, 6, 7,8, 9, 10, 11, 12, 13, 14, 15, 16, 17, 18, 19, 20, 21, 22, 23, 24 weeks or more, and/or at least about 1, 2, 3, 4, 5, 6, 7, 8, 9, 10, 11, 12 months or more, and/or at least about 1, 2, 3, 4, 5 years or more. In some such embodiments, the particular conditions comprise ambient temperature; in some such embodiments, the particular conditions comprise elevated (above ambient) temperature. Alternatively or additionally, in some embodiments, the particular conditions comprise aqueous conditions (e.g., aqueous liquid conditions). In some embodiments, the period of time is at least two months and the particular conditions comprise ambient temperature.

In some instances, disclosed particle preparations (e.g., nutraceutical compositions) may be or are effective to protect against permeation of water. As used herein, protection against permeation of water should be not be construed to be limited as to occurring only in water. In some instances, protection against permeation of water may occur in an environment in which water may be present and/or introduced. For example, protection against permeation of water may occur in water, aqueous-based liquid, consumable liquid (e.g., milk, juice, etc.) non-aqueous-based liquid, oils, and/or dry environments.

In some instances, disclosed particle preparations (e.g., nutraceutical compositions) may be or are effective to protect against permeation of water. In some instances, the formulation is stable (>70% chemical stability) up to 8 weeks in water.

In some instances, disclosed particle preparations (e.g., nutraceutical compositions) may be or are effective to protect against permeation of water, for example, in water, aqueous-based liquid, consumable liquid (e.g., milk, juice, etc.), non-aqueous-based liquid, oils, or dry environments. In some instances, the formulation is stable (>65% chemical stability) up to about 200 days, about 1 year, about 2 years, about 3 years, up to 4 years, about 5 years in water, aqueous-based liquid, consumable liquid (e.g., milk, juice, etc.), non-aqueous-based liquid, oils, or dry environments. This is the first reported instance of liquid stability for lutein and zeaxanthin, both in general and in particle preparations.

In some instances, less than 10% of payload component (e.g., nutraceutical component) is released after soaking a provided particle preparation (i.e., nutraceutical composition) (e.g., that is or comprises a particle preparation) in water for 2 hours at 100° C. In some instances, less than 10% of payload component) is released from particles after soaking a provided particle preparation (i.e., nutraceutical composition) in water for 2 hours at 25° C.

In some instances, disclosed particle preparations (e.g., nutraceutical compositions) are stable (>50% chemical stability) up to 2 weeks in milk.

In some instances, particle preparations (e.g., nutraceutical compositions) are stable (>99.99% probiotic viability) when combined with a probiotic powder. In some instances, the formulation does not induce viability loss of probiotics when combined with probiotic powder. This is the first reported instance of maintaining probiotic stability when combined with lutein and zeaxanthin, both in general and in particle preparations.

In some cases, when a particle preparation (i.e., nutraceutical composition) is placed in low pH solution (e.g., simulated gastric fluid, pH 1.2), >95% release of payload component may be observed within 15 mins, while <5% release may be observed for formulations incubated at ambient temperature or boiling water over a 2 hour period.

In some instances, particle preparations (e.g., nutraceutical compositions) may be effective to protect payload component against light-induced degradation. In some instances, payload component is stable (>65% chemical stability) when exposed to light (>80,000 lux) at elevated temperatures (37° C.) for up to 72 hours.

In some instances, release of payload component at desired pH (either <5 or between 5.5 and 8) is maintained after storage in a freezer (−85° C. to 0° C.), a refrigerator (1-10° C.), or atmospheric temperature (−10° C.-40° C.) for time periods between 0-1 week, 0-1 month, 0-1 years, or 1-5 years of storage.

In some instances, protection against heat, light, water, and oxidation of payload component is maintained after storage in a freezer (−85° C. to 0° C.), a refrigerator (1-10° C.), or atmospheric temperature (−10° C.-40° C.) for time periods between 0-1 week, 0-1 month, 0-1 year, or 1-5 years of storage.

In some instances, low residual solvent content of nutraceutical compositions (e.g., particle preparations) is maintained after storage in a freezer (−85° C. to 0° C.), a refrigerator (1-10° C.), or atmospheric temperature (−10° C.-40° C.) for time periods between 0-1 week, 0-1 month, 0-1 year, or 1-5 years.

In some instances, low water activity of nutraceutical compositions (e.g., particle preparations comprising a nutraceutical payload) is maintained (less than 0.2000 water activity) after storage in a freezer (−85° C. to 0° C.), a refrigerator (1-10° C.), or atmospheric temperatures (−10° C.-40° C.) for time periods between 0-1 week, 0-1 month, 0-1 year or 1-5 years.

In some instances, nutraceutical compositions (e.g., particle preparations comprising a nutraceutical payload component) exhibit improved anti-caking, anti-clumping, anti-agglomerating, and/or anti-aggregating performance over a polymer component with or without the nutraceutical composition when exposed to and maintained at ambient (22° C.) or elevated (35° C. or 50° C.) temperatures for time periods between 0-1 day, 0-1 week, 0-1 month, 0-1 year or 1-5 years.

In some instances, nutraceutical compositions (e.g., particle preparations comprising a nutraceutical payload) exhibit improved anti-caking, anti-clumping, anti-agglomerating and/or anti-aggregating performance over the particle preparation in the presence of excipients (e.g., microcrystalline cellulose, starches, etc.) when exposed maintained to ambient (22° C.) or elevated (35° C. or 50° C.) temperatures for time periods between 0-1 day, 0-1 week, 0-1 month, 0-1 year or 1-5 years.

In some embodiments, a particle preparation may further comprise an excipient component (e.g., an anti-caking component, an anti-clumping component, an anti-agglomerating component, and/or an anti-aggregating component [e.g., any of an excipient comprising microcrystalline cellulose, starches, etc.], wherein an excipient component is at least about 99 wt %, at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., a nutraceutical composition).

We further provide insight that disclosed nutraceutical compositions (e.g., particle preparations comprising a nutraceutical payload) are particularly useful for stabilizing payload components in consumable compositions (e.g., a food product, a beverage product, an animal-consumable product, dry powders, etc.), combining with probiotic components that lose viability when exposed to high water activity entities (e.g., existing formulations for carotenoids such as lutein and/or zeaxanthin), or both.

No known products combine particle preparations (e.g., particle preparations [e.g., nutraceutical compositions] comprising carotenoids, e.g., lutein and zeaxanthin) with probiotic components because technologies have not previously been developed to enable combination of nutraceutical compositions (e.g., particle preparations [e.g., nutraceutical compositions] comprising carotenoids, e.g., lutein and zeaxanthin) in a way that preserves probiotic viability (e.g., colony forming units) in the presence of high water activity components.

In certain embodiments, the present disclosure provides consumable compositions (e.g., a food product, a beverage product, an animal-consumable product, dry powders, etc.) comprising disclosed nutraceutical compositions, at least one probiotic, or a combination thereof. In some instances, particle preparations (e.g., nutraceutical compositions) further comprise at least one probiotic.

In some cases, particle preparations (e.g., nutraceutical compositions) comprising low residual solvent content, having low water activity, or both may be used to stabilize payload components in consumable compositions (e.g., a food product, a beverage product, an animal-consumable item, dry powders, etc.). In some aspects, provided particle preparations (e.g., nutraceutical compositions) are or may be useful for improving health or longevity in animals. In some aspects, provided consumable compositions are or may be useful for improving health or longevity in animals.

In some aspects, the present disclosure provides methods of promoting health or longevity in animals, for example providing an effective amount of particle preparations (e.g., nutraceutical compositions) described herein to an animal. In some cases, the particle preparation (i.e., nutraceutical composition) is provided in a consumable composition (e.g., a food product, a beverage product, an animal consumable-item).

In some cases, animals may be human. In some instances, a human may be for example, an adult, an elder, a teenager, an adolescent, an infant, or a prenatal human.

In some cases, animals may be an agricultural animal, for example, a cow, a horse, a pig, a sheep, a goat, a domesticated bird (e.g., chicken, duck, goose), a non-domesticated (e.g., wild) bird, etc.

In some cases, animals may be a pet animal, for example, a dog, a cat, a rabbit, or a fish.

In some aspects, consumable compositions comprising particle preparations (e.g., nutraceutical compositions) may be edible. In some aspects, an edible composition may be a protein bar, a cereal, a protein powder, a salad dressing, a nutritional supplement, a baby formula, a smoothie, a yoghurt, an ice cream, a sachet, a spice, a food additive, a candy, a sprinkle packet, a pet food, an agricultural seed, a dry powder, or a fertilizer.

In some aspects, consumable compositions comprising particle preparations (e.g., nutraceutical compositions) are drinkable. In some aspects, a drinkable composition may be a sports drink, beer, wine, tea, coffee, milk, juice, water, or a liquid pharmaceutical formulation.

In some instances, the present disclosure provides for preparations of formulations comprising pH-responsive polymers associated with (e.g., encapsulating and/or otherwise complexed with) one or more nutrients or payloads, thereby providing compositions and methods for storage in food and/or beverage products.

In some instances, release of payload component at desired pH (e.g., either <5 or between 5.5 and 8) may be maintained after storage (e.g., with or within a consumable composition) in a freezer (−85° C. to 0° C.), a refrigerator (1-10° C.), or atmospheric temperature (−10° C.-40° C.). In some instances, for time periods between 0-1 week, 0-1 month, 0-1 years, or 1-5 years of storage.

In some instances, protection against heat, light, water, and oxidation of payload component is maintained after storage (e.g., with or within a consumable composition) in a freezer (−85° C. to 0° C.), a refrigerator (1-10° C.), or atmospheric temperature (−10° C.-40° C.) for time periods between 0-1 week, 0-1 month, 0-1 year or 1-5 years.

In some instances, low residual solvent content of particle preparations (e.g., nutraceutical compositions) is maintained after storage (e.g., with or within a consumable composition) in a freezer (−85° C. to 0° C.), a refrigerator (1-10° C.), or atmospheric temperature (-10° C.-40° C.) for time periods between 0-1 week, 0-1 month, 0-1 year or 1-5 years.

In some instances, low water activity (e.g., water activity less than about 0.2) of particle preparations (e.g., nutraceutical compositions) is maintained after storage (e.g., with or within consumable compositions) in a freezer (−85° C. to 0° C.), a refrigerator (1-10° C.), or atmospheric temperature (−10° C.-40° C.) for time periods between 0-1 week, 0-1 month, 0-1 year or 1-5 years.

In some instances, anti-caking, anti-clumping, anti-agglomerating, and/or anti-aggregating of particle preparations (e.g., nutraceutical compositions) is maintained after storage (e.g., with or within consumable compositions) in a freezer (−85° C. to 0° C.), a refrigerator (1-10° C.), or atmospheric temperature (−10° C.-50° C.) for time periods between 0-1 week, 0-1 month, 0-1 year or 1-5 years.

In some instances, anti-caking or anti-clumping or anti-agglomerating or anti-aggregating of particle preparations (e.g., nutraceutical compositions) is maintained when mixed with excipients (e.g., microcrystalline cellulose, starches, etc.) after storage (e.g., with or within consumable compositions) in a freezer (−85° C. to 0° C.), a refrigerator (1-10° C.), or atmospheric temperature (−10° C.-50° C.) for time periods between 0-1 week, 0-1 month, 0-1 year or 1-5 years.

In another aspect, the present embodiments are directed to a method of compound extraction and quantification, the method comprising: extracting at least one of lutein and zeaxanthin from at least one microparticle.

In some embodiments, lutein and zeaxanthin are co-encapsulated together within the at least one microparticle, and extracting comprises acetone-based extraction.

In some embodiments, the at least one microparticle is incorporated into at least one of milk powder, liquid milk, and a capsule.

In some embodiments, the at least one microparticle comprises lutein or zeaxanthin, and extracting comprises DCM-based extraction.

In some embodiments, the at least one microparticle is incorporated into a capsule, and the capsule comprises from about 10 to about 15 different nutraceuticals.

In another aspect, the present embodiments are directed to a quantification method for encapsulated lutein and zeaxanthin levels in formulation by HPLC, according to Appendix A.

In another aspect, the present embodiments are directed to a quantification method for lutein and zeaxanthin levels from powder milk solution by HPLC, according to Appendix B.

In another aspect, the present embodiments are directed to a quantification method for lutein and zeaxanthin levels from milk by HPLC, according to Appendix C.

In another aspect, the present embodiments are directed to a quantification method for lutein and zeaxanthin levels from probiotic capsules by HPLC, according to Appendix D.

In another aspect, the present embodiments are directed to a quantification method for encapsulated lutein levels in formulations by HPLC, according to Appendix E.

In another aspect, the present embodiments are directed to a quantification method for lutein levels from powder milk solution by HPLC, according to Appendix F.

In another aspect, the present embodiments are directed to a quantification method for lutein levels from milk by HPLC, according to Appendix G.

In another aspect, the present embodiments are directed to a quantification method for lutein levels from probiotic capsules by HPLC, according to Appendix H.

In another aspect, the present embodiments are directed to a quantification method for encapsulated zeaxanthin levels in formulation by HPLC, according to Appendix I.

In another aspect, the present embodiments are directed to a quantification method for zeaxanthin levels from powder milk solution by HPLC, according to Appendix J.

In another aspect, the present embodiments are directed to a quantification method for zeaxanthin levels from milk by HPLC, according to Appendix K.

In another aspect, the present embodiments are directed to a quantification method for zeaxanthin levels from probiotic capsules by HPLC, according to Appendix L.

In some embodiments, the present disclosure provides compositions that are or comprise a particle preparation, wherein the particles comprise (i) a polymer component; and (ii) a payload component, wherein the polymer component comprises a pH-responsive polymer; and the payload component comprises a nutraceutical (e.g., a carotenoid compound such as lutein and/or zeaxanthin, or a vitamin such as vitamin D), and wherein the particle preparation has low solvent content and/or low water activity. In some such embodiments, the composition and/or the particle preparation is characterized in that the payload component shows increased stability (e.g., is protected against one or more of degradation, oxidation, other physical and/or chemical changes) when exposed to one or more environmental conditions such as, for example, light, elevated temperature, presence of water, and/or in the context of a complex material. Alternatively or additionally, in some such embodiments, the compositions and/or the particle preparation is characterized in that it releases the payload component in environments with a certain pH (e.g., low pH such as is found at certain gastric locations), or within a certain range of pH, and not in environments with other pH. In some embodiments, a provided composition (e.g., that is or comprises such a particle preparation) has been stored for a period of time under particular conditions, e.g., as described herein, and substantially maintains one or more features of the composition as present prior to the period of time.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

Aspects and embodiments of the present embodiments are set forth with particularity in the appended claims. A better understanding of certain features and advantages of various aspects of the present disclosed embodiments may be obtained by reference to the following detailed description that sets forth illustrative embodiments, e.g., in which the principles of the embodiments are utilized, and the accompanying figures of the drawing, of which:

FIG. 1A shows, in a non-limiting example, bright field and scanning electron microscopy images of an exemplary particle preparation comprising lutein as payload component.

FIG. 1B shows, in a non-limiting example, bright field and scanning electron microscopy images of an exemplary particle preparation comprising zeaxanthin as payload component. FIG. 1C shows, in a non-limiting example, bright field and scanning electron microscopy images of an exemplary particle preparation with payload component comprising vitamin D. FIG. 1D shows, in a non-limiting example, bright field and scanning electron microscopy images of an exemplary particle preparation with payload components comprising both lutein and zeaxanthin.

FIG. 2A presents certain particle size distribution characteristics of exemplary particle preparations for lutein as described herein (e.g., particle diameter distributions). FIG. 2B presents certain particle size distribution characteristics of exemplary particle preparations for zeaxanthin as described herein (e.g., particle diameter distributions). FIG. 2C presents certain particle size distribution characteristics of exemplary particle preparations for vitamin D as described herein (e.g., particle diameter distributions). FIG. 2D presents certain particle size distribution characteristics of exemplary particle preparations for lutein and zeaxanthin as described herein (e.g., particle diameter distributions).

FIGS. 3A-C present plots of actual loading achieved with exemplary particle preparations as described herein with lutein (FIG. 3A), zeaxanthin (FIG. 3B), Vitamin D (FIG. 3C), or both lutein and zeaxanthin (FIG. 3D) payloads and demonstrates that payload concentration can be controlled, for example, by selecting or adjusting the ratio of initial payload component to initial polymer component.

FIGS. 4A-B shows, in a non-limiting example, particle preparations protect lutein (FIG. 4A), zeaxanthin (FIG. 4A), and vitamin D (FIG. 4B) from damage arising from light exposure.

FIG. 5 shows, in a non-limiting example, particle preparations protect lutein and zeaxanthin from damage arising from exposure to water.

FIG. 6 shows, in a non-limiting example, particle preparations enable storage of lutein and zeaxanthin in milk.

FIGS. 7A-C shows, in a non-limiting example, particle preparations provide controlled lutein (FIG. 7A), zeaxanthin (FIG. 7B), and vitamin D (FIG. 7C) release in acidic conditions, minimizing release in ambient temperature and boiling water.

FIG. 8 shows, in a non-limiting example, particle preparations exhibit lower water activity than competitor products.

FIG. 9 shows, in a non-limiting example, particle preparations blend homogenously with commercially available probiotics whereas competitor products exhibit heterogeneous mixing.

FIG. 10 shows, in a non-limiting example, particle preparations retain over 99.99% of Lacticaseibacillus rhamnosus probiotic viability upon mixing in the dry powdered state, as compared to control without microparticles.

FIG. 11 shows, in a non-limiting example, particle preparations retain over 99.99% of Lacticaseibacillus rhamnosus probiotic viability upon mixing in water for 3 hours at 37° C., as compared to control without microparticles.

FIG. 12 shows, in a non-limiting example, particle preparations protect vitamin D from damage arising from exposure to boiling water and high temperatures.

FIGS. 13A-B shows, in a non-limiting example, particle preparations with nutraceutical payloads mitigate caking, agglomeration, aggregation, or clumping whereas polymer component alone (without payload) exhibits clumping at 50° C.

FIGS. 14A-C shows, in a non-limiting example, particle preparations with nutraceutical payloads and added excipients mitigate caking, agglomeration, aggregation, or clumping at temperatures 25° C. (FIG. 14A), 35° C. (FIG. 14B), or 50° C. (FIG. 14C) whereas particle preparations with nutraceutical payloads without excipients cannot.

FIG. 15 shows, in a non-limiting example, particle preparations with nutraceutical payloads have reduced glass transition temperatures, as compared to payloads alone or polymer component alone.

FIG. 16 shows, in a non-limiting example, (A) improved retention of chemical stability (measured via HPLC) for zeaxanthin microparticles (black line) as compared to commercial product OmniActive Lutemax 2020 at 4 C, retention of chemical stability (measured via HPLC) of (B) lutein and (C) zeaxanthin in 20% lutein and 3.6% zeaxanthin microparticles at 0 and 16 weeks at 4 C.

FIG. 17 shows, in a non-limiting example, several exemplary release profiles of various microparticle formulations: (A) VitaKey microparticle lutein, (B) OmniActive Lutemax 2020, (C) VitaKey microparticle Vitamin D, and (D) BASF microparticle Vitamin D in various conditions (room temperature water, 100 C boiling water, and 37 C simulated gastric fluid).

FIG. 18 shows, in a non-limiting example, a schematic of an extrusion method used to manufacture lutein and zeaxanthin, or vitamin D, microparticles of low solvent and low water activity.

FIG. 19 shows, in a non-limiting example, maintenance of chemical stability (measured via HPLC) of (A) lutein and (B) zeaxanthin during manufacturing processes of (1) extrusion, (2) extrusion and milling, and (3) extrusion, milling, then baking.

FIG. 20 shows, in a non-limiting example, maintenance of chemical stability (measured via HPLC) of (A) lutein and (B) zeaxanthin for 10% lutein/1.8% zeaxanthin (black bars) and 20% lutein/3.6% zeaxanthin (grey bars) during baking at 55 C for up to 24 hours.

FIG. 21 shows, in a non-limiting example, maintenance of chemical stability (measured via HPLC) of (A) lutein and (B) zeaxanthin for 10% lutein/1.8% zeaxanthin (black bars) and 20% lutein/3.6% zeaxanthin (grey bars) during exposure to high humidity at room temperature for up to 24 hours.

FIG. 22 shows, in a non-limiting example, maintenance of chemical stability (measured via HPLC) of (A) lutein and (B) zeaxanthin for 10% lutein/1.8% zeaxanthin (black bars) and 20% lutein/3.6% zeaxanthin (grey bars) during exposure to oxygen (˜21% in air) at room temperature for up to 24 hours.

FIG. 23 shows, in a non-limiting example, maintenance of chemical stability (measured via HPLC) of lutein at (A) 25 C and (B) 4 C free lutein, for 10% lutein/1.8% zeaxanthin, 20% lutein/3.6% zeaxanthin, and OmniActive Lutemax 2020, during incubation in water for up to 6 months.

FIG. 24 shows, in a non-limiting example, maintenance of chemical stability (measured via HPLC) of zeaxanthin at (A) 25 C and (B) 4 C free zeaxanthin, for 10% lutein/1.8% zeaxanthin, 20% lutein/3.6% zeaxanthin, and OmniActive Lutemax 2020, during incubation in water for up to 6 months.

FIG. 25 shows, in a non-limiting example, maintenance of chemical stability (measured via HPLC) of lutein in (A) 10% lutein/1.8% zeaxanthin or (B) 20% lutein/3.6% zeaxanthin microparticles stored at −20 C, 4 C, 25 C, or 30 C at 75% relative humidity for up to 6 months.

FIG. 26 shows, in a non-limiting example, maintenance of chemical stability (measured via HPLC) of lutein in (A) 10% lutein/1.8% zeaxanthin or (B) 20% lutein/3.6% zeaxanthin microparticles stored at 40 C at 75% relative humidity for up to 6.6 months.

FIG. 27 shows, in a non-limiting example, maintenance of chemical stability (measured via HPLC) of zeaxanthin in (A) 10% lutein/1.8% zeaxanthin or (B) 20% lutein/3.6% zeaxanthin microparticles stored at −20 C, 4 C, 25 C, or 30 C at 75% relative humidity for up to 6 months.

FIG. 28 shows, in a non-limiting example, maintenance of chemical stability (measured via HPLC) of zeaxanthin in (A) 10% lutein/1.8% zeaxanthin or (B) 20% lutein/3.6% zeaxanthin microparticles stored at 40 C at 75% relative humidity for up to 6.6 months.

FIG. 29 shows, in a non-limiting example, maintenance of low water activity (A) 20% lutein/3.6% zeaxanthin or (B) 10% lutein/1.8% zeaxanthin microparticles stored at −20 C, 4 C, 25 C, 30 C at 75% relative humidity, or 40 C at 75% relative humidity for up to 6 months.

FIG. 30 shows, in a non-limiting example, maintenance of chemical stability (measured via HPLC) in (A) dry conditions (<10% relative humidity) or (B) humid conditions (75% relative humidity) of free vitamin D, Vitamin D microparticles at 1% loading, Vitamin D microparticles at 2% loading, and Vitamin D microparticles at 5% loading for up to 12 weeks.

FIG. 31 shows, in a non-limiting example, maintenance of chemical stability (measured via HPLC) of free vitamin D, Vitamin D microparticles at 10% loading, Vitamin D microparticles at 2% loading, BASF vitamin D microparticles, and DSM Vitamin D microparticles at 85000 lux exposure for 72 hours at 37 C.

FIG. 32 shows, in a non-limiting example, maintenance of chemical stability (measured via HPLC) of free vitamin D, Vitamin D microparticles at 10% loading, Vitamin D microparticles at 1% loading (with 0.25% vitamin E), Vitamin D microparticles at 1% loading, Vitamin D microparticles at 2% loading, Vitamin D microparticles at 5% loading, and DSM Vitamin D microparticles at after 2 hours in boiling water at 100 C.

FIG. 33 shows, in a non-limiting example, maintenance of chemical stability (measured via HPLC) of lutein formulated in 20% lutein/3.6% zeaxanthin microparticles, stored at 25 C (black bars) and 4 C (grey bars) in (A) unopened and (B) opened conditions.

FIG. 34 shows, in a non-limiting example, maintenance of chemical stability (measured via HPLC) of zeaxanthin formulated in 20% lutein/3.6% zeaxanthin microparticles, stored at 25 C (black bars) and 4 C (grey bars) in (A) unopened and (B) opened conditions.

FIG. 35 shows, in a non-limiting example, maintenance of probiotic viability (colony forming units; measured via agar plating) of HN001 Lacticaseibacillus rhamnosus contained within the same capsule with 20% lutein/3.6% zeaxanthin microparticles, stored at 25 C (black bars) and 4C (grey bars) in (A) unopened and (B) opened conditions.

FIG. 36 shows, in a non-limiting example, maintenance of probiotic viability (colony forming units; measured via agar plating) of HN019 Bifidobacterium animalis subsp. lactis contained within the same capsule with 20% lutein/3.6% zeaxanthin microparticles, stored at 25 C (black bars) and 4 C (grey bars) in (A) unopened and (B) opened conditions.

FIG. 37 shows, in a non-limiting example, a schematic of a mixing and milling method used to manufacture lutein and zeaxanthin, or vitamin D, microparticles of low solvent and low water activity that mix well into aqueous and/or non-aqueous liquids.

FIG. 38 shows, in a non-limiting example, a loading and composition details for various formulations of increased water dispersibility.

FIG. 39 shows, in a non-limiting example, exemplary images of the formulations (F0-F11) listed in FIG. 38.

FIG. 40 shows, in a non-limiting example, exemplary images of the formulations (F12-F21, OmniActive, and L+Z) listed in FIG. 38.

FIG. 41 shows, in a non-limiting example, images of the formulations (F0-F2) listed in FIG. 38 dispersed in water and coffee.

FIG. 42 shows, in a non-limiting example, images of the formulations (F1, F3) listed in FIG. 38 dispersed in water and coffee at the time of addition and after 15 minutes of settling.

FIG. 43 shows, in a non-limiting example, images of the formulations (F1, F3) listed in FIG. 38 dispersed in water and coffee at the time of addition.

FIG. 44 shows, in a non-limiting example, images of the formulations listed in FIG. 38 before dispersion in liquids.

FIG. 45 shows, in a non-limiting example, images of the formulations listed in FIG. 38 (A) dispersed in water and coffee at the time of addition and images of the particles, and images of the corresponding microparticles (B) before addition to liquids, (C) after addition to water, and (D) after addition to coffee.

FIG. 46 shows, in a non-limiting example, images of the formulations listed in FIG. 38 dispersed in Body Armor beverage.

FIG. 47 shows, in a non-limiting example, images of the formulations listed in FIG. 38 dispersed in Body Armor beverage.

FIG. 48 shows, in a non-limiting example, images of the formulations listed in FIG. 38 dispersed in a commercial beverage with the commercial bottle.

FIG. 49 shows, in a non-limiting example, maintenance of chemical stability (measured via HPLC) of lutein and zeaxanthin microparticles (F2 in FIG. 38) stored in water (A) 25 C and (B) 4 C temperatures for up to 3 months.

FIG. 50 shows, in a non-limiting example, incorporation of 20% lutein and 3.6% zeaxanthin microparticles in a pectin gummy.

FIG. 51A shows, in a non-limiting example, a chromatogram of lutein.

FIG. 51B shows, in a non-limiting example, a chromatogram of zeaxanthin.

FIGS. 52A-52F show, in a non-limiting example, data plots of exemplary extraction and recovery.

FIGS. 53A-53F show, in a non-limiting example, data plots of exemplary extraction and recovery.

FIGS. 54A-54F show, in a non-limiting example, data plots of exemplary extraction and recovery.

FIG. 55 shows, in a non-limiting example, a chromatogram of lutein.

FIG. 56 shows, in a non-limiting example, a chromatogram of zeaxanthin.

### DETAILED DESCRIPTION

### Certain Terminology

Section headings used herein are for organizational purposes only and are not to be construed as limiting the subject matter described.

Unless defined otherwise, technical and scientific terms used herein have the same meaning as is commonly understood to which the claimed subject matter belongs. In the event that there are a plurality of definitions for terms herein, those in this section prevail.

It is to be understood that the general description and the detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed embodiments.

As used herein, the symbol “<” means less than or fewer than. As used herein, the symbol “>” means more than.

As used herein, the term “about” or “approximately” means within 10%, preferably within 10%, and more preferably within 5% of a given value or range.

As used herein, ambient temperature is a colloquial expression for the typical or preferred indoor (climate-controlled) temperature to which people are generally accustomed. It represents the small range of temperatures at which the air feels neither hot nor cold, approximately 21° C. In some embodiments, ambient temperature is 25±5° C. In some embodiments, ambient temperature is 18° C. In some embodiments, ambient temperature is 19° C. In some embodiments, ambient temperature is 20° C. In some embodiments, ambient temperature is 21° C. In some embodiments, ambient temperature is 22° C. In some embodiments, ambient temperature is 23° C. In some embodiments, ambient temperature is 24° C. In some embodiments, ambient temperature is 25° C. In some embodiments, ambient temperature is 26° C. In some embodiments, ambient temperature is 27° C. In some embodiments, ambient temperature is 28° C. In some embodiments, ambient temperature is 29° C. In some embodiments, ambient temperature is 30° C.

Definition of standard chemistry terms may be found in reference works, including but not limited to, Carey and Sundberg “Advanced Organic Chemistry 4th Ed.” Vols. A (2000) and B (2001), Plenum Press, New York.

As used herein, the term “nutraceutical composition” refers to a substance or material that is or comprises a nutraceutical agent (e.g., a nutraceutical). Those skilled in the art will be aware of a variety of agents understood in the art to be nutraceutical agents such as, for example, agents that are or comprise one or more antioxidants, macronutrients, micronutrients, minerals, prebiotics, probiotics, prebiotics, vitamins, or combinations thereof. In some embodiments, a nutraceutical is or comprises a carotenoid compound such as alpha-lipoic acid, astaxanthin, adonixanthin, adonirubin, beta-carotene, coenzyme Q10, lutein, lycopene, or zeaxanthin. In some embodiments, a nutraceutical is or comprises a vitamin such as vitamin D. In many embodiments, a nutraceutical agent is a natural product, and in certain such embodiments it is a product produced by plants. Many nutraceutical agents are compounds that have been reported or demonstrated to confer a benefit or provide protection against a disease in an animal or a plant. In some cases, nutraceuticals may be used to improve health, delay the aging process, protect against chronic diseases, increase life expectancy, or support the structure or function of the body of an animal, such as a human, a pet animal, an agricultural animal, or another domesticated animal. In some embodiments, a provided particle preparation (i.e., nutraceutical composition) is used as and/or is included in a nutritional supplement or other consumable (e.g., food) for a human or animal, a fertilizer for a plant, or animal feed.

As used herein, the term “encapsulated” is used to refer to a characteristic of being physically associated with, and in some embodiments partly or wholly covered or coated. For example, in many embodiments of the present disclosure, a payload component is described as being encapsulated by a polymer component.

As used herein, the term “degradation” refers to a change in chemical structure and typically involves breakage of at least one chemical bond. To say that a chemical compound is degraded means that that the chemical structure of the chemical compound has changed (e.g., a chemical bond is broken). Common mechanisms of degradation include, for example, oxidation, hydrolysis, isomerization, fragmentation, or a combination thereof.

The term “pH-responsive” is used to refer to polymer components as described herein, and in particular means that the relevant polymer component is characterized in that one or more aspects of its structure or arrangement is altered when exposed to a change in pH condition (e.g., to a particular pH and/or to a change of particular magnitude). In some embodiments, a polymer component is considered to be “pH-responsive” if, when the relevant polymer component is associated with a payload component in a particle preparation as described herein, the particle preparation releases the payload component under specific pH condition(s). In some embodiments, >90% of payload component is released from a particle preparation that includes a pH-responsive polymer component within 15 minutes when the particle preparation is exposed to a particular defined pH condition (e.g., within a range of defined pH values and/or at a specific pH value); in some embodiments, such release results when such contacting occurs at temperatures between 33-40° C., and in aqueous-based buffers of ionic strength ranging from 0.001-0.151 M (e.g., water, simulated gastric fluid, gastric fluid, simulated intestinal fluid, intestinal fluid) with osmolality between 1-615 mOsm/kg. In some embodiments, a pH-responsive polymer component is one that degrades when exposed to a particular pH or pH change. Alternatively or additionally, in some embodiments, a pH-responsive polymer component is one that becomes soluble, or significantly (e.g., (e.g., by at least about 5%) increases its solubility when exposed to a particular pH level, or pH change. In some embodiments, a pH-responsive polymer component includes one or more moieties whose protonation state changes at the relevant pH or in response to the relevant pH change. For example, in some embodiments, a pH responsive polymer component includes one or more amine moieties that become protonated upon exposure to a relevant pH or pH chance.

As used herein, the term “temperature-responsive” is used to refer to polymer components as described herein, and in particular means that the relevant polymer component is characterized in that one or more aspects of its structure or arrangement is altered when exposed to a change in temperature condition (e.g., to a particular temperature and/or to a temperature change of particular magnitude). In some embodiments, a polymer component is considered to be “temperature-responsive” if, when the relevant polymer component is associated with a payload component in a particle preparation as described herein, amorphous regions of the polymer component experience a transition from a rigid state (e.g., glassy state) to a more fluid-like flexible state (e.g., more conducive to flow), at a temperature close to the point of transition from the solid state to rubbery state (e.g., glass transition).

As used herein, the term “particle” is used to refer to a small, discrete physical entity. A “particle” is not limited to a particular shape or form, for example, having a cross-section shape of a sphere, an oval, a triangle, a square, a hexagon, or an irregular shape. In some cases, particles can be solid particles. In some cases, particles can be liquid particles. In some cases, particles can be gel or gel-like particles. In some cases, particles may have a particle-in-particle structure wherein a layer of one material (e.g., one type of polymer component) encapsulates another material (e.g., another type of polymer component, which may itself encapsulate yet another, or rather may be or comprise a “core”—e.g., a polymer matrix core—of the particle). In some embodiments, a particle can have a size (e.g., a diameter) within a range. For example, a particle can have a size of about 1-3000 μm, about 1-2000 μm, about 1-1000 μm, about 1-500 μm, about 1-50 μm, about 1-300 μm, about 1-200 μm, about 1-100 μm, about 1-50 μm, about 1-25 μm, or about 1-10 μm.

As used herein, the term “layer” is used to describe a material disposed above or below a distinguishable material. In some embodiments, a particular sample or preparation (e.g., particle preparation) is described as “layered” if it is prepared via a process in which a first material is laid down and then a second material is applied atop or underneath the first (e.g., as by dipping or spraying, etc); in some such embodiments, physical or chemical distinctness of layers may be maintained over time whereas in some such embodiments, physical or chemical distinctness of layers may decay over time, at least at layer interface(s). Alternatively or additionally, in some embodiments, a particular sample or preparation may be described as layered, independent of its mode of preparation, so long as at a particular point in time and/or using a particular mode of assessment, distinct materials can be identified in a layered structure. In some embodiments, a “layered” particle may include one or more layers that wholly encapsulates a material below. In some embodiments, a “layered” particle may include one or more layers that does not wholly encapsulate a material below. In some embodiments, at least one layer of a layered preparation is or comprises a polymer, e.g., a pH responsive polymer. In some embodiments, each layer of a layered preparation is or comprises a polymer, e.g., a pH responsive polymer.

As used herein, the term “diameter” is used to refer the longest distance from one end of the particle to another end of a particle. Those skilled in the art will appreciate that a variety of techniques are available for use in characterizing particle diameters (i.e., particle sizes). In some instances, for example, size of particles (i.e., diameter of particles) can be measured by a Coulter Counter. In some instances, for example, size of particles (i.e., diameter of particles) can be measured by a Malvern Mastersizer. In some embodiments, a population of particles is characterized by an average size (e.g., D[3,2], D[4,3], etc.) and/or by particular characteristics of size distribution (e.g., absence of particles above or below particular sizes [e.g., Dv10, Dv20, Dv30, Dv40, Dv50, Dv60, Dv70, Dv80, Dv90, Dv99, etc.], a unimodal, bimodal, or multimodal distribution, etc.)

As used herein, the term “biocompatible” is used to describe a characteristic of not causing significant detectable harm to living tissue when placed in contact therewith e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

As used herein, the term “probiotic” is used to refer to compositions that are or include a live micro-organism (e.g., bacterium, fungus, virus, or bacteriophage) that is not harmful to certain animals (e.g., humans) so that it can safely be ingested thereby. In some embodiments, a probiotic is reported or known to provide one or more health benefits to a human or other animal when ingested, consumed, or administered to such human or other animal.

As used herein, the term “homogenous” means of substantially uniform structure or composition throughout.

As used herein, the term “beverage” is used to refer to a potable liquid (e.g., that can be ingested, swallowed, drunk, or consumed by a person or animal without material risk to the person or animal). For example, beverage can be or comprise beer, juice, milk, a sports drink, tea, water, etc. In some embodiments, a “beverage” may be or comprise a pharmaceutical formulation in liquid form.

As used herein, “water activity” of a material relates to how much free (i.e., available to bind or react) water is present in the material, and is typically determined as the ratio of the vapor pressure of water in a material (p) to the vapor pressure of pure water (p₀) at the same temperature. For example, a water activity of 0.80 means the vapor pressure is 80 percent of that of pure water. Water activity typically increases with temperature. Those skilled in the art will be familiar with three basic water activity measurement systems: Preventive Electrolytic Hygrometers (REH), Capacitance Hygrometers, and Dew Point Hygrometers (sometimes called chilled mirror).

As used herein, the term “residual solvent” means a solvent that remains in a material after manufacture or processing of the material. In some embodiments, level of residual solvent is assessed by HPLC, mass spec, NMR, FTIR, and/or gas chromatography.

As used herein, 1 ppm (“parts per million”) is equivalent to 1 milligram per liter (mg/L) or 1 milligram per kilogram (mg/kg).

Disclosed herein, among other things, are compositions and methods for manufacture, maintenance (e.g., storage) and/or use (e.g., administration or delivery) of a nutraceutical (e.g., a carotenoid comprising at least one of lutein and zeaxanthin and/or a vitamin such as vitamin D). In some cases, the nutraceutical is or comprises, for example, one or more antioxidants, macronutrients, micronutrients, minerals, prebiotics, probiotics, prebiotics, vitamins, or combinations thereof. In some embodiments, a nutraceutical is or comprises a carotenoid compound such as lutein or zeaxanthin. In some embodiments, a nutraceutical is or comprises a vitamin such as vitamin D.

In some aspects, the disclosure provides a particulate formulation (e.g., a particle preparation) of a nutraceutical for improving health. In some cases, one or more pH-responsive polymers (e.g., one or more pH-responsive polymer components) are used to encapsulate a nutraceutical (e.g., a nutraceutical payload component). In some embodiments, one or more pH-responsive polymers (e.g., one or more pH-responsive polymer components) are used to encapsulate a nutraceutical and resulting particles. In some cases, a polymer particle having low residual solvent content is used to encapsulate and stabilize a nutraceutical in foods and/or beverages. In many embodiments, low residual solvent content is achieved not by removing residual solvents from a particulate formulation (e.g., a particle preparation) after they are manufactured. Provided technologies provide benefits over existing products, among other things because ingestion of residual solvents poses health concerns and methods to remove residual solvents from formulations can be costly and/or inefficient.

### Particle Preparations

Among other things, the present disclosure provides nutraceutical compositions (e.g., particle preparations comprising a nutraceutical payload) that are or comprise particles (e.g., polymer microparticles). In some embodiments, particles comprise a matrix component and a payload component (e.g., nutraceutical payload component). In some instances, a matrix component is or comprises a biocompatible material comprising at least one of sugar, polysaccharide, carbohydrate, oil, fat, wax, protein, or a combination thereof. In some cases, one or more bacterial species are embedded in the matrix. In some cases, a matrix component is or comprises a polymer component (i.e., is a polymeric matrix). In some embodiments, a polymer component is or comprises a pH-responsive polymer component.

In some embodiments, provided particle preparations (e.g., nutraceutical compositions) are characterized by low organic solvent and/or by low water activity as described herein.

Some aspects of the present disclosure provide methods of making particles (e.g., that are or comprise polymeric matrices) described herein. In some cases, a provided such method involves using aqueous-based atomization. In some cases, solvent-based atomization is involved. In some embodiments, emulsion-based methods are involved. In some embodiments, particles are made by extrusion.

In some particular embodiments, where atomization is utilized, two or more fluid nozzles may be employed. In some particular embodiments, an acidic material (e.g., liquid) is passed through a first nozzle and a basic material (e.g., liquid) is passed through a second nozzle.

Without wishing to be bound by any particular theory, the present disclosure proposes that production technologies that achieve in situ neutralization (e.g., via passing an acidic material through a first nozzle and a basic material through a second nozzle) may provide particular advantages. In some such embodiments, in situ neutralization may achieve production of water-insoluble particles.

Thus, in some particular embodiments, the present disclosure provides technologies that achieve production of water-insoluble particles comprising a polymer component (e.g., a pH-responsive polymer component, e.g., BMC) and/or a payload (e.g., a nutraceutical payload, e.g., a carotenoid compound and/or a probiotic and/or a vitamin), and also provides preparations of such water-insoluble particles (e.g., that may be low solvent and/or low water activity preparations as described herein).

In some embodiments, the present disclosure provides particle preparations in which particles have a particular shape or form, for example, having a cross-section shape of a sphere, an oval, a triangle, a square, a hexagon, or an irregular shape. In some embodiments, a preparation includes particles of different shapes or forms. In some embodiments, most or substantially all or all particles in a preparation have a common shape.

In some embodiments, particles (e.g., polymer microparticles) in a provided particle preparation may have a distribution of diameters (e.g., Dv(10), Dv(20), Dv(30), Dv(40), Dv(50), Dv(60), Dv(70), Dv(80), Dv(90), Dv99, etc.) In some embodiments, particles (e.g., polymer microparticles) in a provided particle preparation (e.g., nutraceutical composition) may have an average diameter (e.g., D[3,2], D[4,3], etc.) Regardless of the shape of the particle, the “diameter” (i.e., size) of a particle is the longest distance from one end of the particle to another end of the particle.

In some instances, particles in a particle preparation as described and/or utilized herein may have a distribution of diameters (e.g., Dv(10), Dv(20), Dv(30), Dv(40), Dv(50), Dv(60), Dv(70), Dv(80), Dv(90), Dv(99), etc.) of up to about 3000 μm, up to about 2000 μm, up to about 1000 μm, of up to about 500 μm, up to about 400 μm, up to about 300 μm, up to about 200 μm, up to about 100 μm, up to about 50 μm, up to about 40 μm, up to about 30 μm, up to about 20 μm, up to about 10 μm, up to about 5 μm, or up to about 1 μm.

In some embodiments, provided nutraceutical compositions (e.g., particle preparations) are or comprise particles with an average diameter (e.g., D[3,2], D[4,3], etc.) of particles in the range of about 1-3000 μm, about 1-2000 μm, about 1-1000 μm, about 1-500 μm, about 1-250 μm, about 1-175 μm, about 1-100 μm, about 1-50 μm, about 1-10 μm, or about 4-6 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 1 μm to about 200 μm. In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 1 μm to about 50 μm. In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 20 μm to about 30 μm. In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 30 μm to about 40 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 50 μm to about 150 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 100 μm to about 200 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 5 μm to about 100 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 5 μm to about 175 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 1 μm to about 10 μm.

In some instances, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 1 μm to about 5 μm. In some instance, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 4 μm to about 6 μm. In some instance, particle preparations (e.g., nutraceutical compositions) comprise particles (e.g., polymer microparticles comprising a nutraceutical payload component) characterized by an average particle diameter (e.g., D[3,2], D[4,3], etc.) within a range of about 5 μm to about 10 μm.

In many embodiments, particle preparations as described and/or utilized herein are characterized in that they are substantially free of residual solvent. In some embodiment, such substantially-solvent-free character is achieved without specific solvent removal step(s).

Typically, a particle preparation is considered to be “solvent free” (e.g., “residual solvent free”) if no residual solvent (e.g., no organic solvent) is detected in the preparation above a level of about 1 ppm, about 5 ppm, about 10 ppm, about 20 ppm, about 30 ppm, about 40 ppm, about 50 ppm, about 60 ppm, about 70 ppm, about 80 ppm, about 90 ppm, about 100 ppm, about 200 ppm, about 300 ppm, about 400 ppm, about 500 ppm, about 1000 ppm, about 2000 ppm, about 3000 ppm, or about 4000 ppm.

In some embodiments, particle preparations described and/or utilized disclosed herein comprise residual solvent (e.g., low residual solvent). In some cases, a residual solvent is an organic solvent. In some embodiments, a residual solvent may be or comprise, for example, hexane, ethanol, ethyl acetate, acetone, methylene chloride, methanol, dichloromethane, isopropyl alcohol (i.e., 2-propanol), or a combination thereof.

In some cases, residual solvent content of a particle preparation is less than about 4000 ppm, about 3000 ppm, about 2000 ppm, about 1000 ppm, about 900 ppm, about 800 ppm, about 700 ppm, about 600 μm, about 500 ppm, about 400 ppm, about 300 ppm, about 200 ppm, about 100 ppm, about 90 ppm, about 80 ppm, about 70 ppm, about 60 ppm, about 50 ppm, about 40 ppm, about 30 ppm, about 20 ppm, or about 10 ppm.

In some instances, a residual solvent is or comprises dichloromethane, and a particle preparation comprises less than about 5 ppm of the dichloromethane.

In some instances, a residual solvent is or comprises hexane, and a particle preparation comprises less than about 50 ppm of hexane.

In some instances, a residual solvent is or comprises isopropyl alcohol (2-propanol), and a particle preparation comprises less than about 50 ppm of isopropyl alcohol.

In some instances, a residual solvent is or comprises ethanol, and a particle preparation comprises less than about 50 ppm of ethanol.

In some instances, residual solvent is or comprises methanol, and a particle preparation comprises less than about 50 ppm of methanol.

In some instances, residual solvent is or comprises ethyl acetate, and a particle preparation less than about 50 ppm of ethyl acetate.

In some instances, residual solvent is or comprises acetone, and a particle preparation comprises less than about 50 ppm of acetone.

In some embodiments, a particle preparation is characterized in having a water activity of less than about 0.3, about 0.25, about 0.2, about 0.15, about 0.1, about 0.09, about 0.08, about 0.07, about 0.06, about 0.05, about 0.04, about 0.03, about 0.02, or about 0.01.

### Polymer Components

Typically, as described herein, utilized polymer component(s) is or are characterized by pH-responsiveness (i.e., is a pH-responsive polymer component). In some instances, polymer component(s) is or are characterized by temperature-responsiveness (i.e., is a temperature-responsive polymer component).

As provided herein, a polymer component may be or comprises at least one polymer. In some instances, polymer component can be a combination of polymers, each of which may or may not be individually pH-responsive and/or temperature-responsive.

In some instances, a polymer component may be or comprise one or more cationic polymers, anionic polymers, zwitterionic polymers, nonionic polymers comprising a pH-labile group, or combinations thereof.

In some instances, an anionic polymer may comprise acidic groups. For example, in some embodiments, anionic polymers may comprise carboxylic acids (—COOH), sulfonic acids (—SO₃H), phosphonic acids, or boronic acids. For example, anionic polymer(s) may be polymethyl methacrylate and/or cellulose acetate phthalate.

In some instances, a polymer component may be or comprises a copolymer comprising methacrylate. For example, a polymer component (e.g., a pH-responsive polymer component) may comprise butyl methacrylate, 2-dimethylaminoethyl methacrylate, methyl methacrylate. In some instances, a polymer component may be or comprises poly(butylmethacrylate-co-(2-dimethylaminoethyl) methacrylate-co-methylmethacrylate).

In some instances, a polymer component may be or comprises polygalactomannan (e.g., guar gum). In some instances, a polymer component may be or comprises a polysaccharide (e.g., chitosan). In some instances, a polymer component may be or comprises hyaluronic acid, alginic acid, chitosan, or dextran.

In some cases, one or more pH-responsive polymer component and/or temperature-responsive polymer components is or are associated (e.g., complexed) with one or more nutraceuticals in a particle preparation (e.g., nutraceutical composition) as described herein.

### Payload Components

In some embodiments, a payload component utilized in accordance with the present disclosure is or comprises nutraceutical (i.e., is a nutraceutical payload component). In some embodiments, a payload component is or comprises at least one antioxidants, macronutrients, micronutrients, minerals, prebiotics, probiotics, prebiotics, vitamins, or combinations thereof. In some embodiments, payload component is or comprises one or more carotenoid compounds. In some embodiments, payload component is or comprises lutein, zeaxanthin, or a combination thereof. In some embodiments, payload component is or comprises vitamin D.

In some instances, a payload component (e.g., nutraceutical payload component) is fat soluble. In some instances, a payload component (e.g., nutraceutical payload component) is water soluble. In some embodiments, a payload component (e.g., nutraceutical payload component) is both fat soluble and water soluble. In some instances, a payload component (e.g., nutraceutical payload component) is partially fat soluble. In some instances, a payload component (e.g., nutraceutical payload component) is partially water soluble. In some embodiments, a payload component (e.g., nutraceutical payload component) is both partially fat soluble and partially water soluble.

Those skilled in the art are aware that many carotenoid compounds are natural products that act as antioxidants and that accumulate in the lens and retina of the eye where they protect against reactive oxygen species, blue light, and lipid peroxide damage. Cataracts and macular degeneration are associated with decreased lutein and zeaxanthin levels due to aging. The chemical stability and absorption of carotenoid compounds (specifically including lutein and/or zeaxanthin) from currently available nutraceutical compositions (e.g., particle preparations) is quite limited. Typically, carotenoid compounds (e.g., lutein, zeaxanthin) are unstable and prone to degradation when exposed to heat, oxygen, water, or light. In some instances, carotenoid compounds may comprise lutein, zeaxanthin, astaxanthin, adonirubin, adonixanthin, lycopene, α-carotene, β-carotene, α-lipoic acid, coenzyme q10, or a combination thereof.

In some embodiments, a payload component is or comprises at least one carotenoid compound (e.g., lutein, zeaxanthin, or a combination of both). In some embodiments, a payload component is or comprises lutein. In some embodiments, a payload component is or comprises zeaxanthin. In some embodiments, a payload component is or comprises both lutein and zeaxanthin.

In some cases, a payload component is or comprises at least one micronutrient. In some instances, a micronutrient is or comprises at least one vitamin. For example, a vitamin is or comprises vitamin B1, vitamin B2, vitamin B3, vitamin B5, vitamin B6, vitamin B7, vitamin B9, vitamin B12, vitamin A, vitamin C, vitamin D, vitamin E, vitamin K, or a combination thereof. In some instances, payload component is or comprises vitamin D.

In some cases, a payload component is or comprises at least one macronutrient. In some instances, a macronutrient is or comprises at least one carbohydrate, at least one fat, at least one protein, or a combination thereof.

In some cases, a payload component is or comprises at least one mineral. In some instances, a mineral is or comprises iron, zinc, calcium, magnesium, manganese, phosphorus, cobalt, potassium, sodium, chloride, iodine, sulfur, copper, fluoride, selenium, or a combination thereof.

In some cases, a payload component is or comprises at least one short chain fatty acid. In some instances, a short chain fatty acid is or comprises acetate, propionate and butyrate, or a combination thereof.

In some cases, a payload component is or comprises at least one probiotic species. In some instances, a probiotic is or comprises at least one species of yeast, at least one species of fungus, at least one species of bacteria, or a combination thereof.

In some instances, a payload component is or comprises at least one probiotic species. In some instances, a probiotic is or comprises at least one species of bacteria. In some instances, at least one species of bacteria is or comprises Lactobacillus acidophilus, Lactobacillus bulgarius, Lactobacillus rhamnosus, Lactobacillus reuteri, Streptococcus thermophilus, Saccharomyces boulardii, Bifidobacterium animalis, Bifidobacterium bifidum, Bifidobacterium lactis, Bacillus subtilis, or a combination thereof.

In some cases, a payload component is or comprises at least one prebiotic. In some instances, at least one prebiotic is or comprises non-digestible fibers (e.g., inulin), bacteriophage, or a combination thereof.

In some embodiments, a payload component is or comprises at least one hydrophobic agent (e.g., a hydrophobic compound such as a carotenoid compound) and a probiotic. Indeed, the present disclosure surprisingly demonstrates feasibility of providing preparations that include both a probiotic and an agent (e.g., such as a hydrophobic agent which may be or comprise a carotenoid compound) that might commonly be expected to interfere with probiotic viability. In some embodiments, provided preparations comprise a polymer component as described herein. Regardless, however, the present disclosure demonstrates surprising feasibility and remarkable utility of low water activity preparations and/or low solvent preparations, including in provision of compositions that include both a probiotic and another agent (e.g., a hydrophobic compound such as a carotenoid compound, e.g., astaxanthin and/or lutein). Among other things, the present disclosure specifically provides particle preparations (e.g., low solvent and/or low water content) that include a polymer component that is or comprises a pH responsive polymer and a payload component that is or comprises a carotenoid compound (e.g., astaxanthin and/or lutein) and a probiotic. In some embodiments, the present disclosure provides particle preparations (e.g., low solvent and/or low water activity preparations) that include BMC, lutein and/or zeaxanthin, and a probiotic.

In some instances, a payload component is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., nutraceutical composition).

In some cases, a payload component is or comprises a carotenoid compound (e.g., lutein, zeaxanthin, or a combination thereof). In some cases a carotenoid compound is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., nutraceutical composition).

In some cases, a payload component is or comprises lutein. In some cases lutein is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., nutraceutical composition).

In some cases, a payload component is or comprises zeaxanthin. In some cases zeaxanthin is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (e.g., nutraceutical composition).

In some instances, a payload component comprises both lutein and zeaxanthin. In some cases lutein and zeaxanthin, together, make up at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (e.g., nutraceutical composition).

In some cases, the weight ratio between lutein and zeaxanthin in a nutraceutical composition (e.g., that is or comprises a particle preparation) is in the range of about 10 to about 9, about 9 to about 8, about 8 to about 7, about 7 to about 6, about 6 to about 5, about 5 to about 4, about 4 to about 3, about 3 to about 2, about 2 to about 1, about 1.9 to about 1.8, about 1.8 to about 1.7, about 1.7 to about 1.6, about 1.6 to about 1.5, about 1.5 to about 1.4, about 1.4 to about 1.3, about 1.3 to about 1.2, about 1.2 to about 1.1, about 1.1 to about 1.0, about 1.0 to about 0.9, about 0.9 to about 0.8, about 0.8 to about 0.7, about 0.7 to about 0.6, about 0.6 to about 0.5, about 0.5 to about 0.4, about 0.4 to about 0.3, about 0.3 to about 0.2, about 0.2 to about 0.1.

In some cases, a payload component is or comprises vitamin D. In some cases vitamin D is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (e.g., nutraceutical composition).

It is contemplated that, in some embodiments, probiotics can be encapsulated inside particles in a particle preparation as described herein. Alternatively or additionally, one or more probiotics can be combined with a particle preparation as described herein (e.g., where particles of the preparation include a nutraceutical such as for example, a carotenoid compound (e.g., lutein, zeaxanthin, or a combination thereof).

In some cases, at least one probiotic species is at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., nutraceutical composition).

### Excipient Components

In some embodiments, a particle preparation (e.g., nutraceutical composition) may further comprise an excipient component. In some embodiments, an excipient component utilized in accordance with the present disclosure is or comprises components that are not payload components and are not polymer components. In some embodiments, an excipient component is or comprises at least one anti-caking component, anti-agglomerating component, anti-clumping component, anti-aggregating component, a surfactant component, a plasticizing component, or a combination thereof. In some embodiments, an excipient component is or comprises one or more starch, cellulose, and/or sugar compounds.

In some cases, an excipient component is or comprises at least one starch (e.g., Dry-Flo®), one cellulose (e.g., microcrystalline cellulose), or one sugar (maltodextrin). In some instances, an excipient component can comprise multiple excipients and combinations thereof.

In some cases, an excipient component is at least about 99 wt %, at least about 90 wt %, at least about 85 wt %, at least about 80 wt %, at least about 75 wt %, at least about 70 wt %, at least about 65 wt %, at least about 60 wt %, at least about 55 wt %, at least about 50 wt %, at least about 45 wt %, at least about 40 wt %, at least about 35 wt %, at least about 30 wt %, at least about 25 wt %, at least about 20 wt %, at least about 15 wt %, at least about 10 wt %, at least about 5 wt %, at least about 1 wt %, at least about 0.8 wt %, at least about 0.5 wt %, at least about 0.1 wt % of a particle preparation (i.e., a nutraceutical composition).

In some cases, an excipient component can lower water activity of particle preparations.

In some cases, an excipient component can lower residual solvent content of particle preparations.

In some cases, an excipient component can affect pH-responsiveness and alter release profile.

In some cases, an excipient component can affect temperature-responsiveness and alter glass transition temperatures.

In some cases, an excipient component can affect stability in water, against light, in milk, or at elevated temperatures.

### Payload Component Protection

In some embodiments, particle preparations (e.g., nutraceutical compositions) provide protection against degradation (e.g., oxidation, hydrolysis, isomerization, fragmentation, or a combination thereof) of payload component (e.g., nutraceutical payload component). In some embodiments, particle preparations (e.g., nutraceutical compositions) provide for protection against light-induced degradation of payload component (e.g., nutraceutical payload component). In some embodiments, particle preparations (e.g., nutraceutical compositions) provide for protection against heat-induced degradation of payload component (e.g., nutraceutical payload component). In some embodiments, particle preparations (e.g., nutraceutical compositions) provide for protection against water-induced degradation of payload component (e.g., nutraceutical payload component).

In some embodiments, particle preparations (e.g., nutraceutical compositions) provide for protection against degradation payload component (e.g., nutraceutical payload component) for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 24 months at ambient temperature.

In some embodiments, particle preparations disclosed herein are effective to protect against permeation of fluids (e.g., aqueous liquids, water, dairy, milk).

In some embodiments, a provided particle preparation (e.g., nutraceutical composition) is stable in that chemical integrity of a majority of a payload component it includes is maintained after passage of a period of time (e.g., at least about 1, 2, 3, 4, 5, 6, 7, or 8 weeks) under a particular environmental condition (e.g., ambient temperature). In some embodiments, chemical integrity of at least about 70%, at least about 75%, at least about 80%, at least about 85%, at least about 90%, at least about 95% or more of a payload component is maintained over the period of time under the environmental condition. In some embodiments, the period of time is up to about 8 weeks and the environmental condition is or comprises ambient temperature. In some embodiments, the period of time is up to about 2 weeks and the environmental condition is or comprises presence of water (e.g., in aqueous solution). In some embodiments, the period of time is up to about 72 hours and the environmental condition is or comprises exposure to light at elevated temperatures (e.g., about 37° C.); in some such embodiments, at least about 80%, at least about 85%, at least about 90%, or at least about 95% or more of a payload component retains its integrity over the period of time under the environmental condition.

In some embodiments, the present disclosure provides a composition including a probiotic (e.g., as a powder). In some such embodiments, a provided composition further comprises a nutraceutical such as a carotenoid compound and/or a vitamin. In some such embodiments, a nutraceutical such as a carotenoid compound and/or a vitamin is included as part of a particle preparation (e.g., a preparation of particles comprised of a polymer component—e.g., that is or comprises a pH-responsive polymer component—and a nutraceutical component); in some such embodiments, such particle preparation is homogenously mixed with the probiotic.

In some embodiments, probiotic viability is stable in a provided composition (e.g., as described above), e.g., over a period of time at a particular environmental condition. In some embodiments, viability is assessed after 6 months at ambient temperature. In some such embodiments, probiotic viability is >99.99%, >95%, >90%, >85%, or >80%.

### Payload Component Release

In some embodiments, particle preparations (e.g., nutraceutical compositions) disclosed herein provide for controlled release of payload components.

In some instances, less than about 10%, less than about 9%, less than about 8%, less than about 7%, less than about 6%, less than about 5%, less than about 4%, less than about 3%, less than about 2%, or less than about 1% of payload component is released from particle preparations (e.g., nutraceutical compositions) after soaking in water for 2 hours at 100° C.

In some instances, less than about 10%, less than about 9%, less than about 8%, less than about 7%, less than about 6%, less than about 5%, less than about 4%, less than about 3%, less than about 2%, or less than about 1% of carotenoid compound (e.g., lutein, zeaxanthin, or a combination thereof) is released from particle preparations (e.g., nutraceutical compositions) after soaking in water for 2 hours at 100° C.

In some instances, less than about 10%, less than about 9%, less than about 8%, less than about 7%, less than about 6%, less than about 5%, less than about 4%, less than about 3%, less than about 2%, or less than about 1% of lutein, zeaxanthin, or a combination thereof is released from particle preparations (e.g., nutraceutical compositions) after soaking in water for 2 hours at 100° C.

In some instances, less than about 10%, less than about 9%, less than about 8%, less than about 7%, less than about 6%, less than about 5%, less than about 4%, less than about 3%, less than about 2%, or less than about 1% of vitamin D is released from particle preparations (e.g., nutraceutical compositions) after soaking in water for 2 hours at 100° C.

In some instances, less than about 10%, less than about 9%, less than about 8%, less than about 7%, less than about 6%, less than about 5%, less than about 4%, less than about 3%, less than about 2%, less than about 1%, less than about 0.5%, less than about 0.1%, less than about 0.05%, or less than about 0.01% of payload component (e.g., nutraceutical payload component) is released from particle preparations (e.g., nutraceutical compositions) after soaking in water for 2 hours at 25° C.

In some instances, less than about 10%, less than about 9%, less than about 8%, less than about 7%, less than about 6%, less than about 5%, less than about 4%, less than about 3%, less than about 2%, less than about 1%, less than about 0.5%, less than about 0.1%, less than about 0.05%, or less than about 0.01% of carotenoid compound (e.g., lutein, zeaxanthin, or a combination of thereof) is released from particle preparations (e.g., nutraceutical compositions) after soaking in water for 2 hours at 25° C.

In some instances, less than about 10%, less than about 9%, less than about 8%, less than about 7%, less than about 6%, less than about 5%, less than about 4%, less than about 3%, less than about 2%, less than about 1%, less than about 0.5%, less than about 0.1%, less than about 0.05%, or less than about 0.01% of vitamin D is released from particle preparations (e.g., nutraceutical compositions) after soaking in water for 2 hours at 25° C.

In some embodiments, when placed in low pH solution (e.g., simulated gastric fluid, pH 1.0-2.5), greater than about 99%, greater than about 95%, greater than about 90%, greater than about 85%, greater than about 80%, greater than about 75%, greater than about 70% of payload component is released within about 15 mins.

In some embodiments, when placed in low pH solution (e.g., simulated gastric fluid, pH 2.5-4.0), greater than about 99%, greater than about 95%, greater than about 90%, greater than about 85%, greater than about 80%, greater than about 75%, greater than about 70% of payload component is released within about 15 mins.

In some embodiments, when placed in low pH solution (e.g., simulated gastric fluid, pH 4-6), greater than about 99%, greater than about 95%, greater than about 90%, greater than about 85%, greater than about 80%, greater than about 75%, greater than about 70% of payload component is released within about 15 mins.

In some embodiments, when placed in mildly acidic to mildly basic pH solution (e.g., simulated intestinal fluid, pH 6.0-8.0), greater than about 99%, greater than about 95%, greater than about 90%, greater than about 85%, greater than about 80%, greater than about 75%, greater than about 70% of payload component is released within about 15 mins.

In some embodiments, when placed in basic pH solution (e.g., alkaline water, pH 8.0-10.0), greater than about 99%, greater than about 95%, greater than about 90%, greater than about 85%, greater than about 80%, greater than about 75%, greater than about 70% of payload component is released within about 15 mins.

### Uses of Nutraceutical Compositions

It is contemplated that provided particle preparations (e.g., nutraceutical compositions) disclosed herein are suitable for use in varying consumable compositions (e.g., a food product, a beverage product, an animal-consumable product). In some instances, disclosed particle preparations (e.g., nutraceutical compositions) provide for stability of polymer component (e.g., pH-responsive polymer component), payload component (e.g., nutraceutical payload component), or a combination thereof when used with consumable compositions (e.g., a food product, a beverage product, an animal-consumable product).

Further, this disclosure provides for nutraceutical compositions (e.g., particle preparations) which may improve health. Provided technologies provide benefits over existing products because (i) in some embodiments, provided nutraceutical compositions (e.g., particle preparations) comprise low residual solvent content reducing health risks upon consumption as compared to previous technologies, and (ii) there have been no feasible technologies (e.g., cost-efficient, time-efficient, physically and/or chemically-capable) which remove and/or reduce residual solvent content to levels which reduce said health risks.

Some aspects of the current disclosure provide methods of promoting health or longevity in an animal, comprising providing an effective amount of particle preparations (e.g., nutraceutical compositions) described herein in combination with a consumable composition (e.g., a food product, a beverage product, an animal-consumable product) to an animal. In some cases, consumable compositions comprise particle preparations (e.g., nutraceutical compositions).

In some cases, an animal is a human, for example, an adult, an elder, a teenager, an adolescent, or an infant. In some cases, an animal is an agricultural animal, for example, a horse, a cow, a pig, a sheep, a goat, a domesticated bird (e.g., chicken, duck, goose), a non-domesticated (e.g., wild) bird, etc. In some cases, an animal is a pet animal, for example, a dog, a cat, a rabbit, or a fish.

Some aspects of the current disclosure provide consumable compositions (e.g., food products, beverage product, animal-consumable compositions) comprising disclosed particle preparations (e.g., nutraceutical compositions). In some cases, consumable compositions comprising particle preparations (e.g., nutraceutical compositions) is or comprises a food product. In some cases, a food product is or comprises at least one of protein bar, cereal, protein powder, salad dressing, nutrition supplement, baby formula, smoothie, yoghurt, ice cream, sachets, spice, food additive, candy, sprinkle packet, pet food, pet feed, agricultural seed, or fertilizer. In some cases, consumable compositions comprising particle preparations (e.g., nutraceutical compositions) are provided to an animal in a mixture with a food or food ingredient.

Some aspects of the current disclosure provide non-consumable compositions that are applied for agricultural applications (e.g., agricultural seed, fertilizer). In some cases, non-consumable compositions comprising particle preparations (e.g., nutraceutical compositions) is or comprises an agricultural product for plant growth or plant nutrient delivery. In some cases, non-consumable compositions comprising particle preparations (e.g., nutraceutical compositions) are provided to seeds or plants in a mixture with a seed or fertilizer or plant ingredient.

Some aspects of the current disclosure provide consumable compositions (e.g., food products, beverages, animal-consumable compositions) comprising disclosed particle preparations (e.g., nutraceutical compositions). In some cases, consumable compositions comprising particle preparations (e.g., nutraceutical compositions) is or comprises a beverage product. In some cases, a beverage product is or comprises at least one of sports drink, beer, wine, tea, coffee, milk, juice, liquid pharmaceutical formulation, or liquid supplement formulation. In some cases, the formulation is provided to an animal in a mixture with a beverage or beverage ingredient.

Some aspects of the current disclosure provide powder-based supplement, food, or beverage-mix products comprising particle preparations (e.g., nutraceutical compositions) disclosed herein. In some cases, the powder-based supplement, food, or beverage-mix products is a pre-workout powder, pre-workout capsule/pill, baby formula, whey powder, protein powder, drink powder mix (e.g., Kool-Aid type mix), or a powder-based supplement, food, or beverage-mix products.

### Exemplification

The following examples are intended to illustrate but not limit the disclosed embodiments. The following examples are useful to confirm aspects of the disclosure described above and to exemplify certain embodiments of the disclosure.

These non-limiting examples demonstrate particular features and advantages of provided technologies—e.g., of provided particle preparations in which a pH-responsive polymer component (e.g., that is or comprises a basic methacrylate copolymer) encapsulates a nutraceutical payload (e.g., lutein, zeaxanthin, and/or Vitamin D).

Among other things, provided particle preparations are characterized by significant improvements, including, for example, improved stability, controlled release, anti-caking, anti-agglomeration, anti-clumping, anti-aggregation, and/or amenability to combination with other component(s) of a product (e.g., a nutraceutical product that may in many embodiments be a consumable product). As exemplified herein, provided particle preparations achieve one or more of the following advantages: 1) Stability enhancement in water, light, and oxidative environments; 2) amenability to combination (e.g., mixing) with other components or materials, which enables payload components (specifically including carotenoid compounds and/or vitamins) to be combined with and/or incorporated into complex foods and/or beverages (e.g., milk) and/or ingredients (e.g., probiotics); 3) Low water activity, even when characterized by high water content; 4) Rapid release of payload in acidic conditions (e.g., the stomach); 5) Technological modularity that permits control over particle size characteristic(s) (e.g., average particle [e.g., microparticle] size and/or size distribution), loading, and/or release; 6) Low water activity (specifically including when provided in powder form), providing a specific advantage that preparation(s) can be mixed with probiotics without negatively impacting viability of microorganisms in such probiotics; and 7) Small size (e.g., around 5-50 microns), which facilitates or enables homogenous mixing, specifically including with a powder (e.g., probiotic powder). 8) Anti-caking, anti-agglomeration, anti-clumping, and/or anti-aggregation.

### Example 1: Morphology of Exemplary Particle Preparations

This example shows bright field and scanning electron microscopy images that document morphology of non-limiting exemplary embodiments of disclosed particle preparations as described herein that comprise certain nutraceutical payloads.

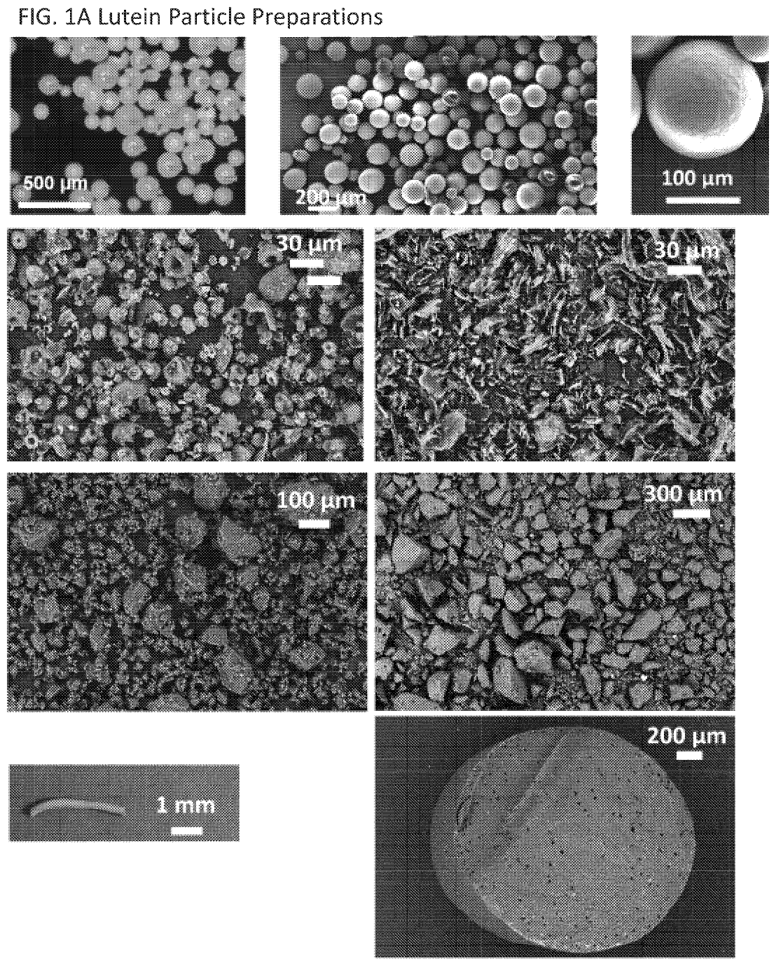

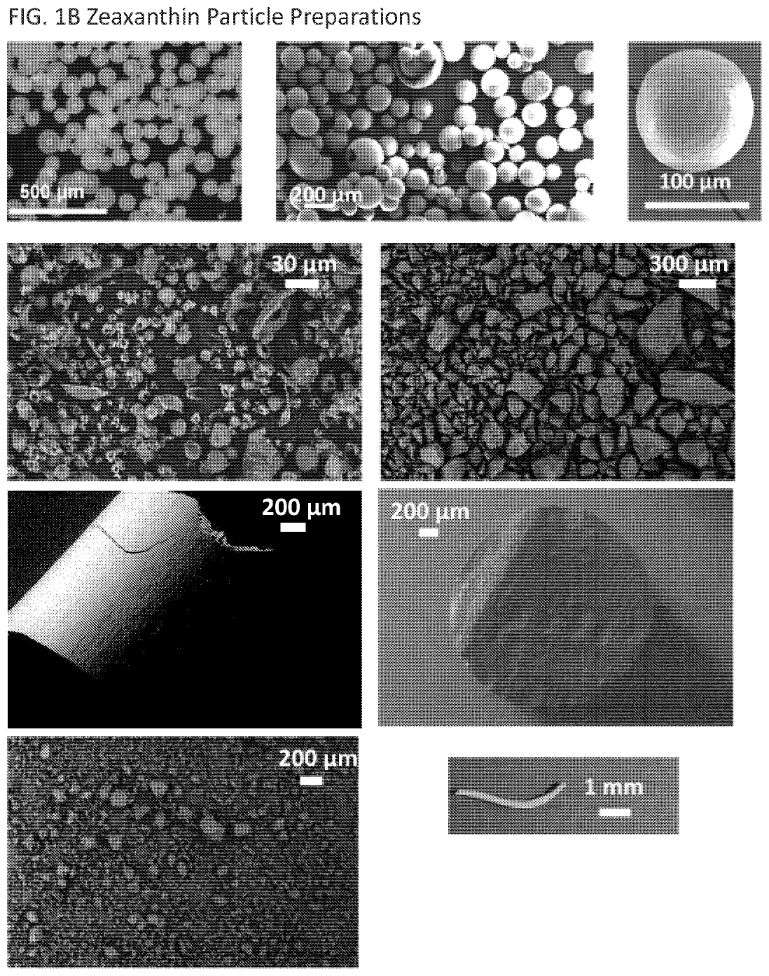

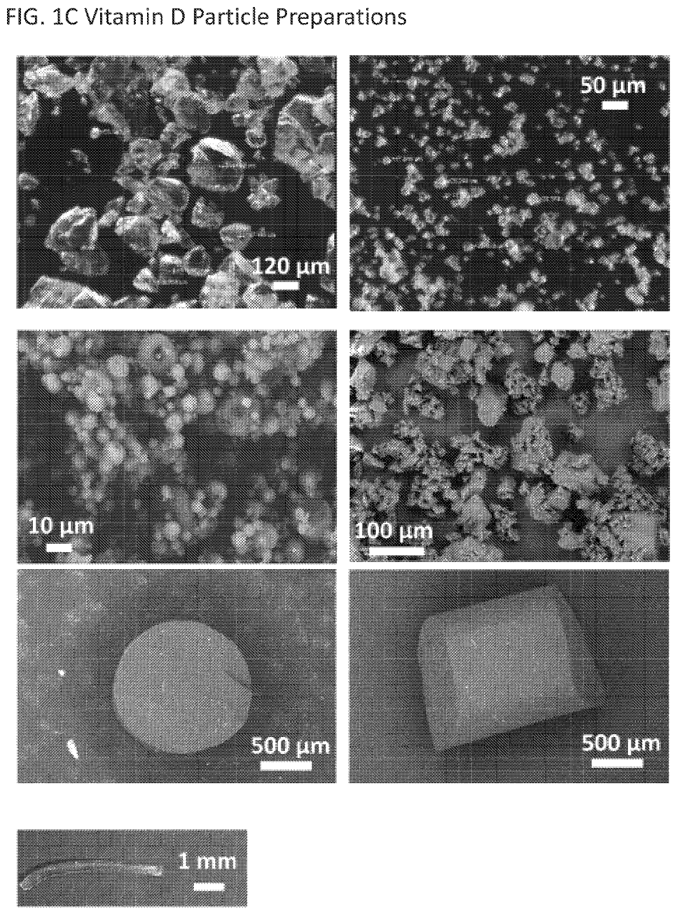

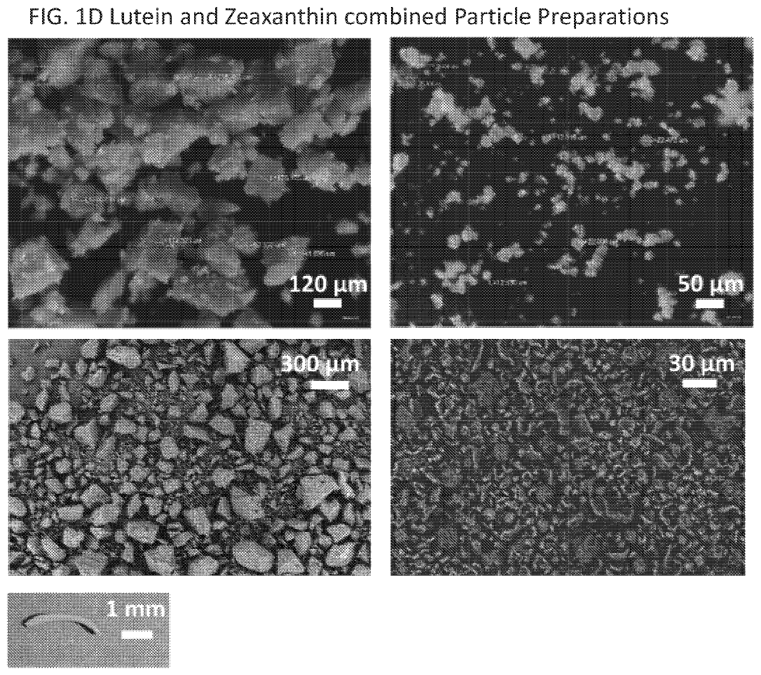

For example, FIG. 1 presents images of an exemplary payload-containing particle preparations as provided by the present disclosure. As can be seen, preparations are shown to comprise various shapes (e.g., spherical particles, donut-shaped particles, cylindrical particles, circular particles, disc-shaped particles, worm-like particles, irregular-shaped particles, etc.) of consistent size, with smooth surfaces, and bright-orange or clear color.

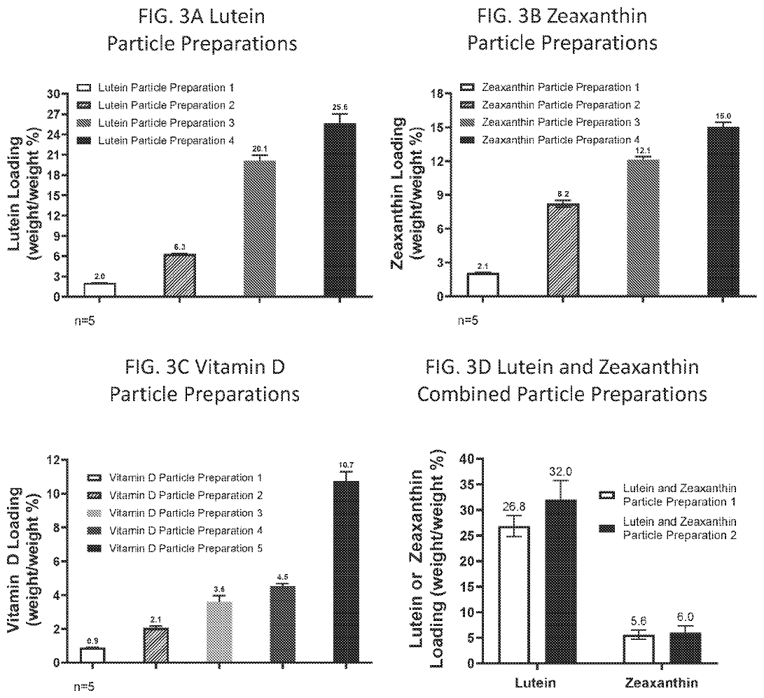

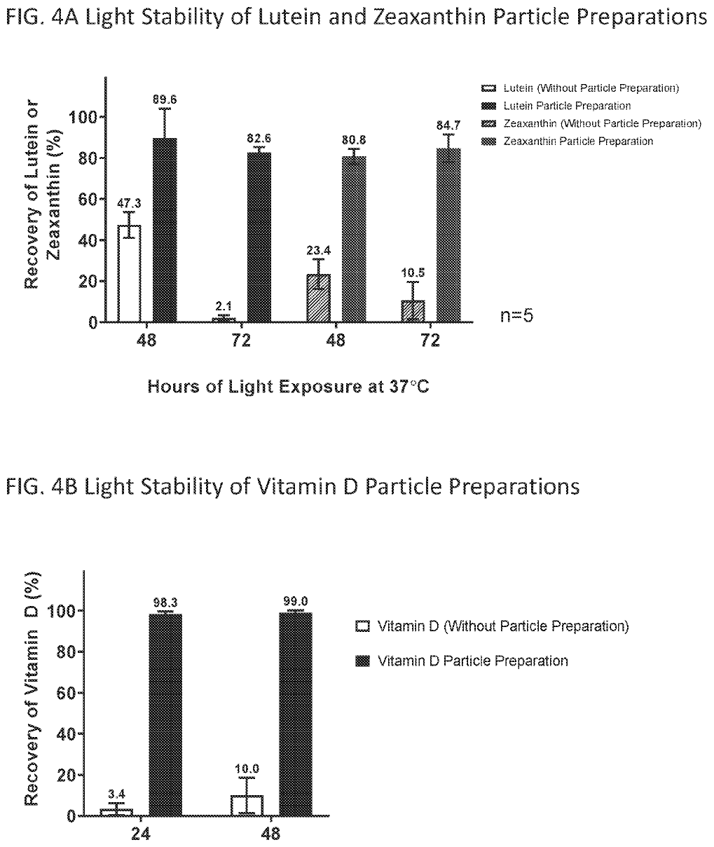

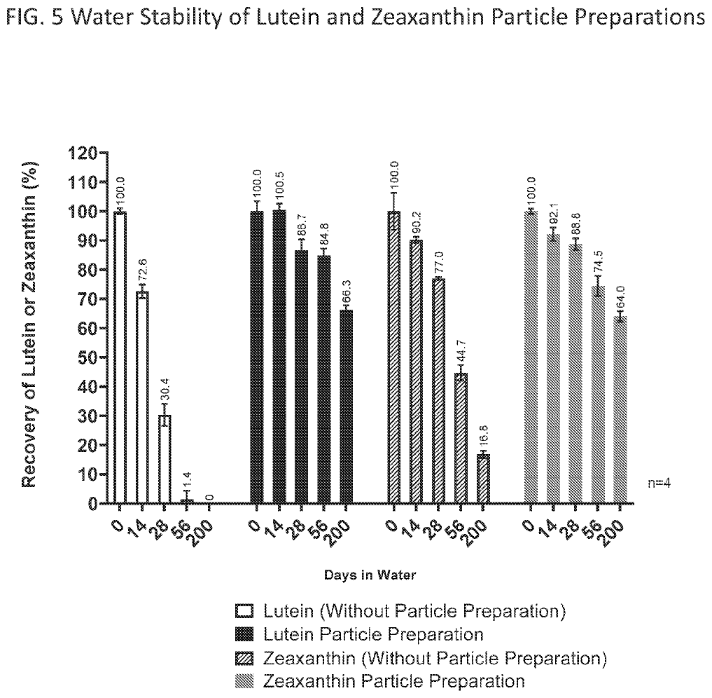

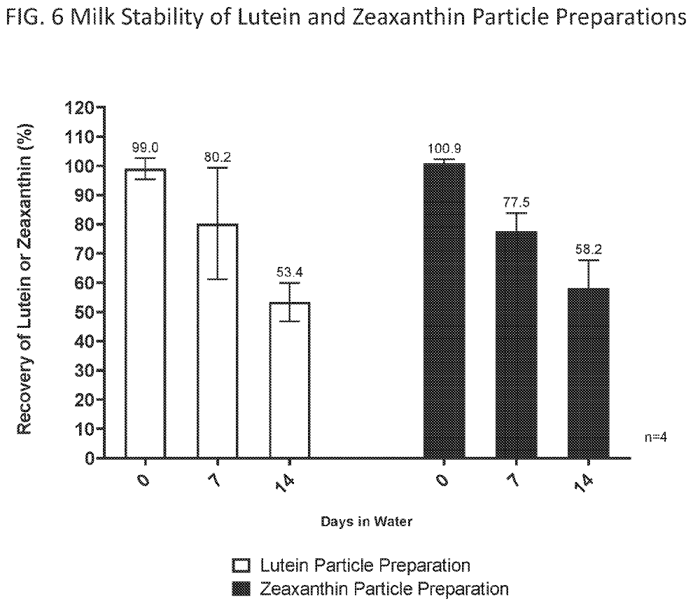

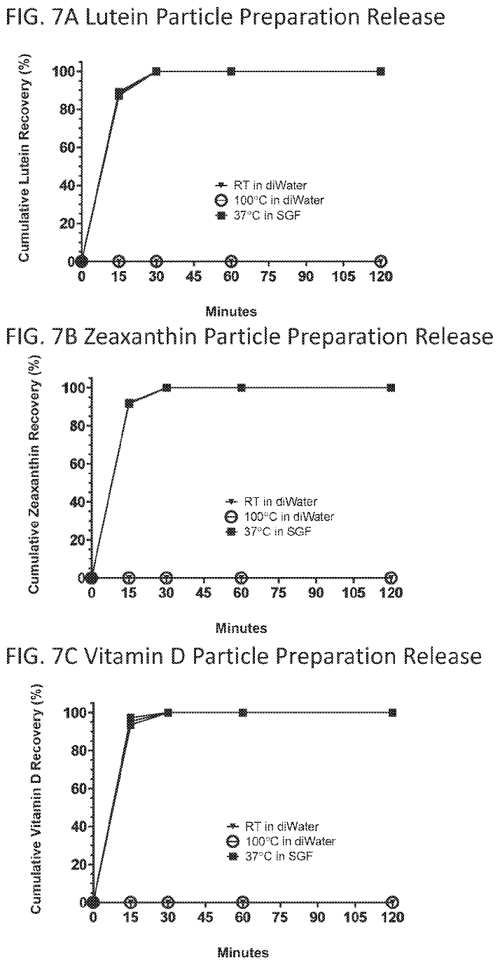

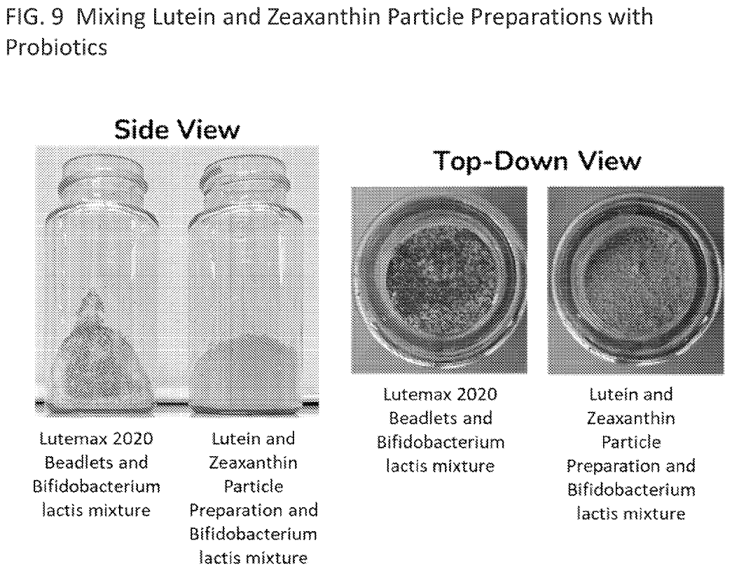

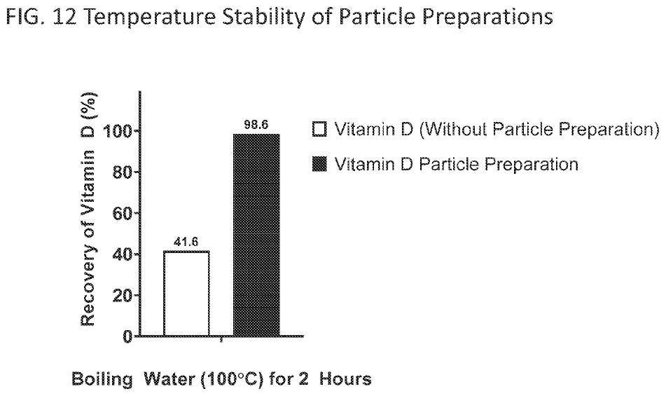

These particular exemplified particle preparations (i.e., nutraceutical compositions) utilized poly(butylmethacrylate-co-(2-dimethylaminoethyl) methacrylate-co-methylmethacrylate) (1:2:1) (also known as basic methacrylate copolymer, BMC, Eudragit E PO, Eudraguard Protect) as a polymer component. These non-limiting exemplary embodiments of particle preparations (i.e., nutraceutical compositions) demonstrate the ability to provide numerous advantages to the performance of payload (e.g., lutein and zeaxanthin), as compared to non-encapsulated payload (e.g., lutein and zeaxanthin) in terms of controlled loading (FIG. 3A-B), stability in light (over 32-fold improvement after 72 hours exposure to 82,000 lux) (FIG. 4A), stability in water (over 650-fold improvement after 200 days in water) (FIG. 5), controlled release in specific environments (release in liquid environments of less than pH 5.0) (FIG. 7A-B), prevention of release in specific environments (particles maintain encapsulated payloads in boiling water for up to 2 hours (FIG. 7A-B), and combination with other components of a product, e.g., an edible product, a probiotic (FIG. 9), milk (FIG. 6). Further, these non-limiting exemplary embodiments of particle preparations (i.e., nutraceutical compositions) provide numerous advantages to the performance of vitamin D in terms of controllable loading (FIG. 3C), stability in light (over 16-fold improvement after 24 hours exposure to 82,000 lux) (FIG. 4B), stability in boiling water (over 2-fold improvement after 2 hours in boiling water) (FIG. 12), controlled release in specific environments (release in liquid environments of less than pH 5.0) (FIG. 7C), prevention of release in specific environments (particles maintain encapsulated payloads in boiling water for up to 2 hours and in ambient water for up to 2 hours) (FIG. 7C).

### Example 2: Particle Size Distribution in Exemplary Particle Preparations

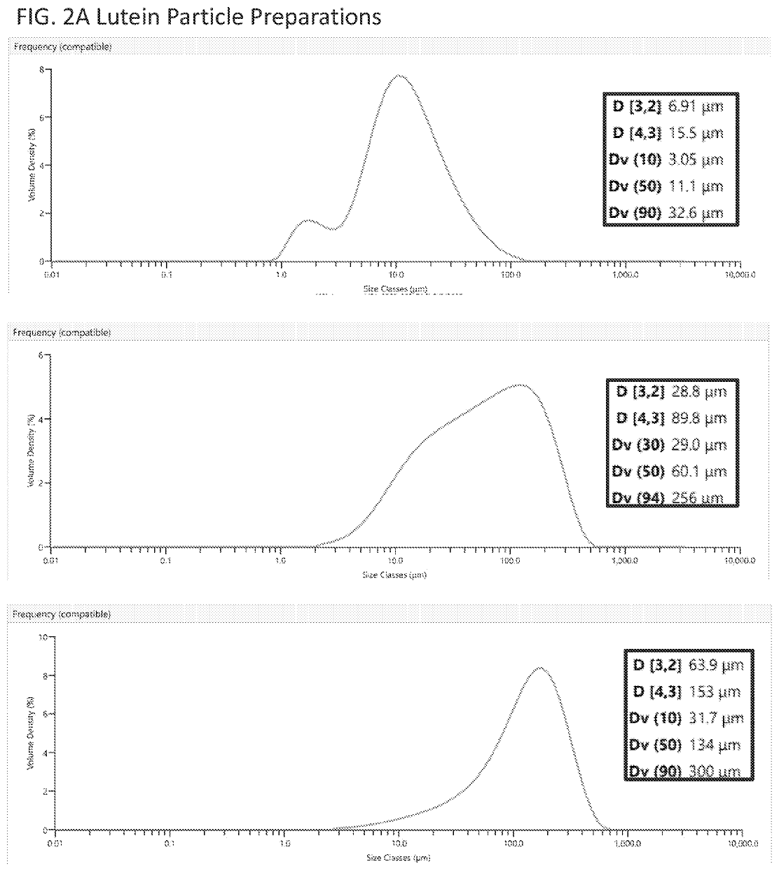

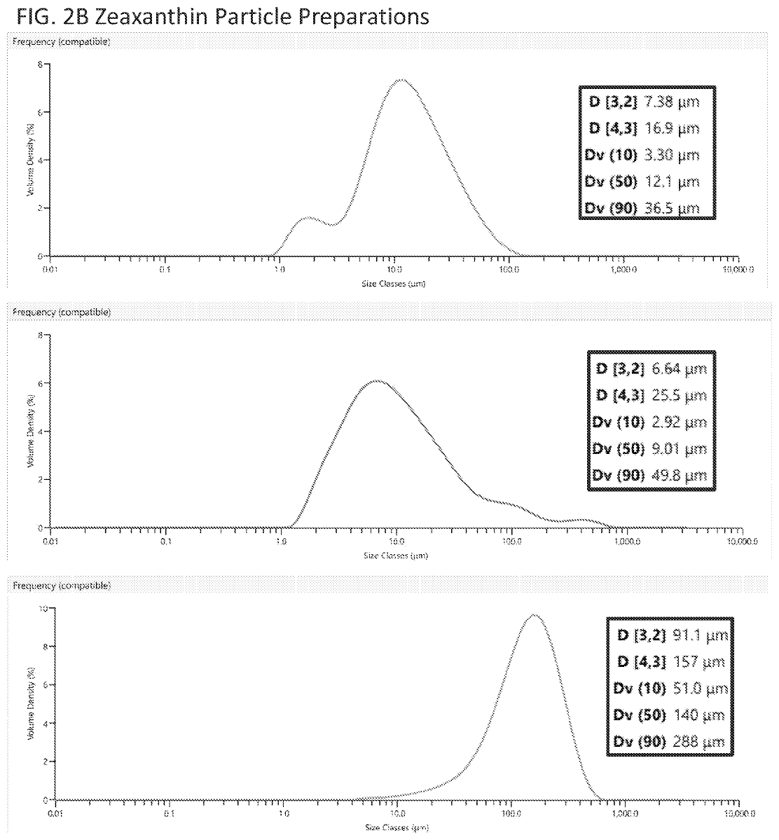

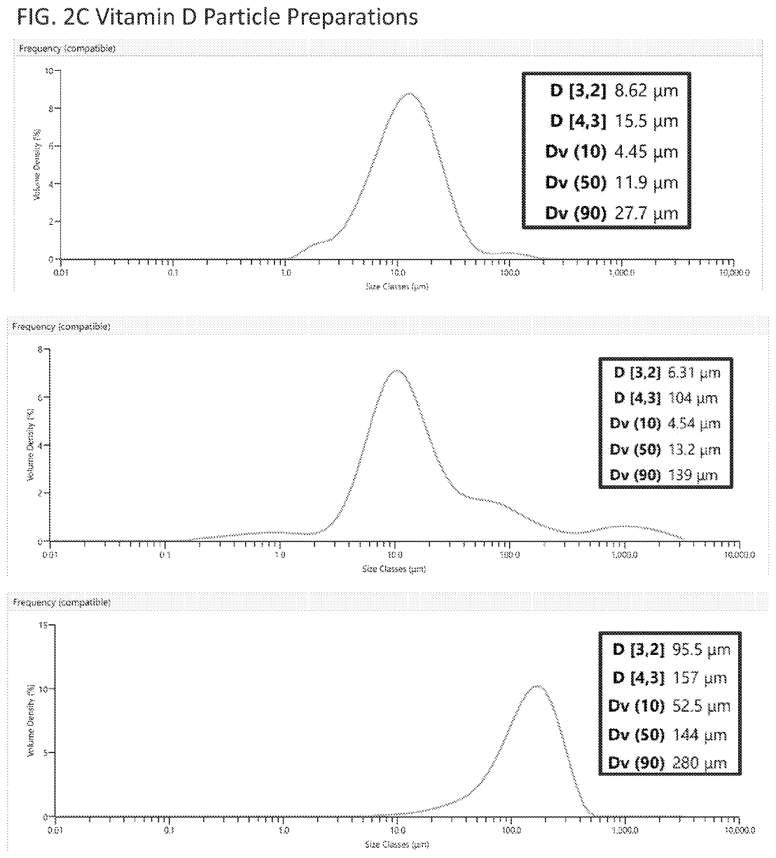

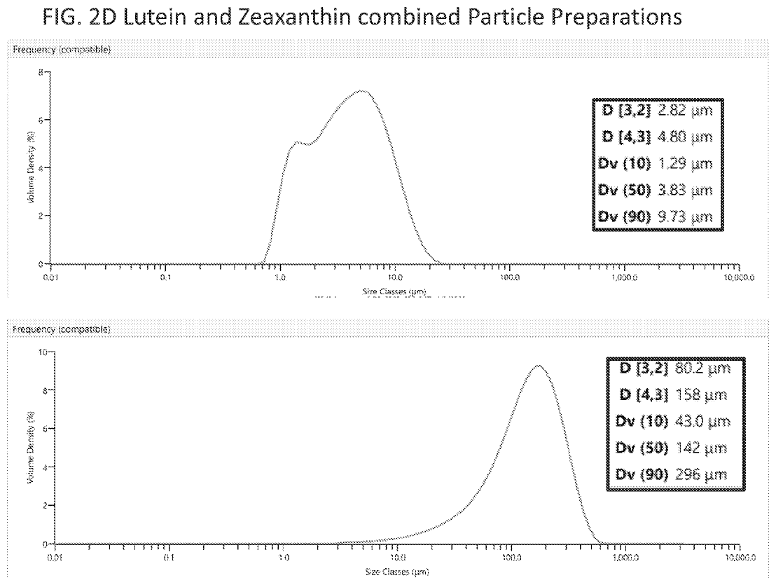

This example shows particle size distributions, as measured by a Malvern Mastersizer, of non-limiting exemplary embodiments of provided nutraceutical compositions (e.g., particle preparations comprising a nutraceutical payload), and demonstrates that relevant aspects of particle preparations can be controlled by selection of fabrication conditions. Particle size and particle size distribution influences sensory experience (e.g., mouth feel), mixing with other components of end-products (e.g., to make edible end), mixing with other constituents during formulation, and/or rate of release of payloads in nutraceutical compositions. FIGS. 2A-2D illustrate particle size distributions achieved for exemplary lutein-containing (FIG. 2A), zeaxanthin-containing (FIG. 2B), vitamin D-containing (FIG. 2C), lutein and zeaxanthin containing (FIG. 2D) preparations provided by the present disclosure. As can be seen, some of these preparations were characterized by either unimodal, bimodal, or multimodal particle size distributions, and included particles with diameters within a range of about 1 μm to about 3000 μm.

### Example 3: Payload Component Loading of Particle Preparations

This example shows that initial ratio of payload component to polymer component can be selected to control percent loading of polymer component into a provided particle preparation.

For example, FIGS. 3A-C present plots of actual loading achieved with exemplary particle preparations as described herein with lutein (FIG. 3A), zeaxanthin (FIG. 3B), Vitamin D (FIG. 3C), or lutein and zeaxanthin combined (FIG. 3D) payloads and demonstrates that payload concentration can be controlled, for example, by selecting or adjusting the ratio of initial payload component to initial polymer component (e.g., ratio by weight, ratio by volume), of which desirable ratios may be as low as 0.1% payload: 99.9% polymer component to 99.9% payload: 0.1% polymer component.

Payload component/polymer component ratio affects cost, controls payload exposure to the environment, and allows for fine-tuning of dose during manufacturing of particle preparations (i.e., nutraceutical compositions).

Data presented in FIG. 3 were generated by weighing a known amount of dried particles in glass vials, recording the exact mass of each of multiple replicate samples, capping the vials, and storing them at 4° C. in the dark overnight. After 1 night of storage in 4° C. dark, the particles were directly dissolved in 2 mL DCM to target concentration of 100 μg/mL. About 1 mL of that sample was filtered through a 0.2 μm PTFE filter and transferred into a 2 mL glass HPLC vial for HPLC analysis. Actual loading percent was calculated based on the exact mass of particles used and the actual concentration of payload was measured by HPLC. Specifically, material was weighed, then dissolved and diluted in DCM or other solvent to the following concentrations: 2 mg/mL, 200 μg/mL, 100 μg/mL, 50 μg/mL, 25 μg/mL, 12.5 μg/mL, 6.25 μg/mL, 3.125 μg/mL, 1.56 μg/mL, and 0.78 μg/mL to create a standard curve for HPLC analysis. An Agilent Infinity Lab LC/MSD iQ or Agilent Infinity 1290 is used with a Phenomenex Gemini® 3 μm C18 110 Å, LC Column 50×2 mm column. A sample diluent (Methylene chloride) with an HPLC Mobile Phase consists of 99:1 ratio of Acetonitril+0.1% formic acid: water with a flow rate of 1 mL/min and an injection volume of 2-3 μL is used. When quantifying vitamin D as nutraceutical, UV absorbance wavelength of 260 nm* is used with a retention time of about 2.6-2.9 min* at a run time of 4 minutes+1 minute post-run time. When quantifying lutein as nutraceutical, a UV absorbance wavelength of 450 nm is used with retention times of about 1.9-2 min at a run time of 4 minutes+1 minute post-run time. When quantifying zeaxanthin as nutraceutical, a UV absorbance wavelength of 450 nm is used with retention times of about 2.1 minutes at a run time of 4 minutes+1 minute post-run time.

### Example 4: Light Stability of Payload Components in Particle Preparations

This example illustrates ability of provided particle preparations to increase light stability of a payload component included therein.

For example FIG. 4A demonstrates dramatic increases in stability of lutein (about 32-fold) and zeaxanthin (about 8-fold) when exposed to light while incorporated into a provided particle composition as compared with in its “free” form. FIG. 4B documents an analogous dramatic increase (over 15-fold) in light stability for vitamin D achieved by incorporation into a provided particle preparation.

Those skilled in the art will appreciate that many carotenoid compounds, specifically including lutein and zeaxanthin, are notoriously unstable when exposed to light. Ability of provided technologies to improve light stability represents a significant technical advantage that, among other things, can improve shelf-life, shelf-storage, etc.

As demonstrated by the non-limiting exemplary nutraceutical compositions described and analyzed in the present example, the present disclosure provides particle preparations characterized by high payload component stability (e.g., 65% chemical stability). That is, among other things, the present disclosure provides nutraceutical compositions that are or comprise particle preparations in which a payload component is protected by association with a polymer component (e.g., a pH-responsive polymer component) and is characterized in that, when exposed to light for a period of time, maintains at least about 65% of intact payload component compared with that present at the beginning of the period of time (e.g., prior to the exposure to light).

In some embodiments, light stability of a payload component (e.g., a nutraceutical compound) is assessed by weighing an amount of material including the payload component (e.g., of a dry particle preparation as described herein) in a glass vial and recording the mass of each of multiple replicate samples. Vials are then placed uncapped under a light bulb measuring 85,000 lux for a period of time between about 24 to about 72 hours. Temperature readings are also recorded throughout the experiment to be about 37° C. After 24 hours, the vials containing dry particles are dissolved in 2 mL DCM to concentration of 100 μg/mL. About 1 mL of that sample is filtered through a 0.2 μm PTFE filter and transferred into a 2 mL glass HPLC vial for HPLC analysis as described in Example 3.

### Example 5: Water Stability of Payload Components in Particle Preparations

This example illustrates ability of provided particle preparations to increase water stability of a payload component included therein.

For example, FIG. 5 demonstrates significant increases in stability for lutein (650-fold) and zeaxanthin (>3.5-fold) increase in water stability (e.g., in amount recovered after a period of time—200 days in the particular study whose results are presented in FIG. 5—in water, as compared with that present when the relevant payload compound is maintained under comparable conditions in its “free” form. In the study documented in FIG. 5, stability of lutein and/or zeaxanthin were observed for about 200 days of water exposure, and showed >63% chemical stability.

Those skilled in the art will appreciate that increased water stability can improve, for example, shelf-life and shelf-storage. Furthermore, ability of provided technologies to improve water stability of included payload compounds enables such compounds to be incorporated into or included in water-based compositions (e.g., water-based food products or other edible compositions) or other materials whose water content would otherwise destroy or negatively impact the payload compound(s). Thus, the present disclosure provides water-containing compositions that include payload component(s) (e.g., nutraceutical compounds) formulated in particle compositions as described herein, specifically including where such payload component(s) are or comprise agent(s) or material(s) that are otherwise not stable to water exposure.

### Example 6: Milk Stability of Payload Components in Particle Preparations

This example illustrates ability of provided particle preparations to protect payload component(s) (specifically lutein and/or zeaxanthin when maintained in milk.

For example, FIG. 6 demonstrates stability for lutein and zeaxanthin when maintained in milk for a period of time (2 weeks in the particular study whose results are presented in FIG. 6), as compared with that observed when the relevant compound is in its “free” form. In the study documented in FIG. 6, stability of lutein and/or zeaxanthin were observed for about 2 weeks of milk exposure; each showed >50% chemical stability.

Thus, the present disclosure demonstrates that provided technologies protect payload components (e.g., nutraceutical compounds such as lutein and/or zeaxanthin) so that they can be incorporated into milk-based products (e.g., milk, cheese, yoghurt, ice cream, etc.).

### Example 7: Release of payload components from particle preparations

This example documents release characteristics of provided nutraceutical compositions that are or comprise particle preparations as described herein. Specifically, the present example documents that provided technologies achieve specific release patterns—e.g., releasing under specific environmental conditions (e.g., specific pH conditions).

FIGS. 7A-7C document that exemplary nutraceutical compositions (e.g., particle preparations comprising a nutraceutical payload) release payload component (i.e. lutein in FIG. 7A, zeaxanthin in FIG. 7B, and Vitamin D in FIG. 7C), but only in low pH conditions (e.g., in simulated gastric fluid (SGF), wherein pH is about 1.2).

Thus, the present example demonstrates that provided nutraceutical compositions (e.g., that comprise a payload component associated with a pH-responsive polymer component as described herein) achieve controlled release of payload component: release at low pH (e.g., pH less than about 5), but protection (i.e., stability) at higher pH (e.g., pH greater than about 6), even at high temperatures (e.g., 100° C.).

Advantages achieved by provided technologies include minimal release of payload component in water at ambient temperature or at a temperature of about 100° C. For example, see FIG. 7A, which documents minimal release (e.g., less than about 5%) of lutein (payload component) from an exemplary provided particle preparation (i.e., nutraceutical composition) when exposed to water at ambient temperature or at a temperature of about 100° C.; FIG. 7B demonstrates comparable minimal release (e.g., less than about 5%) of zeaxanthin (payload component), and FIG. 7C demonstrates comparable results with Vitamin D (payload component), in each case when a provided particle preparation (i.e., nutraceutical composition) is exposed to water at ambient temperature or at a temperature of about 100° C.

An exemplary protocol for determining payload component release from particle preparations (i.e., nutraceutical compositions) in water at ambient temperature involves weighing an amount of particle preparation (i.e., nutraceutical composition) in glass vials. 1 mL of deionized ultrapure water is added to each vial. Vials are placed on a rotisserie, rotating at room temperature at 35 rpm. At incremented time points, a vial is removed from the rotisserie and is centrifuged for 2 minutes at 200 rpm. Then, 900 μL of the 1 mL is sampled from a time point vial and filtered through a 10 μm pore-size cell strainer and is then mixed into a 7 mL glass vial containing 5 mL of DCM. 900 μL is added back to the vials and each vial is mixed, and is returned to its testing condition for another 15 minutes. At the end of the experiment, 1 mL of DCM is added to each time point vial and the sample is prepared for HPLC analysis as described above.

An exemplary protocol for evaluating payload component release from particle preparations (i.e., nutraceutical compositions) in water at elevated temperature (e.g., 100° C.) involves weighing an amount of particle preparation (i.e., nutraceutical composition) in glass vials. Vials are placed on an Eppendorf thermo-shaker, and set to shake at 100° C. and 500 rpm. At incremented time points, a vial is removed from the rotisserie and is centrifuged for 2 minutes at 200 rpm. Then, 900 μL of the 1 mL is sampled from a time point vial and filtered through a 10 μm pore-size cell strainer and is then mixed into a 7 mL glass vial containing 5 mL of DCM. 900 μL is added back to the vials and each vial is mixed, and is returned to its testing condition for another 15 minutes. At the end of the experiment, 1 mL of DCM is added to each vial and the sample is prepared for HPLC analysis as described above.

An exemplary protocol for evaluating payload component release from nutraceutical compositions in simulated gastric conditions (e.g., in simulated gastric fluid such as in a fluid at pH about 1.2) involves weighing an amount of particle preparation (i.e., nutraceutical composition) in glass vials. 1 mL of simulated gastric fluid is added to the vials. Vials are placed in a 37° C. incubator are shaken at 100 rpm. At incremented time points, a vial is removed from the rotisserie and is centrifuged for 2 minutes at 200 rpm. Then, 900 μL of the 1 mL is sampled from a time point vial and filtered through a 10 μm pore-size cell strainer and is then mixed into a 7 mL glass vial containing 5 mL of DCM. 900 μL is added back to the vials and each vial is mixed, and is returned to its testing condition for another 15 minutes. At the end of the experiment, 1 mL of DCM is added to each vial and the sample is prepared for HPLC analysis as described above.

### Example 8: Water Activity of Provided Particle Preparations

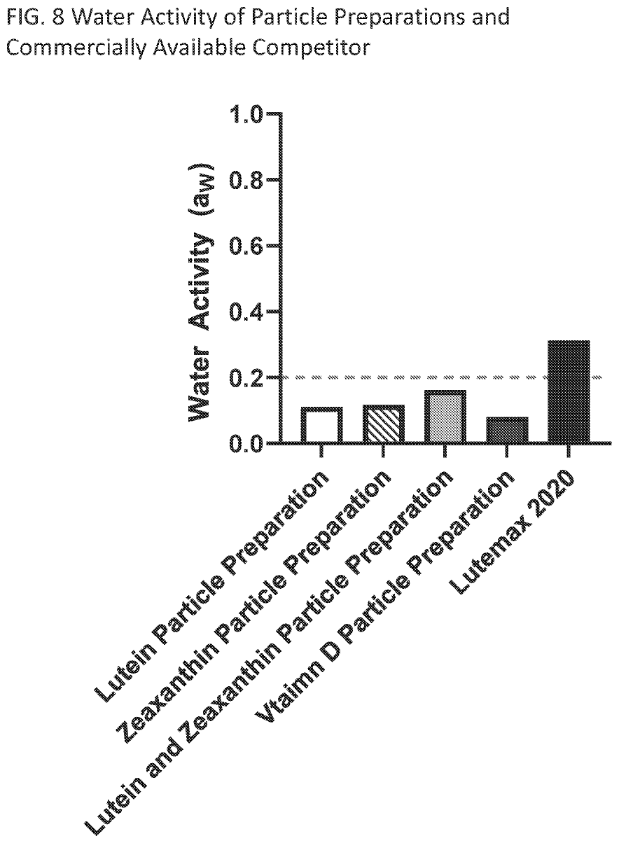

This example (FIG. 8) documents low water activity of non-limiting exemplary embodiments of provided particle preparations (i.e., provided nutraceutical compositions). Such low water activity provides a number of advantages to provide particle preparations (i.e., provided nutraceutical compositions) that are or comprise particle preparations as described herein, including that it renders them amenable to combination with probiotic agents. Indeed, the present disclosure identifies the source of a problem with various prior art nutraceutical preparations—specifically including preparations that are or comprise carotenoid compounds such as lutein and/or zeaxanthin and/or vitamin compounds such as Vitamin D—in that their high water activity can render them incompatible with certain probiotic agents. Among other things, the present disclosure provides compositions that include a probiotic agent and a nutraceutical preparation as described herein (e.g., a particle preparation comprising a payload component and a pH responsive polymer component, which particle preparation is characterized by low water activity).

Without wishing to be bound by any particular theory, the present disclosure proposes that particulate (e.g., microparticulate) character of provided compositions may mitigate detrimental interactions between payload component(s) (e.g., nutraceutical payload component[s]) and probiotic(s). Moreover, the present disclosure provides particle preparations characterized by surprisingly low water activity. Without wishing to be bound by particular theory, the present disclosure proposes that this low water activity may contribute to and/or may be required for probiotic compatibility of provided nutraceutical compositions that are or comprise such low water activity particle preparations.

For example, FIG. 8 shows non-limiting exemplary embodiments of provided nutraceutical compositions (e.g., Lutein, zeaxanthin, and vitamin D particle preparations), and demonstrates that they are characterized by low water activity, as measured by TDL2 water activity meter. Measured water activity values of these provided compositions were demonstrated to be lower than those of other product(s) (e.g., OmniActive Lutemax 2020® Beadlets) containing the same payload(s) (e.g., lutein).

It is contemplated that embodiments of provided nutraceutical compositions will help to enable storage and combination of payload component (e.g., nutraceutical payload component) with moisture-sensitive component(s) (e.g., probiotics). Again without wishing to be bound by particular theory, the present disclosure proposes that provided low water content compositions (e.g., particle compositions with a nutraceutical payload) may limit transport of water (e.g., from particle(s) to the environment), and thus may confer benefit to (e.g., may improve stability of) other component(s) or material(s) with which they are combined or otherwise associated, particularly to the extent that such other component(s) or material(s) may otherwise display sensitivity to water. In some particular embodiments, such water-sensitive component(s) or material(s) may be probiotic(s). In some embodiments, a provided particle preparation (i.e., nutraceutical composition) includes a low water activity particle preparation comprising a polymer component (e.g., a pH-responsive polymer component) and a payload component (e.g., a nutraceutical payload component) in combination with a probiotic; alternatively or additionally, in some embodiments, a provided particle preparation (i.e., nutraceutical composition) includes a low water activity particle preparation that itself includes (e.g., incorporates and/or encapsulated) a probiotic, which may confer protective benefits to other components that nutraceutical compositions (e.g., particle preparations) may comprise or be combined with and that are often sensitive to water.

### Example 9: Characteristics of Mixing Particle Preparations with Probiotics

This example shows disclosed particle preparations (i.e., nutraceutical compositions) mix homogenously with probiotic powder (e.g., Bifidobacterium lactis), as demonstrated in FIG. 9. It is contemplated that non-limiting exemplary embodiments of particle preparations (i.e., nutraceutical compositions) can be homogenously mixed with probiotics. Without wishing to be bound by any particular theory, the present disclosure proposes that size characteristics of certain provided particle preparations may surprisingly contribute desirable and/or useful attribute(s) to such preparations, specifically including, for example, amenability to homogenous combination with other component(s), specifically including powder component(s) such as probiotic powder(s).

For example, FIG. 9 demonstrates that an exemplary non-limiting particle preparation comprising particles of a small diameter (about 5 μm on average) mixes homogenously with a probiotic powder (e.g., Bifidobacterium lactis-probiotic powder), and furthermore demonstrates improved such mixing than that achieved with a commercially available product (e.g., OmniActive Lutemax 2020® Beadlets), which is shown to exhibit heterogeneous mixing with the same probiotic power (e.g., Bifidobacterium lactis probiotic powder). It is contemplated that such improved mixing behavior may help enable accurate dosing (e.g., into capsules) of particle preparations (i.e., nutraceutical compositions. Further, heterogeneous mixing of other products (e.g., OmniActive Lutemax® 2020) with probiotic powder (e.g., Bifidobacterium lactis probiotic powder) will very likely lead to manufacturing issues related to packaging, accurate dosing, and variability of product between different batches.

### Example 10: Viability of Probiotics Mixed with Particle Preparations

This example shows that provided particle preparations (i.e., nutraceutical compositions) can be combined with and/or can incorporate a probiotic without significant decrease in probiotic viability.

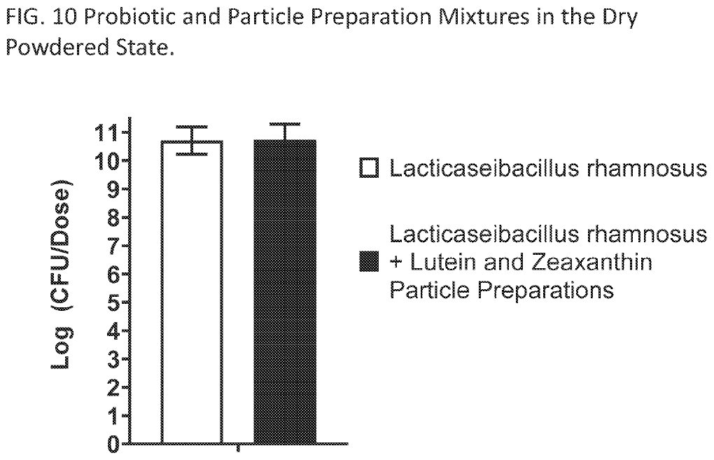

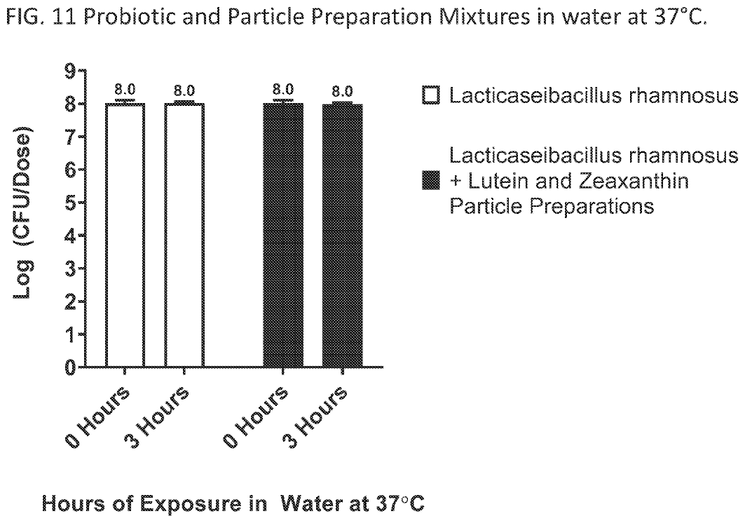

Turning to FIG. 10, 99.99% retention of probiotic viability was exhibited when non-limit exemplary embodiments of a provided particle preparation was combined with a probiotic. Thus, provided technologies achieve a variety of advantages including, for example, homogenous combination of provided particle preparations (e.g., comprising a nutraceutical payload and pH-responsive polymer component) with materials such as powder materials, including water-activity-sensitive materials such as probiotics. Among other things, the present disclosure provides nutraceutical compositions that comprise particles of a nutraceutical payload, a pH-responsive polymer component combined (e.g., homogenously mixed) with a probiotic. In some embodiments, such a composition may be or have been stored for a particular period of time and/or under particular conditions and may be characterized by one or both of nutraceutical stability and probiotic viability. In some such embodiments, the nutraceutical is or comprises a carotenoid compound such as lutein and/or zeaxanthin, and/or a vitamin such as Vitamin D. Alternatively or additionally, in some such embodiments, the storage conditions may be or comprise high temperature (e.g., up to or above about 100° C.) and/or light and/or presence of water or another aqueous liquid (e.g., milk) and/or presence of a dairy product; in some such embodiments, a stored composition maintains at least about 50% of one or more payload components in relation to the starting amount (100%) and/or at least about 109 colony forming units of probiotics. For example, in some embodiments, provided nutraceutical compositions that comprise particles of a nutraceutical payload, a pH-responsive polymer component combined (e.g., homogenously mixed) with a probiotic are characterized in that probiotic viability is maintained (as compared with that observed for “free” probiotic) when such compositions when placed in water for about 3 hours at about 37° C., as exhibited in FIG. 11. Thus, nutraceutical compositions (e.g., particle preparations comprising nutraceutical payload) presented herein present an improvement over the art in that they can further comprise probiotic and mix with water while mitigating probiotic viability loss.

### Example 11: Heat Stability of Payload Components in Particle Preparations

This example documents heat stability of payload components in non-limiting exemplary embodiments of provided particle preparations (i.e., nutraceutical compositions). Specifically, the present example (FIG. 12) demonstrates that Vitamin D is significantly (at least 2-fold) more stable at high temperatures (e.g., in boiling water, e.g., at about 100° C.) when incorporated in a particle composition as provided by the present disclosure than when in its free form. This non-limiting exemplary embodiment demonstrates a technical advantage of provided compositions given that increased heat stability, particularly when, as here, it is combined with increased water stability, can improve shelf-life, shelf-storage, etc.

An exemplary protocol for assessing heat stability as described herein can involve weighing a known amount of a particle preparation (i.e., nutraceutical composition) in a glass vial. 1 mL of deionized ultrapure water is added to the vial and mixed. This vial is placed on a heat block for 2 hours at 100° C. After 2 hours, the heat sample is allowed to cool for 5 minutes. Then, 2 mL of DCM is added to each vial and mixed. The sample is allowed time for the water and DCM layers to separate and the water phase is removed. After the layers separate, about 1 mL of the sample is filtered through a 0.2 μm PTFE syringe-filter and transferred into a 2 mL glass HPLC vial for HPLC analysis, as described above.

### Example 12: Anti-Caking/Anti-Agglomerating/Anti-Aggregating/Anti-Clumping Particle Preparations

This example illustrates ability of provided particle preparations to improve the anti-caking, anti-clumping, anti-agglomerating, anti-aggregation properties of the polymer component in the absence of any excipient (e.g., particle preparations only container polymer component and payload).

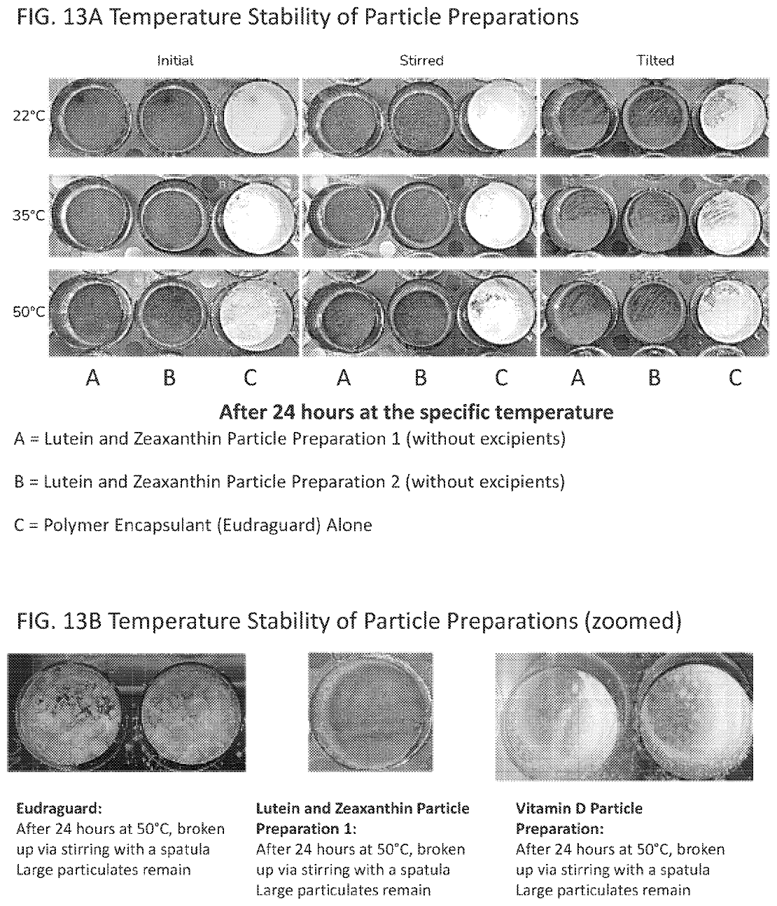

For example, FIG. 13 demonstrates lutein and zeaxanthin (FIG. 13A-B) or vitamin D (FIG. 13B) particle preparations reducing the caking/aggregation/clumping/agglomeration as compared to the polymer component along when exposed to 50° C.

Thus, the present disclosure demonstrates that provided technologies improve anti-caking, anti-clumping, anti-agglomerating, and/or anti-aggregation properties so that they can remain stable and free-flowing at higher temperatures.

### Example 13: Anti-Caking/Anti-Agglomerating/Anti-Aggregating/Anti-Clumping Particle Preparations with Excipients

This example illustrates ability of provided particle preparations to improve anti-caking, anti-clumping, anti-agglomerating, and/or anti-aggregation properties of the polymer component when excipients are added and mixed post-manufacturing.

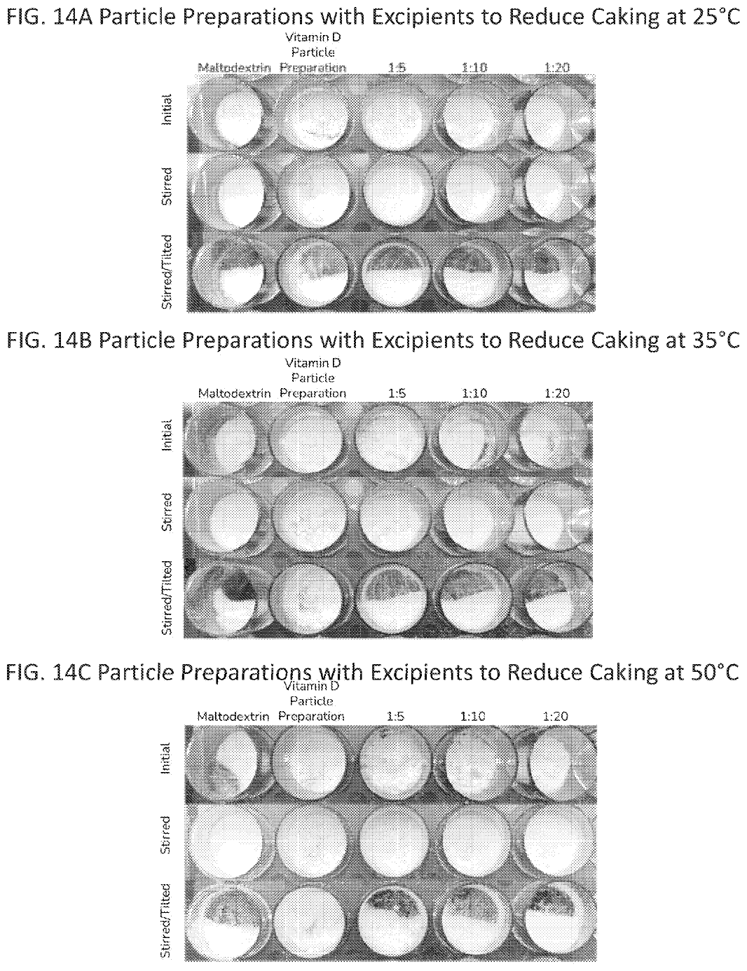

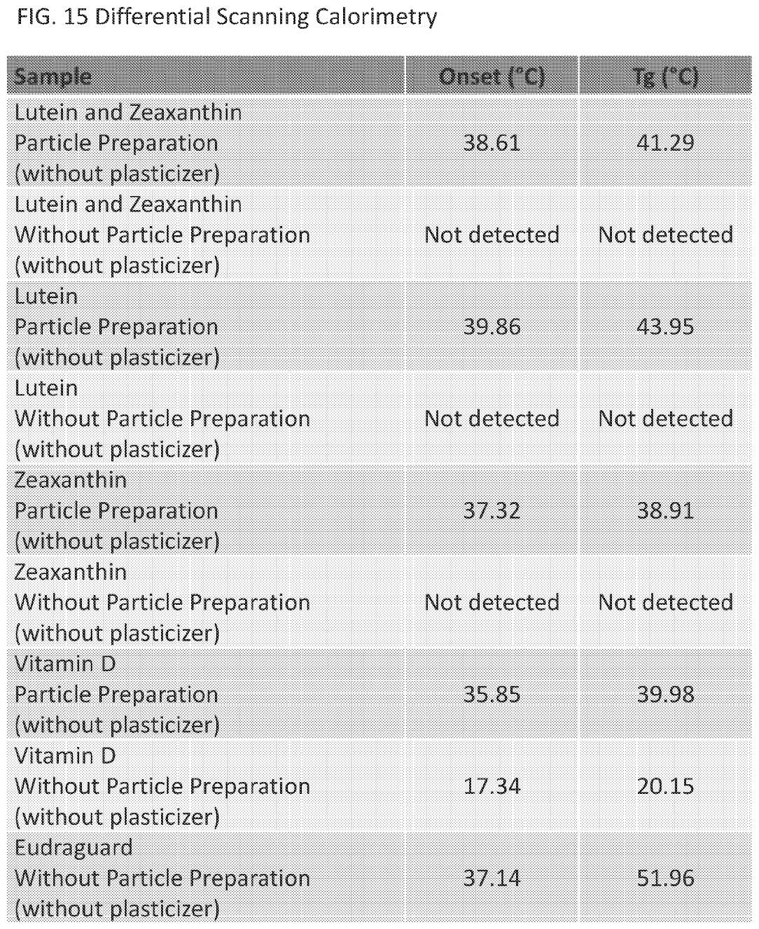

For example, FIG. 14 demonstrates vitamin D particle preparations stored at various temperatures 25° C. (FIG. 14A), 35° C. (FIG. 14B), or 50° C. (FIG. 14C) for 24 hours. Through the addition of excipients (e.g., maltodextrin) the caking/aggregation/clumping/agglomeration was reduced as compared to particle preparations without excipients.

Thus, the present disclosure demonstrates that provided technologies improve the anti-caking, anti-clumping, anti-agglomerating, anti-aggregation properties so that they can remain stable and free-flowing at higher temperatures when excipients are added post-manufacturing.

### Example 14: Payloads Improve Manufacturing by Reducing Glass Transition Temperatures

This example illustrates ability of nutraceutical payloads (e.g., lutein, zeaxanthin, etc.) to reduce the glass transition temperature of a polymer component, thereby enabling manufacturing approaches (e.g., extrusion) to proceed at lower-temperatures.

For example, FIG. 14 demonstrates vitamin D particle preparations stored at various temperatures 25° C. (FIG. 14A), 35° C. (FIG. 14B), or 50° C. (FIG. 14C) for 24 hours. Through the addition of excipients (e.g., maltodextrin) the caking/aggregation/clumping/agglomeration was reduced as compared to particle preparations without excipients.

Thus, the present disclosure demonstrates that provided technologies improve the anti-caking, anti-clumping, anti-agglomerating, anti-aggregation properties so that they can remain stable and free-flowing at higher temperatures when excipients are added post-manufacturing.

### Example 15: Preparation of Particle Preparations Via Extrusion

Preparing payload component: Optionally, the payload is micronized prior to extrusion. A DynoMill Multi-Lab bead mill is used to pre-micronize payload component with the following settings: (i) 2986 rpm, (ii) 250 mL/min pump/feed rate, and (iii) N2 purging over the beaker headspace. 500 mL deionized water is purged with N₂ for 30 minutes and is combined with: (i) 0.5 g Vit E TPGS, (ii) 50 g of a nutraceutical payload (e.g., a carotenoid compound such as lutein, zeaxanthin, etc., and (iii) 400 mL of 0.65 mm beads. The resulting slurry of payload component is dried using a lyophilizer or other drying apparatus.

Preparing particles comprising payload component: Eudraguard Protect or other polymer component is combined with the lyophilized payload component and the combination is mixed. In some embodiments, plasticizers such as soybean oil, hardened palm oil, vitamin E, or other vegetable oils are optionally added up to 10% w/w and mixed with a mechanical mixer. The resulting mixture of polymer component and payload component is extruded using a Thermo Haake Minilab II at a temperature in the range of 50° C. up to 200° C. and a screw speed between 30-90 RPM. The resulting extrudate is milled (e.g., cryo mill, jet mill, or other mill, or multiple in sequence) to produce a particle preparation (i.e., nutraceutical composition) comprising particles of 2-400 μm in size with a payload component loading of about 1-25%.

### Example 16: Preparation of Nutraceutical Compositions Via Tri-Fluid Nozzle Spray Drying

Preparing payload component: A DynoMill Multi-Lab bead mill is used to pre-micronize payload component with the following settings: (i) 2986 rpm, (ii) 250 mL/min pump/feed rate, and (iii) N2 purging over the beaker headspace. 500 mL deionized water is purged with N₂ for 30 minutes and is combined with: (i) 0.5 g Vit E TPGS, (ii) 50 g of nutraceutical (e.g., a carotenoid compound such as lutein, zeaxanthin, etc. and/or a vitamin such as vitamin D, etc), and (iii) 400 mL of 0.65 mm beads. In some instances, the combination is milled for 60 min, adding additional deionized water as needed to maintain suitable viscosity.

Preparing particles comprising payload component: Acid, base, and/or surfactant is optionally used to dissolve the polymer component. A surfactant fluid is prepared by dissolving 0.1 g of surfactant (e.g., sodium dodecyl sulfate) into 150 g water at room temperature. A polymer solution is prepared by adding 3.68 g of 6N sulfuric acid (1.01 g sulfuric acid, 2.66 g water) to 10 g of Eudraguard Protect or other polymer component and stirring up to 1000 rpm for up to 2 hours until the polymer solution is clarified with the surfactant fluid. Then, 1-10 g of slurry is added to the polymer solution and is homogenized at 15000 rpm. A basic fluid is prepared by mixing 0.787 g of sodium hydroxide and 26.69 g of water. The polymer solution, basic fluid, and air are spray dried through a tri-fluid nozzle with an inlet temperature of 160° C. at 0.5 bar air atomization pressure with flow rates of 7-9 mL/min at a 5:1 ratio of the acid to basic solution to produce a particle preparation (i.e., nutraceutical composition) comprising particles of 5-15 μm in diameter with a payload component loading of about 1-25%.

Without wishing to be bound by any particular theory, the present disclosure provides an insight that use of a basic fluid as described herein may provide in situ neutralization and may achieve production of water-insoluble particles, at least when utilized with particular polymer components (e.g., such as a polymer component that may be or comprise BMC) and/or with particular payload component (e.g., nutraceutical payload, such as a nutrient and/or probiotic payload as described herein).

### Example 17: Preparation of Particle Preparations Via Spray Drying with a Bi-Fluid Nozzle with a Single Aqueous Liquid Feed and Air Atomization

Preparing payload component: A DynoMill Multi-Lab bead mill is used to pre-micronize payload component with the following settings: (i) 2986 rpm, (ii) 250 mL/min pump/feed rate, and (iii) N2 purging over the beaker headspace. 500 mL deionized water is purged with N₂ for 30 minutes and is combined with: (i) 0.5 g Vit E TPGS, (ii) 50 g of nutraceutical (e.g., a carotenoid compound such as lutein, zeaxanthin, etc and/or a vitamin such as vitamin D, etc), and (iii) 400 mL of 0.65 mm beads. In some instances, the combination is milled for 60 min, adding additional deionized water as needed to maintain suitable viscosity.

Preparing particles comprising payload component: Acid, base, and/or surfactant is optionally used to dissolve the polymer component. A surfactant fluid is prepared by dissolving 0.1 g of surfactant (e.g., sodium dodecyl sulfate) into 150 g water at room temperature. A polymer solution is prepared by adding 3.68 g of 6N sulfuric acid (1.01 g sulfuric acid, 2.66 g water) to 10 g of Eudraguard Protect or other polymer component and stirring up to 1000 rpm for up to 2 hours until the polymer solution is clarified. Then, 1-10 g slurry is added to the polymer solution and is homogenized at 15000 rpm. The polymer solution and air are spray dried through a bi-fluid nozzle with an inlet temperature of 160° C. by pumping the feed solution at 5-6 ml/min with an air atomization pressure at 3 bar and to produce a particle preparation (i.e., nutraceutical composition) comprising particles of 5-15 μm in diameter with a payload component loading of about 1-25%.

### Example 18: Preparation of Particle Preparations Via Spray Drying with a Bi-Fluid Nozzle with a Single Organic Solvent Liquid Feed and Air Atomization

Preparing payload component: Optionally, the payload can be micronized prior to spray drying. A DynoMill Multi-Lab bead mill may be used to pre-micronize payload component with the following settings: (i) 2986 rpm, (ii) 250 mL/min pump/feed rate, and (iii) N2 purging over the beaker headspace. 500 mL deionized water is purged with N₂ for 30 minutes and is combined with: (i) 0.5 g Vit E TPGS, (ii) 50 g of nutraceutical (e.g., a carotenoid compound such as lutein, zeaxanthin, etc and/or a vitamin such as vitamin D, etc), and (iii) 400 mL of 0.65 mm beads. In some instances, the combination is milled for 60 min, adding additional deionized water as needed to maintain suitable viscosity.

Preparing particles comprising payload component: Solvent is used to dissolve the polymer component. A polymer solution is prepared by adding 140 ml of acetone to 9 g of Eudraguard Protect or other polymer component and stirring up to 1000 rpm for up to 2 hours until the polymer solution is clarified. Then, 1 g of payload component is added to the polymer solution and is homogenized at 15000 rpm. The polymer solution and air are spray dried through a bi-fluid nozzle with an inlet temperature of 160° C. by pumping the feed solution at 5-6 ml/min with an air atomization pressure at 3 bar and to produce a particle preparation (i.e., nutraceutical composition) comprising particles of 5-15 μm in size with a payload component loading of about 10%.

### Example 19: Preparation of Particle Preparations Via Emulsion

Preparing particles comprising payload component: Polymer solution is prepared by dissolving Eudraguard in DCM to a concentration of 100 mg/mL. Then, 2-30 mg of payload component is added to 1 mL of the polymer solution in a separate vial and mixed thoroughly. 20 mL of 10 mg/mL polyvinyl alcohol solution is added to a beaker and stirred at 300 rpm. The mixture of polymer solution and payload component is added into the stirring polyvinyl alcohol solution and is stirred further for 10 minutes. The resulting mixture of polymer solution, payload component, and polyvinyl alcohol solution is added to 100 mL of distilled or deionized water and is stirred at 500 rpm for 5 minutes. The resulting particles comprising payload component are allowed to settle without stirring. After settling, water is decanted off using a serological pipette. The particles comprising payload component is transferred to a vial to be lyophilized overnight to produce a particle preparation (i.e., nutraceutical composition) comprising particles 100-179 μm in size and with a payload component loading of 1-25%.

### Example 20: Summary of Particle Characteristics and Performance

TABLE 1
Certain technical parameters of non-limiting exemplary particle
preparations (i.e., nutraceutical compositions).

| | |
|---|---|
| Feature: Conditions | Results: |
| | All ‘x’ increases are compared to “free” compound (i.e., not |
| | incorporated into or combined with a particle preparation as |
| | described herein) |
| Particle sizes: 5-3000 μm | Particle preparations size can be controlled between 5-3000 μm |
| Particle loadings: 2-25.6% for | Lutein particle preparations: 2, 6.3, 20.1, 25.6% (w/w %) |
| lutein; 2-15% for zeaxanthin, | Zeaxanthin particle preparations: 2.1, 8.2, 12.1, 15% (w/w %) |
| 0.9-10.7% for vitamin D | Vitamin D particle preparations: 0.9, 2.1, 3.6, 4.5, 10.7% |
| Light protection: | Lutein particle preparations: |
| 24, 48, 72 hours, >83000 lux | >39x protection (72 hours) |
| | >1.8x protection (48 hours) |
| | Zeaxanthin particle preparations: |
| | >8x protection (72 hours) |
| | >3.4x protection (48 hours) |
| | Vitamin D particle preparations: |
| | >28x protection (24 hours) |
| | >9.9x protection (48 hours) |
| Water stability: 200 days, | Lutein particle preparations: >650x protection |
| ambient temperature, in the dark | Zeaxanthin particle preparations: >3.8x protection |
| Milk stability: Skim milk, 4° C., | Lutein particle preparations: >55% stable |
| 2 weeks | Zeaxanthin particle preparations: >56% stable |
| Release: 2 hours | Lutein, zeaxanthin, vitamin D particle preparations: |
| Simulated gastric fluid | >95% release in simulated gastric fluid (SGF) at 15 minutes |
| Room Temp or Boiling Water | (stomach conditions) |
| | <5% release in boiling or ambient temperature water at 2 hours |
| Water activity | Lutein, zeaxanthin, vitamin D particle preparations: <0.2000 |
| | Competitor OmniActive Lutemax 2020 ® Beadlets: >0.3000 |
| Morphology | Spherical, donut-shaped, cylindrical, circular, disc-shaped, |
| | worm-like, irregular-shaped particle preparations, etc.) |
| Mixing with probiotics | MPs mix homogenously with Lacticaseibacillus rhamnosus |
| 185 mg probiotic | powder; Competitor product (e.g., OmniActive Lutemax |
| Lacticaseibacillus rhamnosus | 2020 ®) mixes heterogeneously |
| 110 mg lutein MPs | |
| 20 mg zeaxanthin MPs | |

### Example 21: Quantification of Lutein and/or Zeaxanthin in Exemplary Preparations

This example demonstrates exemplary protocols for quantifying disclosed preparations comprising lutein and/or zeaxanthin (e.g., microparticle preparations). For example, exemplary protocols may include analytical techniques, such as high-performance liquid chromatography (HPLC), to quantitatively determine amounts (e.g., percent loading) of lutein and/or zeaxanthin in disclosed preparations.

Exemplary protocols for quantifying lutein and/or zeaxanthin content in exemplary preparations are presented in Appendices A-L, the entire contents of each of which is incorporated herein in its entirety.

In some instances, lutein and/or zeaxanthin components are extracted from exemplary preparations before quantifying lutein and/or zeaxanthin content in disclosed preparations, exemplary protocols of which are disclosed herein.

In some instances, HPLC analysis is performed utilizing a high-performance liquid chromatograph (HPLC system) with a UV/VIS detector. For example, an exemplary HPLC system may be an Agilent 1260, Agilent 1290, or other equivalent systems comprising a suitable carotenoid column (e.g., an YMC C30 carotenoid column (e.g., 5 um, 250×4.6 mm)). Further, an exemplary HPLC system may comprise an Agilent MWD detector or DAD detector having a detection wavelength of 450 nm and a slit width of 4 nm.

HPLC Analysis Materials: Table 2 presents exemplary materials (e.g., analytes) that are utilized in disclosed HPLC analysis protocols. Presented materials are equilibrated to room temperature for at least 30 minutes before preparing for analysis.

TABLE 2
Exemplary HPLC Analysis Materials

| Material | Manufacturer | Purity | Notes |
|---|---|---|---|
| Free Lutein | CHENGUANG | About 75% lutein | To be used no |
| (powder)* | BIOTECH GROUP | | more than 2 |
| | CO., LTD, or IOSA | | years from the |
| | | | date of |
| | | | manufacturing |
| Free Zeaxanthin | Shandong Tianyin | About 70% Zeaxanthin | To be used no |
| (powder)* | Biotechnology Co., | | more than 2 |
| | LTD, or IOSA | | years from the |
| | | | date of |
| | | | manufacturing |
| Lutein and/or | Prepared as | Lutein: X % loading | N/A |
| Zeaxanthin | disclosed herein | Zeaxanthin: X % loading | |
| Preparations (e.g., | | (e.g., as disclosed herein | |
| not included in milk | | including Appendices) | |
| powder, milk, or | | | |
| capsules) | | | |

Tables 3-5 present exemplary solutions that are utilized in disclosed protocols. For example, solutions may be primary stock solutions (PS), calibration standards (i.e., spiking solutions) for lutein, calibration standards (i.e., spiking solutions) for zeaxanthin, or quality control samples (i.e., samples) comprising disclosed lutein and zeaxanthin particle preparations.

TABLE 3
Exemplary concentrations for analysis solutions which include
primary stock solutions, calibration standards/spiking solutions,
quality control samples of exemplary preparations.

| Solution | Concentrations (range) |
|---|---|
| Primary Stock | Free Lutein (75% purity) at 1.333 mg/mL or |
| Solutions (PS) | higher in acetone (freshly prepared, vortexed, |
| | equivalent to 1.0 mg/mL pure lutein) |
| | Free Zeaxanthin (70% purity) at 1.429 mg/mL |
| | or higher in acetone (freshly prepared, vortexed, |
| | equivalent to 1.0 mg/mL pure zeaxanthin) |
| | Lutein and Zeaxanthin preparation at 1 mg/mL in |
| | acetone (freshly prepared, vortexed) |
| Internal Standard | N/A |
| Solution (IS) | |
| Calibration Standard | 100 μg/mL Lutein in acetone |
| Solutions/Spiking | 50 μg/mL Lutein in acetone |
| Solutions for | 25 μg/mL Lutein in acetone |
| Lutein (diluted from | 12.5 μg/mL Lutein in acetone |
| Lutein PS solutions) | 6.25 μg/mL Lutein in acetone |
| | 3.125 μg/mL Lutein in acetone |
| | 1.563 μg/mL Lutein in acetone |
| | 0.781 μg/mL Lutein in acetone |
| | 0.391 ug/mL Lutein in acetone |
| Calibration Standard | 100 μg/mL Zeaxanthin in acetone |
| Solutions/Spiking | 50 μg/mL Zeaxanthin in acetone |
| Solutions for | 25 μg/mL Zeaxanthin in acetone |
| Zeaxanthin (diluted | 12.5 μg/mL Zeaxanthin in acetone |
| from Zeaxanthin PS | 6.25 μg/mL Zeaxanthin in acetone |
| solutions) | 3.125 μg/mL Zeaxanthin in acetone |
| | 1.563 μg/mL Zeaxanthin in acetone |
| | 0.781 μg/mL Zeaxanthin in acetone |
| | 0.391 μg/mL Zeaxanthin in acetone |
| Quality Control | As necessary to make desired performance |
| Solutions for Lutein | characteristics. |
| and/or Zeaxanthin | |
| Preparations, diluted | |
| from PS solutions | |

TABLE 4
Exemplary concentrations of lutein calibration standards/spiking solutions.

| | | Vol. | Vol. | | | |
|---|---|---|---|---|---|---|
| | Spiking | Spiking | acetone | | | |
| | Solution | solution | (or other | Total | | |
| Spiking | Conc. | to add | solvent) | Volume | Conc. | |
| Solution ID | (μg/mL) | (μL) | (μL) | (μL) b | (μg/mL) | STD/QC ID |
| Lutein stock | 1000* | 200 | 1800 | 1000 | 100 | L STD 9 |
| | (1333)** | | | | | |
| L STD 9 | 100 | 1000 | 1000 | | 50 | L STD 8 |
| L STD 8 | 50 | 1000 | 1000 | | 25 | L STD 7 |
| L STD 7 | 25 | 1000 | 1000 | | 12.5 | L STD 6 |
| L STD 6 | 12.5 | 1000 | 1000 | | 6.25 | L STD 5 |
| L STD 5 | 6.25 | 1000 | 1000 | | 3.125 | L STD 4 |
| L STD 4 | 3.125 | 1000 | 1000 | | 1.563 | L STD 3 |
| L STD 3 | 1.563 | 1000 | 1000 | | 0.781 | L STD 2 |
| L STD 2 | 0.781 | 1000 | 1000 | | 0.391 | L STD 1 |
| | | | 2000 | | 0 | Blank |

*Calculated based on pure Free Lutein;
**Calculated based on 75% pure Free Lutein.

TABLE 5
Exemplary concentrations of zeaxanthin calibration standards/spiking solutions.

| | | Vol. | Vol. | | | |
|---|---|---|---|---|---|---|
| | Spiking | Spiking | Acetone | | | |
| | Solution | solution | (or other | Total | | |
| | Conc. | to add | solvent) | Volume | Conc. | STD/QC |
| Spiking Solution ID | (μg/mL) | (μL) | (μL) | (μL) ^{b} | (μg/mL) | ID |
| Zeaxanthin stock | 1000 | 200 | 1800 | 1000 | 100 | Z STD 9 |
| | (1429) * | | | | | |
| Z STD 9 | 100 | 1000 | 1000 | | 50 | Z STD 8 |
| Z STD 8 | 50 | 1000 | 1000 | | 25 | Z STD 7 |
| Z STD 7 | 25 | 1000 | 1000 | | 12.5 | Z STD 6 |
| Z STD 6 | 12.5 | 1000 | 1000 | | 6.25 | Z STD 5 |
| Z STD 5 | 6.25 | 1000 | 1000 | | 3.125 | Z STD 4 |
| Z STD 4 | 3.125 | 1000 | 1000 | | 1.563 | Z STD 3 |
| Z STD 3 | 1.563 | 1000 | 1000 | | 0.781 | Z STD 2 |
| Z STD 2 | 0.781 | 1000 | 1000 | | 0.391 | Z STD 1 |
| | | | 2000 | | 0 | Blank |

*Calculated based on pure Free Zeaxanthin;
**Calculated based on 70% pure Free Zeaxanthin.

Tables 6-8 present exemplary solutions that are utilized in disclosed protocols, for examples in protocols that require extracting lutein and/or zeaxanthin from disclosed preparations. For example, solutions may be primary stock solutions (PS), calibration standards (i.e., spiking solutions) for lutein, calibration standards (i.e., spiking solutions) for zeaxanthin, or quality control samples (i.e., samples) comprising disclosed lutein and zeaxanthin particle preparations. In some instances, lutein and zeaxanthin particle preparations may be included in powdered milk, milk, or capsules.

TABLE 6
Exemplary concentrations

| Solutions | Concentration (range) |
|---|---|
| Primary Stock Solutions (PS) | Lutein at 1.333 mg/mL or higher in |
| | pure milk, powdered milk, or water |
| | (freshly prepared, vortexed, |
| | equivalent to 1.0 mg/mL pure lutein) |
| | Zeaxanthin at 1.429 mg/mL or higher |
| | in pure milk, powdered milk, or water |
| | (freshly prepared, vortexed, equivalent |
| | to 1.0 mg/mL pure zeaxanthin) |
| | Serial dilution to prepare for the |
| | calibration standards is performed |
| | after adding EtOH (sample:EtOH 1:1) |
| | and diluted into EtOH:sample (1:1) |
| | mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) | 1.563 μg/mL Lutein |
| | 0.781 μg/mL Lutein |
| | 0.391 ug/mL Lutein |
| | 0.195 ug/mL Lutein |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.25 μg/mL Zeaxanthin |
| during extraction process, not | 3.125 μg/mL Zeaxanthin |
| actual concentration) | 1.563 μg/mL Zeaxanthin |
| | 0.781 μg/mL Zeaxanthin |
| | 0.391 μg/mL Zeaxanthin |
| | 0.195 ug/mL Zeaxanthin |
| Quality Control Samples for | N/A, to be included in the future |
| Lutein and/or Zeaxanthin, | |
| diluted from PS solutions | |

TABLE 7
Exemplary concentrations of lutein calibration standards/spiking solutions.

| | | Vol. | | | | | | | |
|---|---|---|---|---|---|---|---|---|---|
| | Spiking | Spiking | Vol. | | | Vol. | | | |
| Spiking | Solution | solution | EtOH: powder | Total | | DCM | | | Nominal |
| solution | Conc. | to add | milk mixture | Volume | Stock | to add | Dilution | STD | Std conc. |
| ID | (μg/mL) | (μL) | (μL) | (μL) ^{b} | ID | (uL) | in Acetone | ID | (μg/mL)** |
| Lutein | 1000 | | | 1000 | L stock 10 | 500 | 1/10x | L STD 10 | 100 |
| Primary | (1333)* | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Calculated based on pure Free Lutein;
**Calculated based on 75% pure Free Lutein.
Concentrations are nominal concentrations, as similarly described in Table 6.

TABLE 8
Exemplary concentrations of zeaxanthin calibration standards/spiking solutions.

| | | Vol. | | | | | | | |
|---|---|---|---|---|---|---|---|---|---|
| | Spiking | Spiking | Vol. | | | Vol. | | | |
| Spiking | Solution | solution | EtOH: powder | Total | | DCM | Dilution | | Nominal |
| solution | Conc. | to add | milk mixture | Volume | Stock | to add | in | STD | Std conc. |
| ID | (μg/mL) | (μL) | (μL) | (μL) ^{b} | ID | (uL) | Acetone | ID | (μg/mL)** |
| Zeaxanthin | 1000 | | | 1000 | Z stock 10 | 500 | 1/10x | Z STD 10 | 100 |
| Primary | (1429)* | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Calculated based on pure Free Zeaxanthin;
**Calculated based on 70% pure Free Zeaxanthin.
Concentrations are nominal concentrations, as similarly described in Table 6.

In some instances, primary stock solutions are freshly prepared due to potential instability of analysis materials.

In some instances, concentrations of analysis materials in primary stock solutions are varied depending on a utilized HPLC system sensitivity and maximum detection range.

In some instances, HPLC analysis materials are dissolved in an optimal solvent (i.e., a diluent), such as acetone.

In some instances, alternative solvent compositions can be used for preparing HPLC analysis solutions, including but not limited to, Dichloromethane: Acetone mixture (1:9, 2:8, 3:7, 4:6, 5:5 v/v), Methyl tert-butyl ether (MTBE): Acetone mixture (1:9, 2:8, 3:7, 4:6, 5:5 v/v), etc. In some instances, solvent compositions are of suitable purity, e.g., >95% purity. For example, solvent compositions may be referred to as “reagent grade”, “HPLC grade”, or of another title known to be sufficiently pure for analysis purposes.

In some instances, calibration standards and quality control samples must be treated with the same solvents.

Exemplary HPLC Mobile Phases, Diluent Blanks: An exemplary HPLC analysis may be performed utilizing the following mobile phases and diluent blanks.

Mobile Phase A: HPLC analysis requires the preparation of a first mobile phase (e.g., an aqueous phase) comprising (i) an exemplary amount of HPLC grade water, (ii) an exemplary amount of a solution prepared by thoroughly mixing 1 mL of formic acid (e.g., reagent grade formic acid) in 1000 mL of HPLC grade water, or (iii) an exemplary amount of a suitable pre-made solution of water with 0.1% formic acid.

Mobile Phase B: HPLC analysis requires the preparation of a second mobile phase (e.g., an organic phase) comprising HPLC grade acetone.

Diluent Blank: HPLC analysis requires the preparation of a diluent blank comprising HPLC grade acetone.

Exemplary HPLC Conditions and Parameters: An exemplary HPLC analysis is performed using the conditions presented in Tables 9-10.

TABLE 9
Exemplary conditions and parameters for running HPLC

| Column Temperature | 20° | C. |
|---|---|---|
| Autosampler Temperature | 2-8° | C. |
| Data System | Agilent CDS | |
| Mobile Phase A | HPLC grade water (or 0.1% | |
| | formic acid in water) | |
| Mobile Phase B | HPLC grade acetone | |
| Gradient | N/A (Isocratic Mode) | |
| Mobile phase flow rate | 1.0 | mL/min |
| Injection Volume | 20 | μL |
| Run Time | 5 | minutes |

| | |
|---|---|
| Expected Analyte Retention | Lutein: 4 minutes |
| Times | Zeaxanthin: 4.5 minutes |
| Needle Washing Solvent: | May be acetone or 1:1 (v/v) |
| | water:acetonitrile |

TABLE 10
Exemplary mobile phase compositions

| Step | Time (min) | Mobile Phase A (%) | Mobile Phase B (%) |
|---|---|---|---|
| 0 | 0 | 1 | 99* |
| 1 | 5 | 1 | 99* |

Exemplary Mobile Phase A: B ratios can be varied depending on needs, including but not limited to, 2:98, 3:97, 4:96, 5:95, 6:94, 7:93, 8:92, 9:91, 10:90, 11:89, etc. Without being bound by any theory, increased proportions of Mobile Phase A may cause increased retention times for lutein and zeaxanthin (e.g., retention time peaks characteristic of lutein and/or zeaxanthin are shifted toward higher retention times), which may cause for longer HPLC run times. Additionally or alternatively, utilizing increased proportions of Mobile Phase A may provide for greater separation of peaks in retention time between lutein and zeaxanthin.

Exemplary HPLC Analysis Sequences: Prior to performing HPLC analysis sequences, such as that presented in Table 8, an exemplary HPLC system and column are equilibrated for at least 15 minute before performing an analysis (i.e., before injecting samples.) Utilizing an equilibrated HPLC system and column, HPLC analysis proceeds by injections in a manner exemplified in Table 8.

TABLE 8
Exemplary injection sequence for HPLC analysis

| Sample Description | Injections |
|---|---|
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |
| Lutein Calibration Std 1 to Std 9 | 1 |
| Diluent Blank | 1 |
| Zeaxanthin Calibration Std 1 to Std 9 | 1 |
| Diluent Blank | 1 |
| Unknown Sample (Lutein/Zeaxanthin/MPs) | 1 |
| Diluent Blank | 1 |
| Lutein Calibration Std 1 to Std 9 | 1 |
| Diluent Blank | 1 |
| Zeaxanthin Calibration Std 1 to Std 9 | 1 |
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |

In some instances, lutein system suitability injections are not performed. In some instances, zeaxanthin system suitability injections are not performed. In some instances, lutein calibration injections are not performed. In some instances, zeaxanthin calibration injections are not performed.

HPLC Data Analysis: HPLC system suitability is determined by comparing a chromatographic profile of 100 μg/mL lutein standards and zeaxanthin standards. For example, standards are compared to “very fresh” lutein standards and zeaxanthin standards. In some instances, an HPLC system is suitable when % RSD≤3 for the main peak areas of the six 100 g/mL lutein and zeaxanthin standard injections. Additionally or alternatively, other lutein standard and zeaxanthin standard concentrations may be utilized for quality control purposes.

Calibration standard curves may be determined by using the average peak area from the two injections of each standard level, generating a standard curve and determining the coefficient of determination (R²), slope, and y-intercept.

For each lutein and zeaxanthin standard, at least 75% of standards are included in calculation of the calibration standard curves (e.g. 9 levels×2 set=18 Stds, maximum 4 out of 18 are allowed to be excluded as outliers).

In some instances, at LLOQ (lowest limit of quantification), a signal/background ratio must be higher than 5.

In some instances, a standard curve has an R²>0.99.

In some instances, percent recovery of standard levels must be within 90% to 110% of its label claim.

Calculating lutein and/or zeaxanthin concentrations in samples: Using the standard curves determined above for lutein and zeaxanthin, lutein and zeaxanthin concentrations are calculated using the following equation:

$$L or Z ( \frac{mg}{mL} ) = \frac{{A}_{Test} - y intercept}{slop e} \times DF \times 0.001$$

In some instances, Aₜₑₛₜ=Average main peak area of either Lutein or Zeaxanthin.

In some instances, DF=dilution factor of the test sample.

In some instances, 0.001=convert μg/mL to mg/mL.

Percent recovery of a sample is calculated by utilizing the following equation:

$$\frac{Sample L or Z concentration ( \frac{mg}{mL} )}{Label Claim of L or Z test sample ( \frac{mg}{mL} )} \times 100$$

In some instances, percent recovery of a sample must be within 85% to 110% of its label claim.

Percent amount of lutein and/or zeaxanthin loading is calculated by the following equation:

$$\frac{Sample L or Z concentration ( \frac{mg}{mL} )}{MP concentration ( \frac{mg}{mL} )} \times 100$$

In some instances, a percent amount of lutein and/or zeaxanthin loaded in a test sample is reported to one decimal place (e.g. 96.5%).

In some instances, a percent amount of free lutein and/or zeaxanthin in a test sample is reported to one decimal place (e.g. 3.5%).

### Example 22: Extraction of Lutein and/or Zeaxanthin from Exemplary Preparations

This example demonstrates exemplary protocols for the extraction of lutein, zeaxanthin, or both from exemplary preparations. Extracting these components from preparations may be necessary for quantifying lutein (L) and/or zeaxanthin (Z) content within disclosed preparations.

For example, extraction of lutein and/or zeaxanthin from exemplary preparations occurs utilizing liquid-liquid extraction methods. For example, liquid-liquid extraction methods may utilize solvents such as ethanol and/or dichloromethane (DCM).

Extraction of lutein and/or zeaxanthin from powdered milk preparations: Exemplary protocols for extracting lutein and/or zeaxanthin from powdered milk preparations is presented in Appendices B, F, and J, the contents of each of which is incorporated herein in its entirety.

Extraction of lutein and/or zeaxanthin from milk preparations: Exemplary protocols for extracting lutein and/or zeaxanthin from milk preparations is presented in Appendices C, G, and K, the contents of each of which is incorporated herein in its entirety.

Extraction of lutein and/or zeaxanthin from capsule preparations: Exemplary protocols for extracting lutein and/or zeaxanthin from capsules is presented in Appendices D, H, and L, the contents of each of which is incorporated herein in its entirety. Example 23: Microparticle Stability and Dispersibility

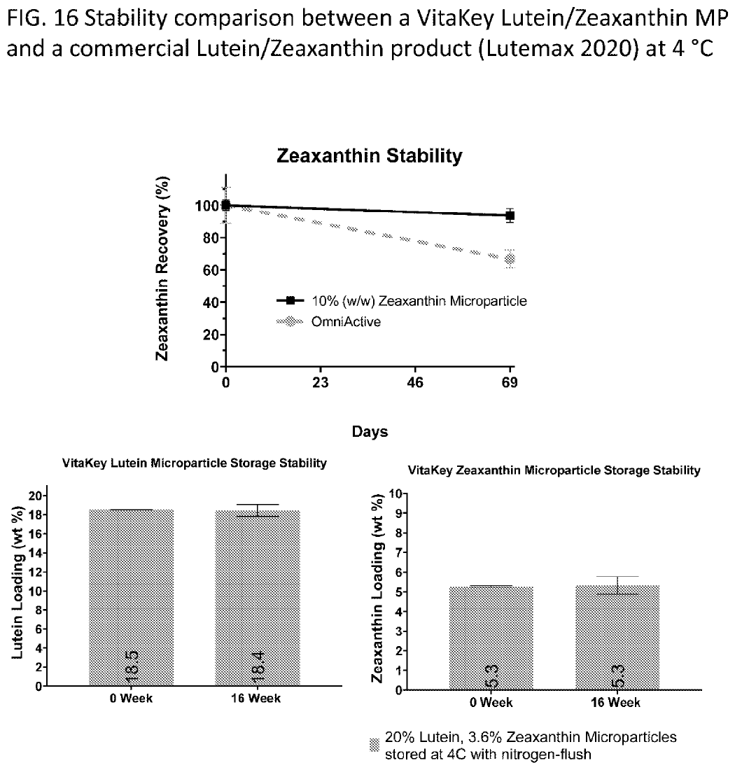

FIG. 16 describes zeaxanthin stability in a zeaxanthin microparticle (10% w/w zeaxanthin) as compared to a commercial product (OmniActive Lutemax 2020 sealed conditions stored at 4 C (refrigerated). In this figure, zeaxanthin microparticle at 4 C outperforms OmniActive lutemax 2020. In FIGS. 16B and 16C, a 20% w/w lutein-3.6% w/w zeaxanthin microparticle maintains stability after 16 weeks of storage in nitrogen-flushed conditions at 4 C. This indicates that both lutein and zeaxanthin microparticles are stable when co-formulated in a single microparticle.

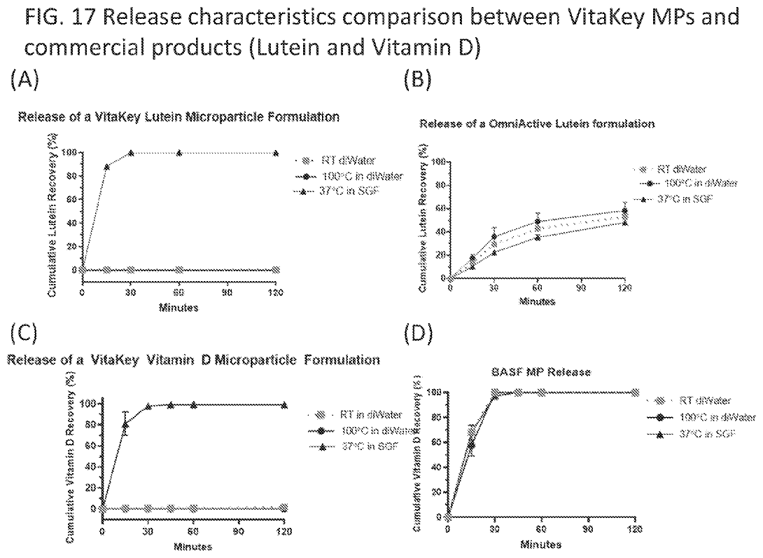

FIG. 17 describes controlled release of lutein and vitamin D from pH-responsive microparticles, as compared to commercial microparticle products. Lutein and vitamin D microparticles release ˜0% payload when in neutral water or boiling water over a 2 hour period; in contrast, lutein and vitamin D microparticles release 100% payload in simulated gastric fluid at 37 C. For the commercial products, both OmniActive Lutemax 2020 and BASF vitamin D release >40% and ˜100% of payloads in water at both room temperature and boiling and in 37 C simulated gastric fluid. Taken together, this data demonstrates that commercial products cannot provide pH-responsive release and thus do not maintain payload encapsulation in aqueous environments, unlike the pH-responsive formulations described here.

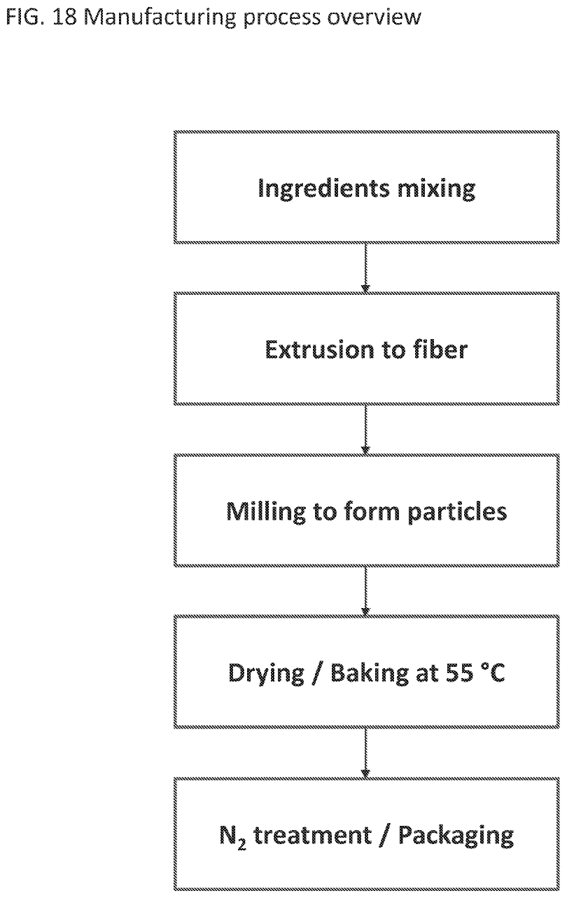

FIG. 18 shows, in a non-limiting example, a schematic of an extrusion method used to manufacture lutein and zeaxanthin, or vitamin D, microparticles of low solvent and low water activity.

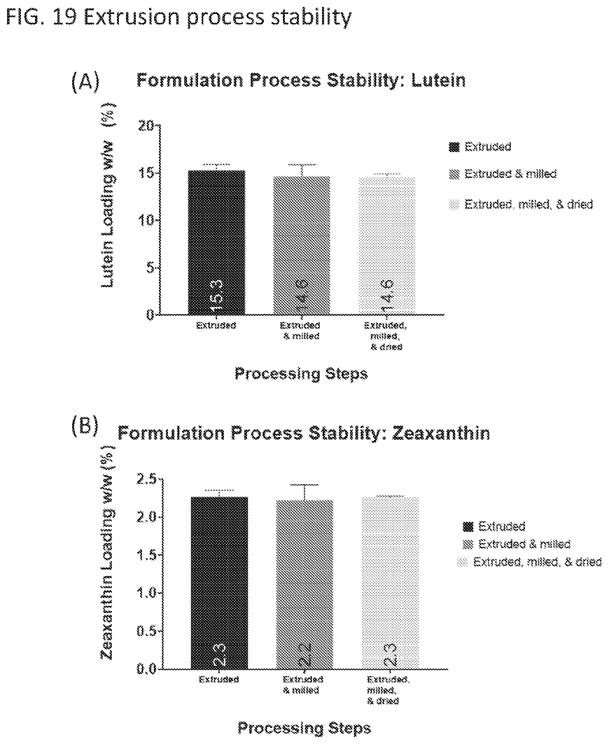

FIG. 19 describes stability of lutein and zeaxanthin microparticles during the extrusion-based manufacturing process. This data demonstrates that during manufacturing (extrusion to milling to drying to bagging), lutein and zeaxanthin remain stable throughout the whole process. As such, we have developed robust manufacturing processes that do not degrade or damage the payloads.

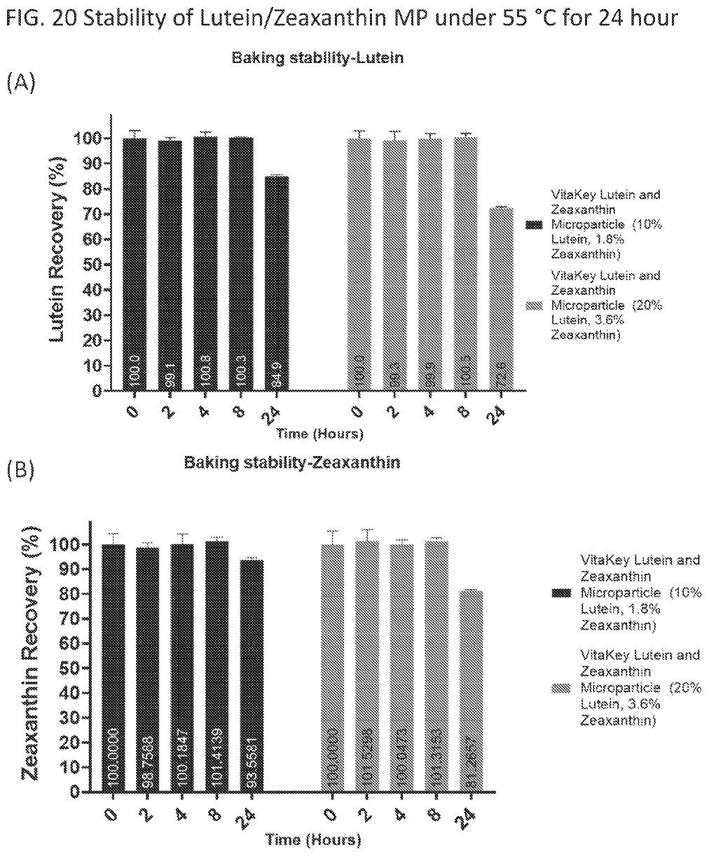

FIG. 20 describes stability of lutein and zeaxanthin microparticles during baking at 55 C for up to 24 hours. This data demonstrates that for up to 8 hours, minimal losses in payload stability are observed during baking at 55 C. At 24 hours, up to 28% losses (and as little as 7% losses) in payload stability are observed in various lutein and zeaxanthin formulations. Altogether, lutein and zeaxanthin microparticles are stable during baking in oxygen-rich conditions (exposed to atmospheric air).

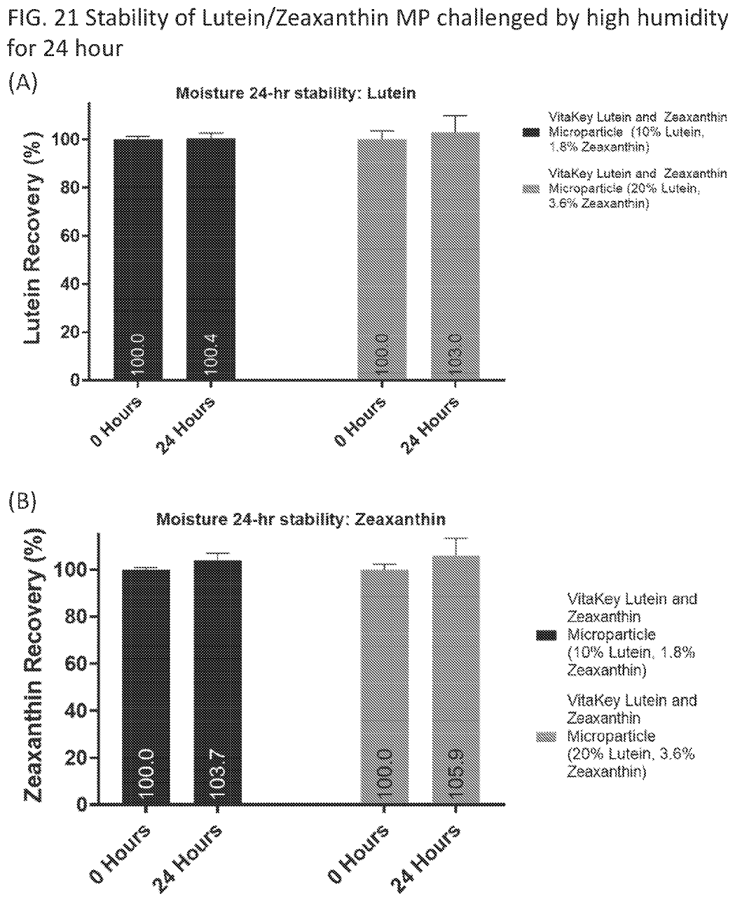

FIG. 21 describes stability of lutein and zeaxanthin microparticles during exposure to 75% relative humidity for 24 hours. This data demonstrates that for up to 24 hours, minimal losses in payload stability are observed at room temperature at 75% relative humidity. Altogether, lutein and zeaxanthin microparticles are stable during exposure to high moisture in oxygen-rich conditions (exposed to atmospheric air).

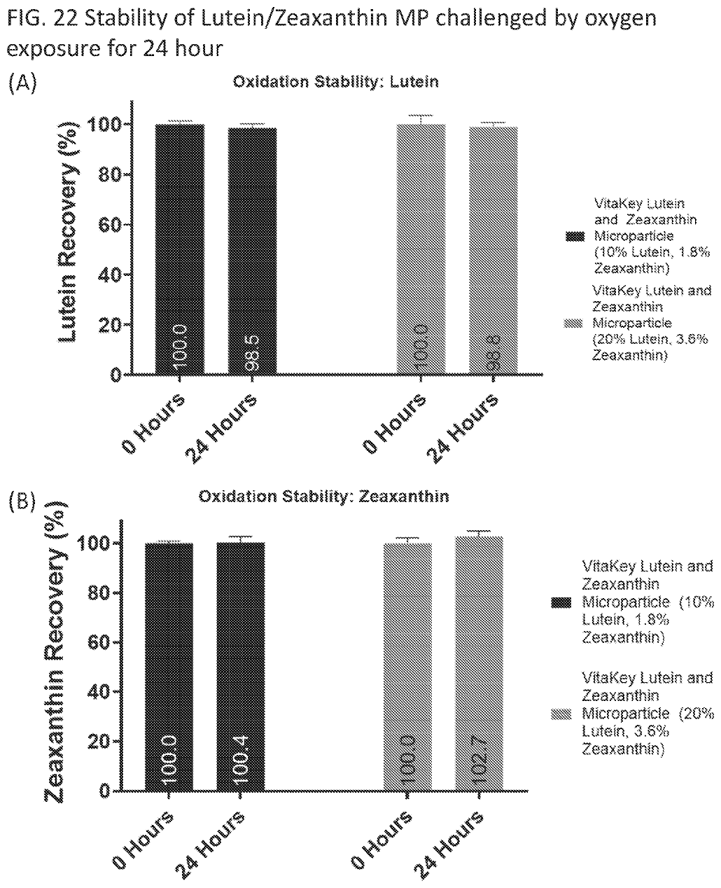

FIG. 22 describes stability of lutein and zeaxanthin microparticles during exposure to oxygen (air atmosphere) for 24 hours. This data demonstrates that for up to 24 hours, minimal losses in payload stability are observed at room temperature when exposed to normal atmosphere (1 atm). Altogether, lutein and zeaxanthin microparticles are stable during exposure to oxygen-rich conditions (exposed to atmospheric air).

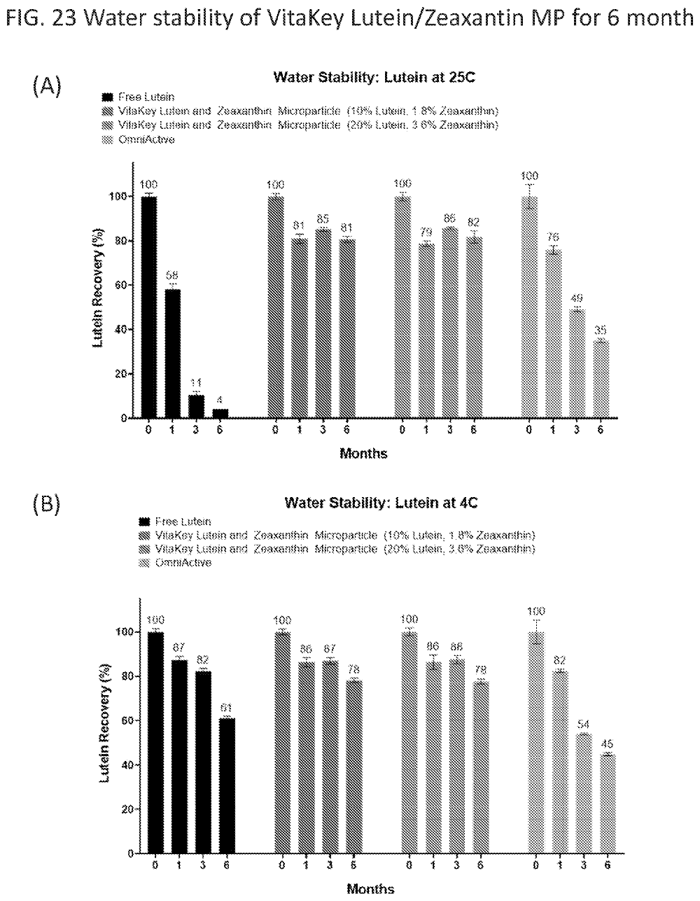

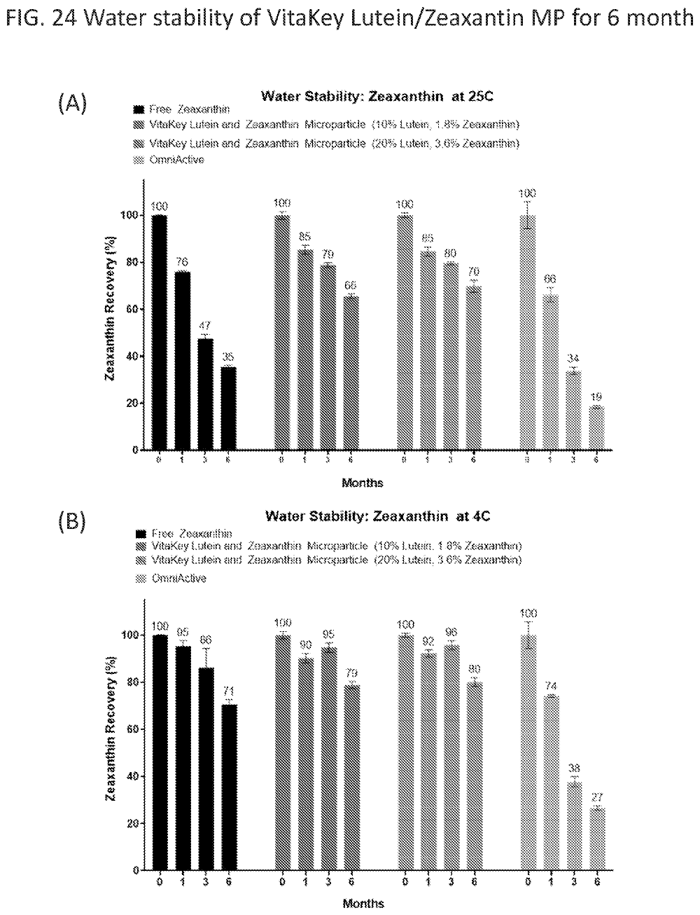

FIG. 23 and FIG. 24 describes stability of lutein and zeaxanthin microparticles during exposure to water for up to 6 months, compared to non-encapsulated lutein and zeaxanthin and a commercial product (OmniActive Lutemax 2020). Over a 6 month period, microparticles demonstrate the ability to increase the chemical stability of both lutein and zeaxanthin as compared to non-encapsulated free lutein/zeaxanthin and OmniActive Lutemax 2020. Two different loadings of microparticles were investigated and demonstrated to be provide enhanced stability in water, by over 3-fold compared to commercial products in some cases and by over 40-fold compared to free payloads in other cases.

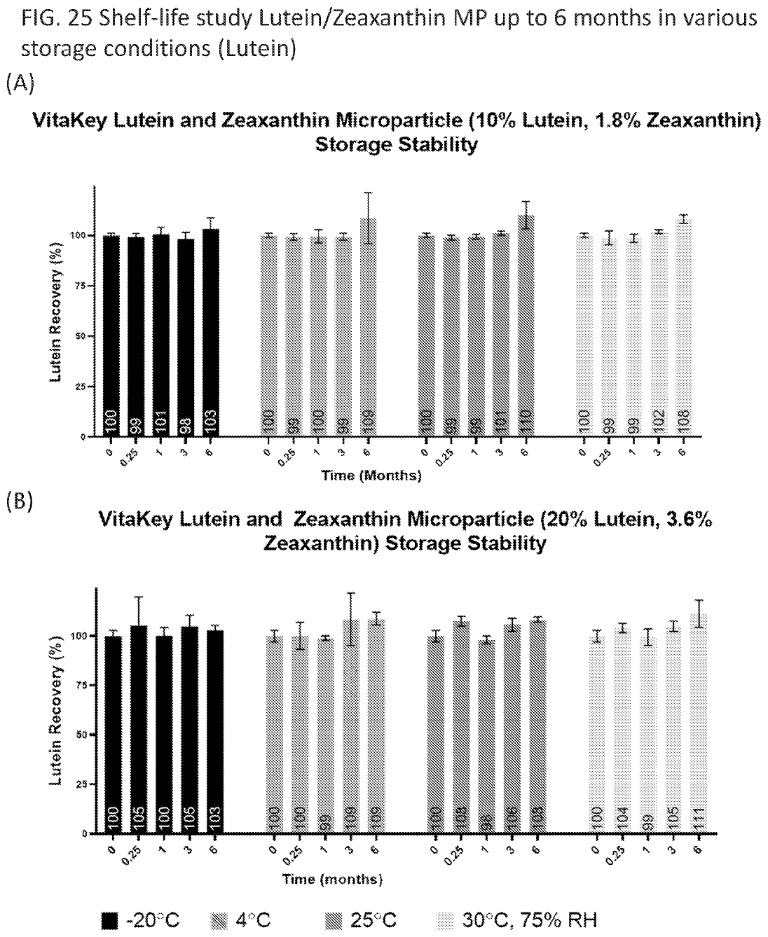

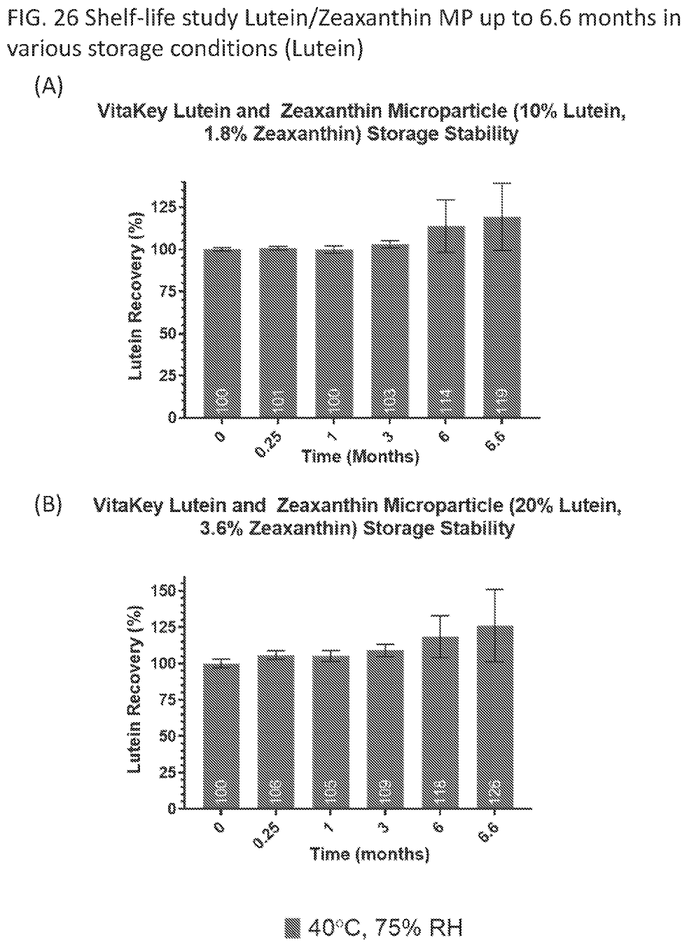

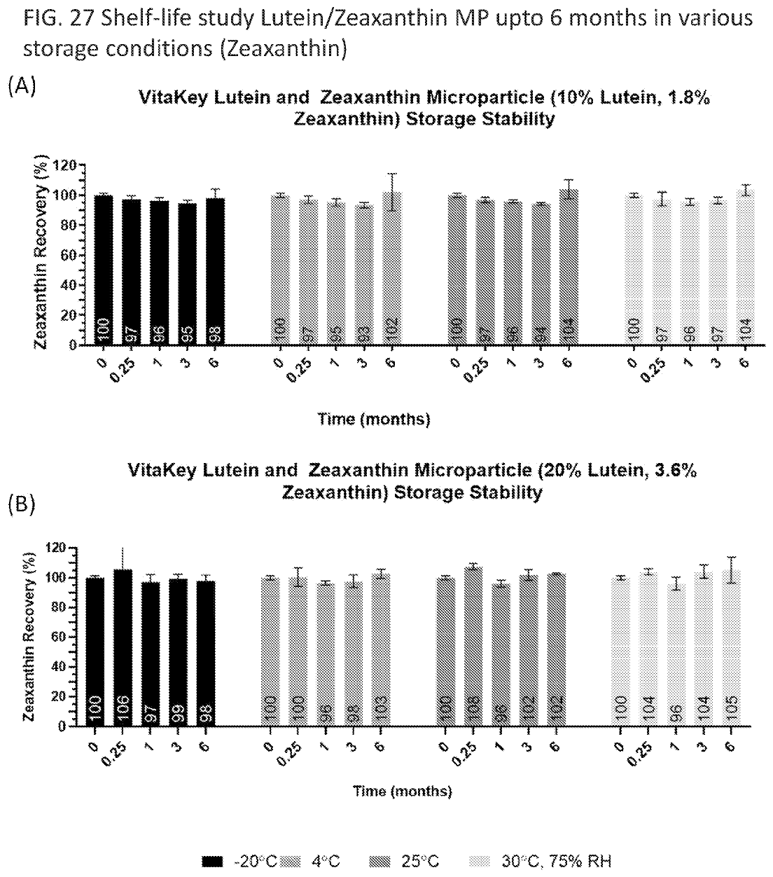

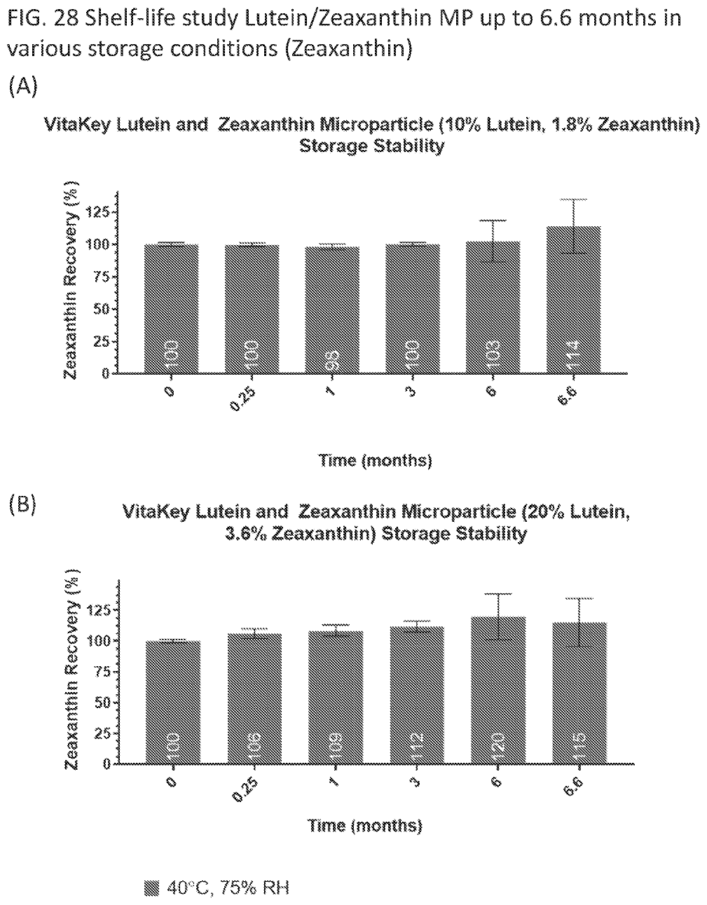

FIGS. 25-28 describes stability of lutein and zeaxanthin microparticles during storage at −20 C, 4 C, 25 C, and 30 C at 75% relative humidity for up to 6 months; and at 40 C at 75% relative humidity for up to 6.6 months. This data demonstrates that for up to 6 and 6.6 months, minimal losses in payload stability are observed under the studied storage conditions. Altogether, lutein and zeaxanthin microparticles are stable during exposure to oxygen-rich conditions (exposed to atmospheric air).

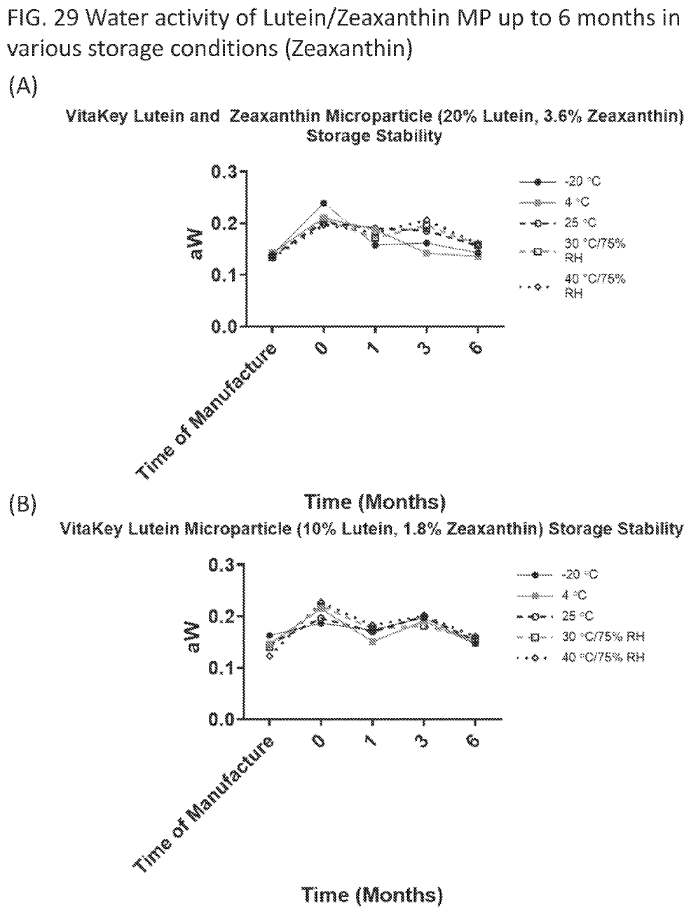

FIG. 29 describes water activity of lutein and zeaxanthin microparticles during storage at −20 C, 4 C, 25 C, 30 C at 75% relative humidity; and at 40 C at 75% relative humidity for up to 6 months. This data demonstrates that water activity of the microparticle formulations can be kept low (below 0.30 and below 0.20 at 6 months). Altogether, lutein and zeaxanthin microparticles maintain their low water activity during storage.

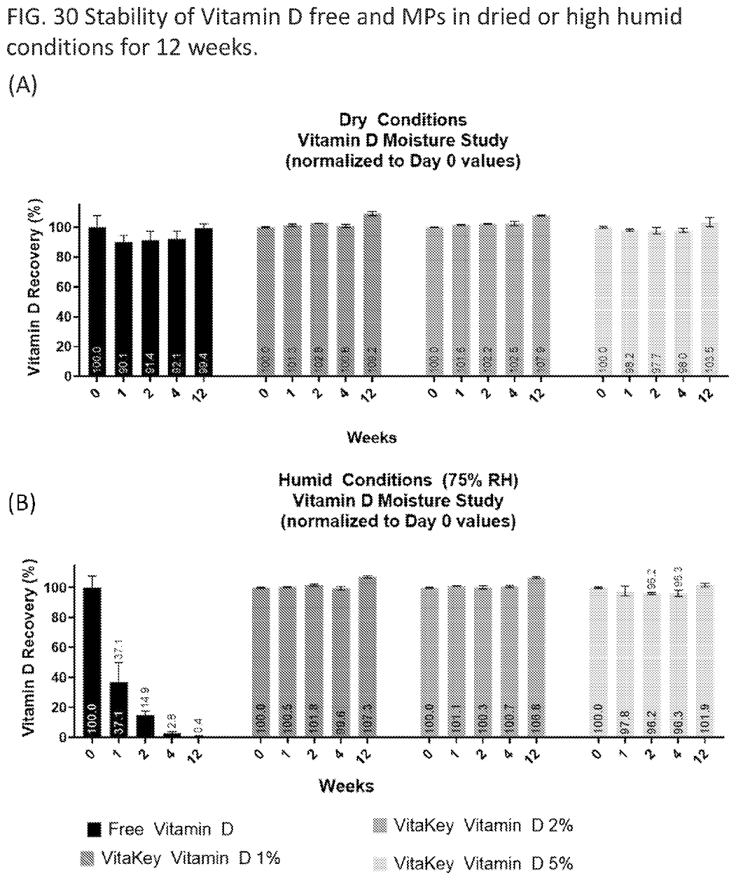

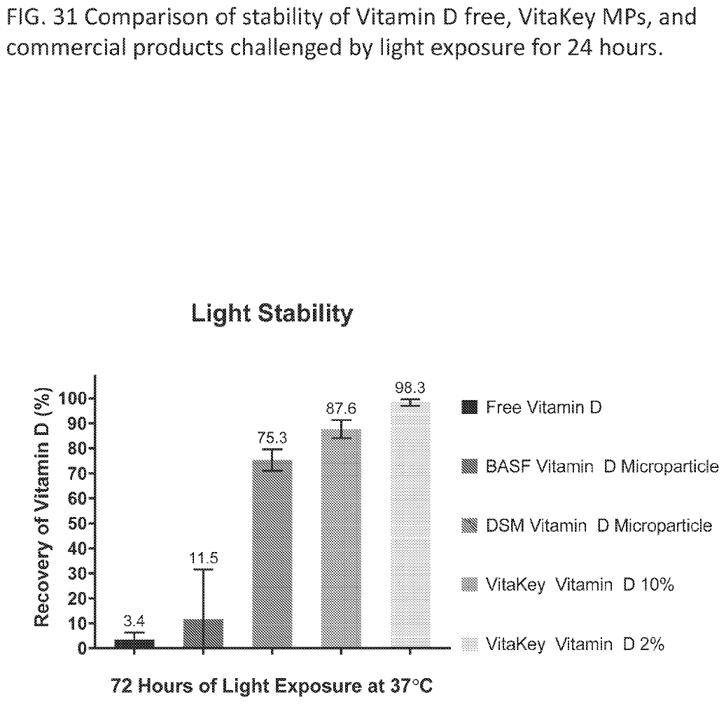

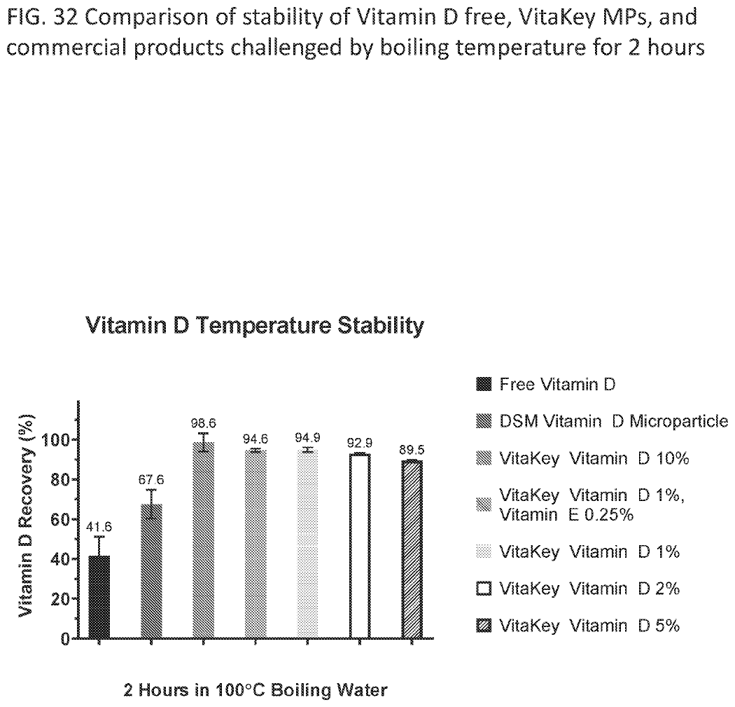

FIGS. 30-32 describe increased stability of microparticle vitamin D as compared to free vitamin D and various commercial vitamin D microparticles (DSM, BASF). Microparticle vitamin D demonstrates high stability, over 250-fold more stable than free vitamin D, when exposed to humid (75% relative humidity conditions) up to 12 weeks. Microparticle vitamin D demonstrates high stability, over 28-fold more stable than free vitamin D and over 11.5-fold more stable than BASF vitamin D and over 1.3-fold more stable than DSM vitamin D when exposed to 85,000 lux over a 72 hour period at 37 C. Microparticle vitamin D demonstrates high stability, over 2.3-fold more stable than free vitamin D and over 1.4-fold more stable than DSM vitamin D 2 hour period at 100 C in boiling water. Altogether, Microparticle vitamin D demonstrates high stability over other commercial products and free vitamin D in various challenging conditions.

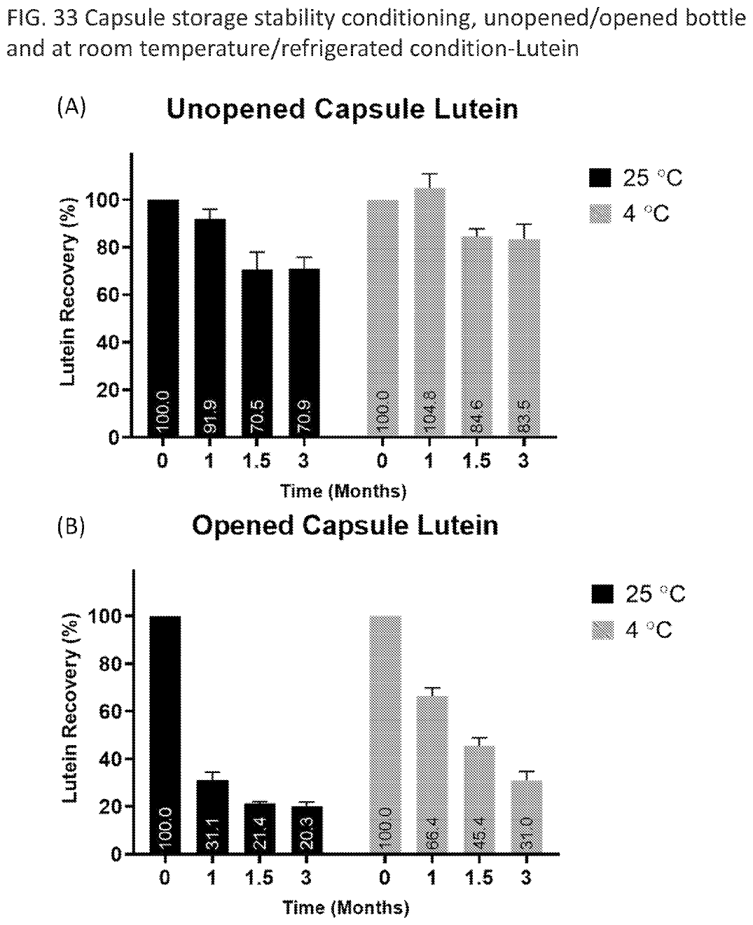

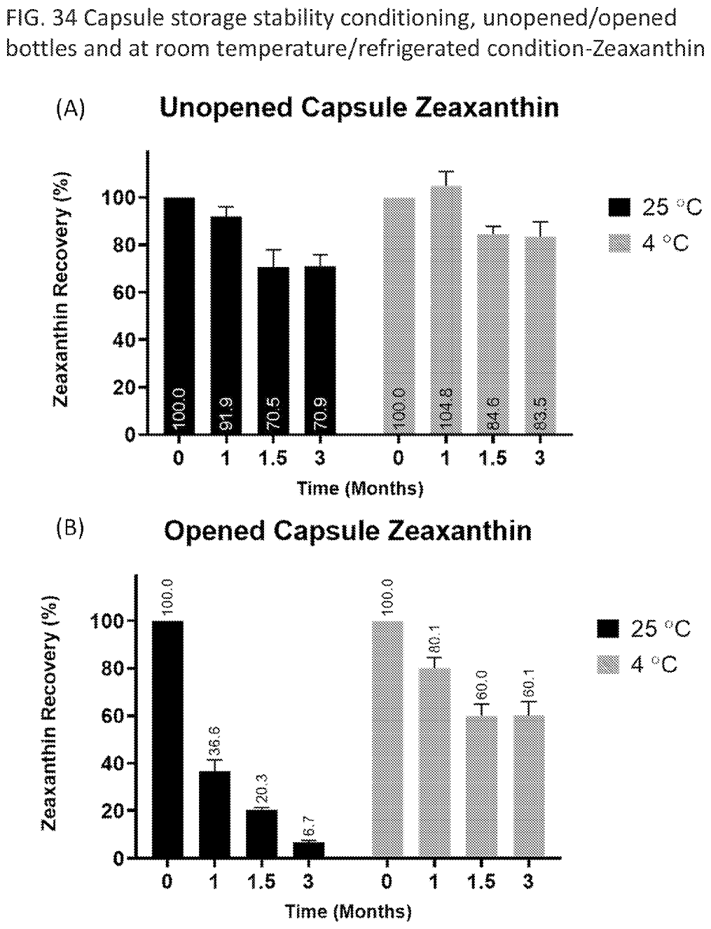

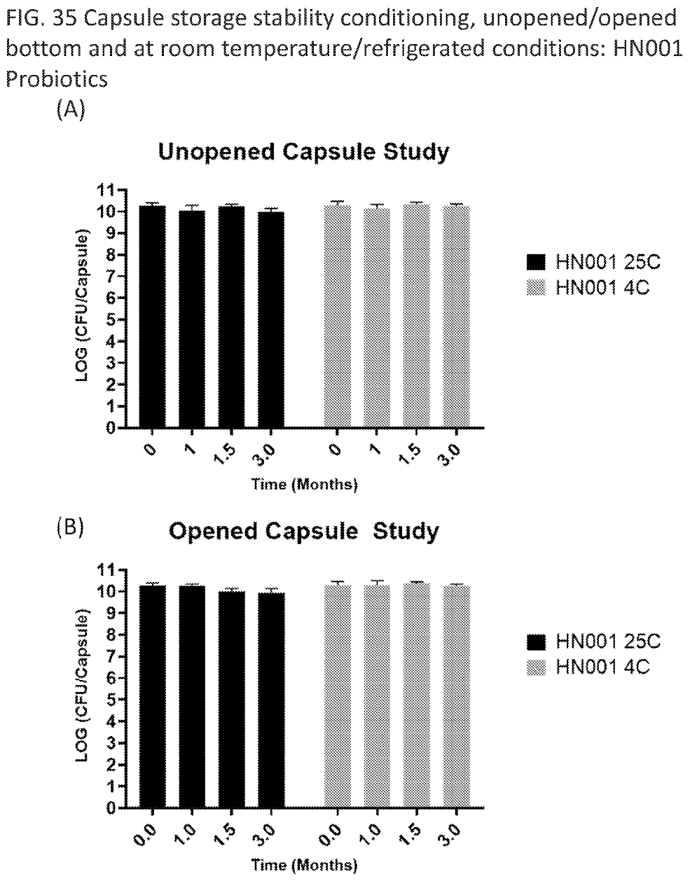

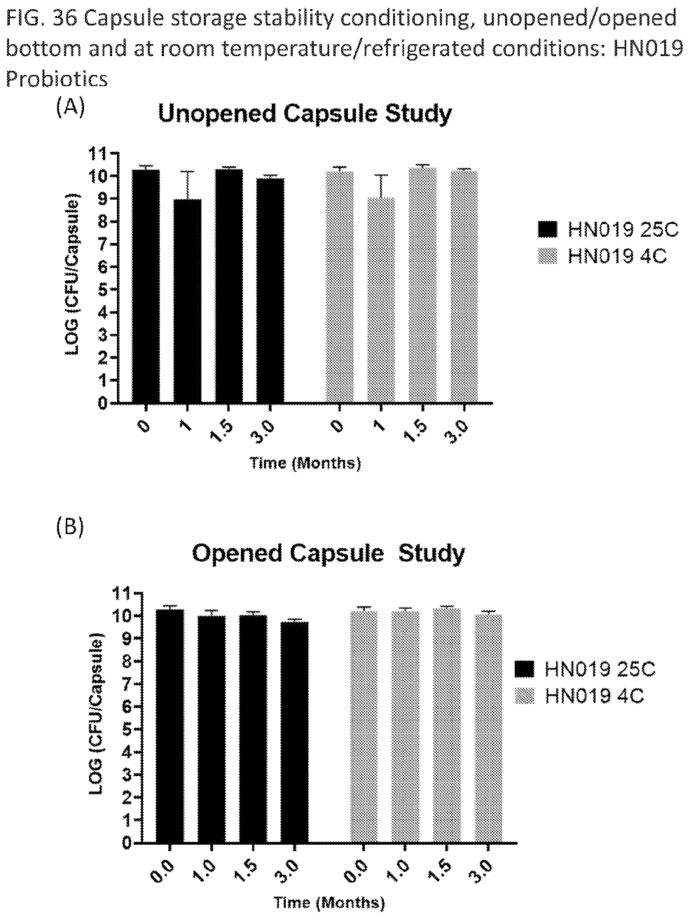

FIGS. 33-36 describe stability of microparticle lutein and zeaxanthin when co-formulated in capsules with other nutrients (e.g., probiotics, carotenoids, B vitamins, etc.). Microparticle lutein and zeaxanthin demonstrates high stability up to 3 months in capsules when stored at 4 C or 25 C in unopened conditions. Importantly, when lutein and zeaxanthin microparticles are co-formulated with probiotics in capsules, there is minimal die-off or loss of CFUs of the probiotics up to 3 months, in both opened and unopened conditions. Opened conditions expose the capsule and contents to atmospheric challenges whereas unopened conditions do not. The reason why the lutein and zeaxanthin microparticles do not induce die-off of products is because of their low water activity. There are no other lutein or zeaxanthin formulations that are compatible with capsules that can be co-formulated with probiotics because all other existing lutein and zeaxanthin formulations are high water activity and lead to rapid loss of probiotics CFUs/viability.

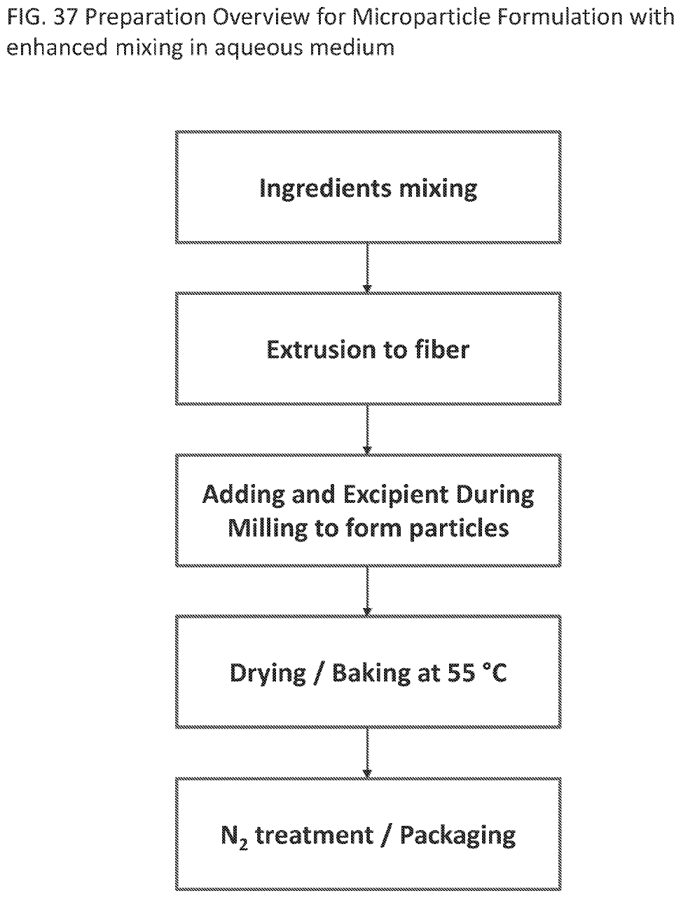

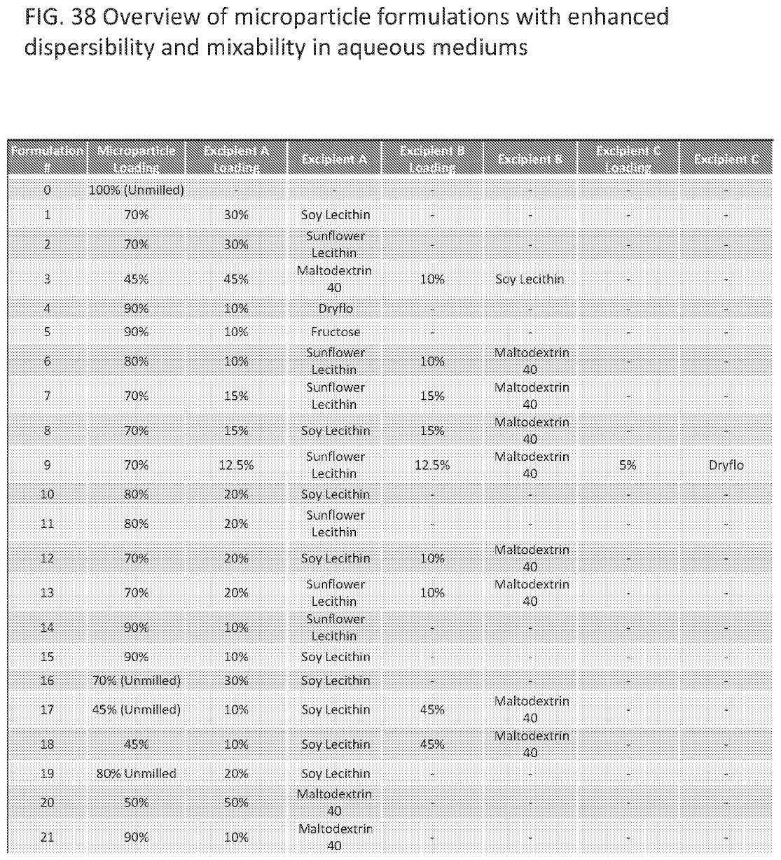

FIG. 37 shows, in a non-limiting example, a schematic of an extrusion method used to for preparation of microparticle formulation via enhanced mixing in aqueous media. The extrusion method includes ingredient mixing followed by extrusion to fiber, followed by simultaneous addition of excipient to the mixture during milling to form particles, followed by drying/baking at 55 degrees C., followed by nitrogen treatment and/or packaging. In some embodiments, the microparticle loading shown in FIG. 38 includes 20% lutein and 80% BMC.

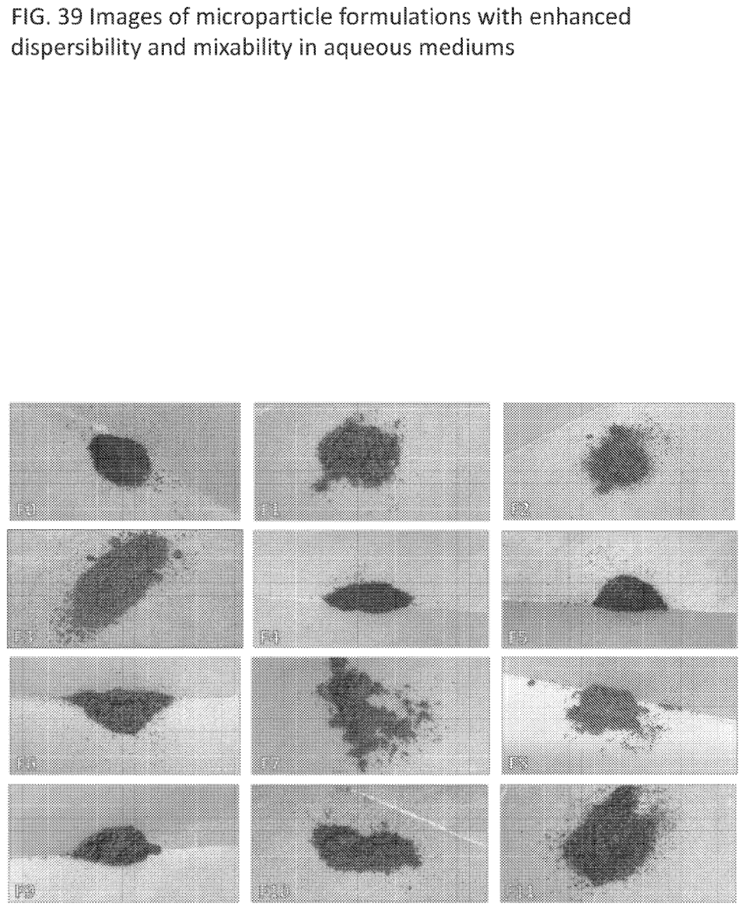

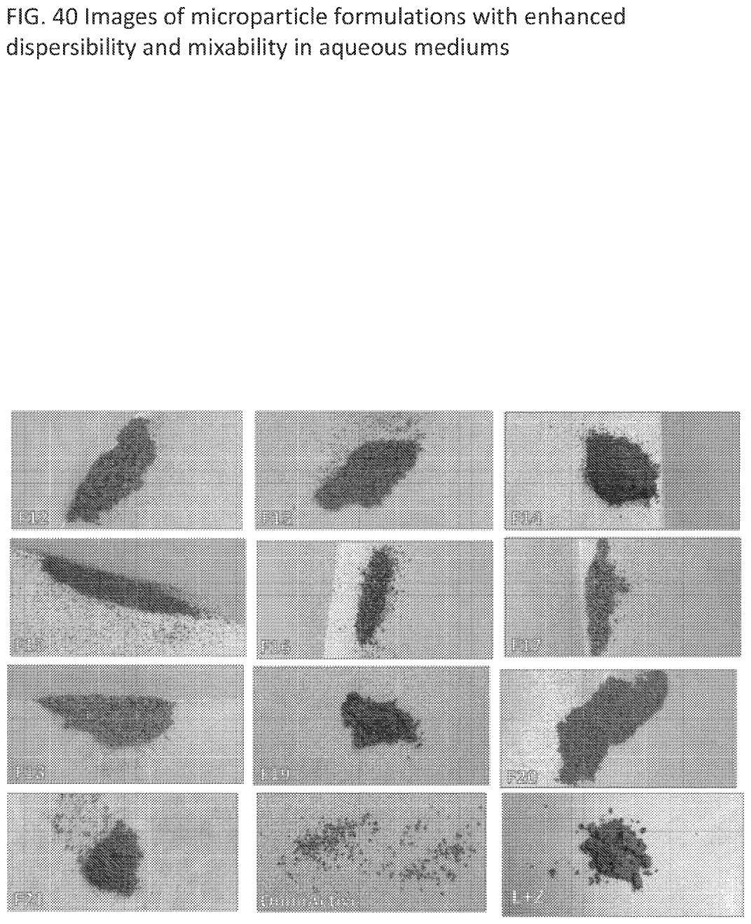

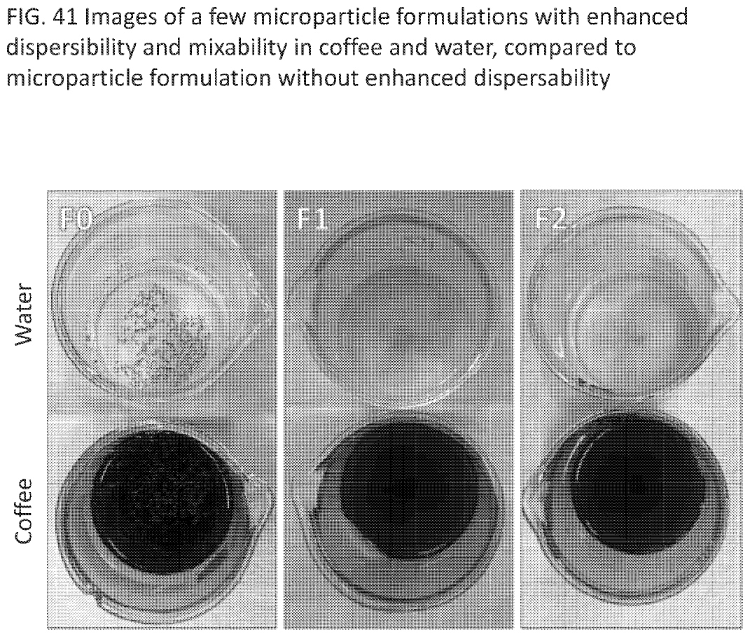

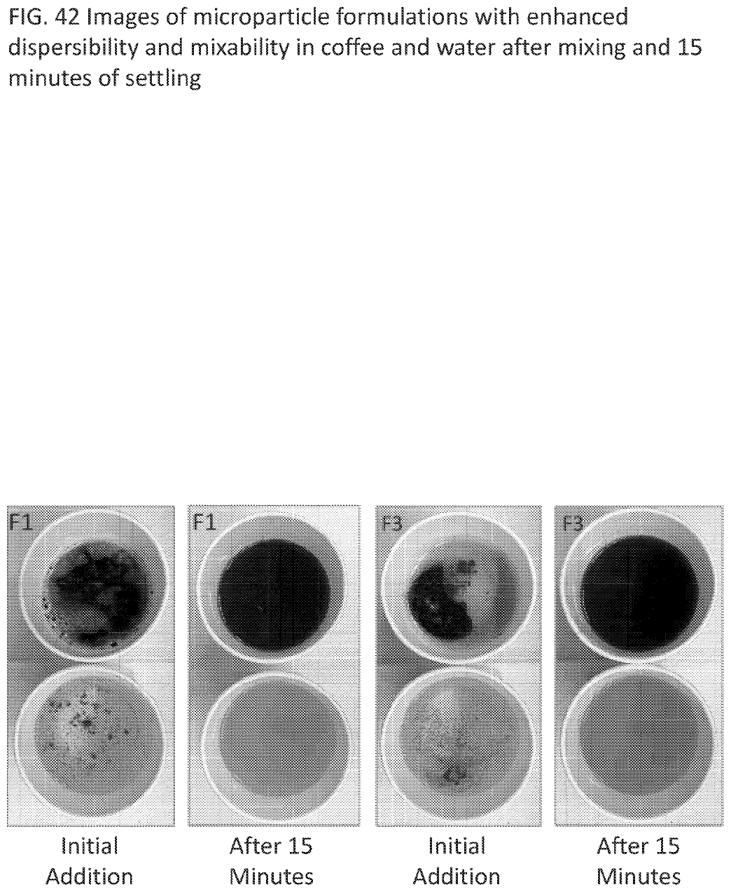

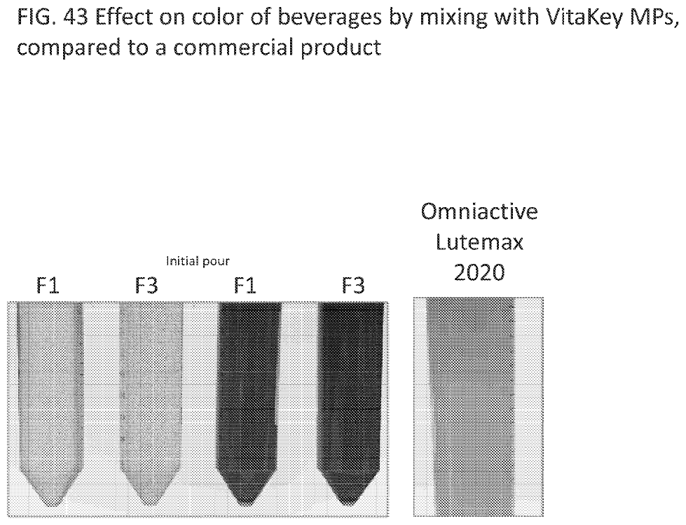

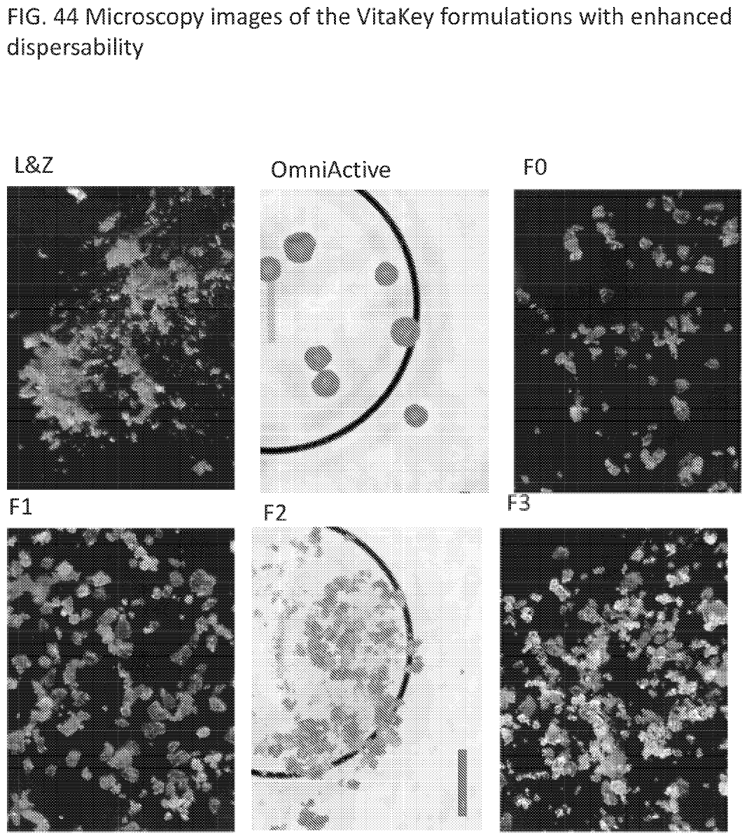

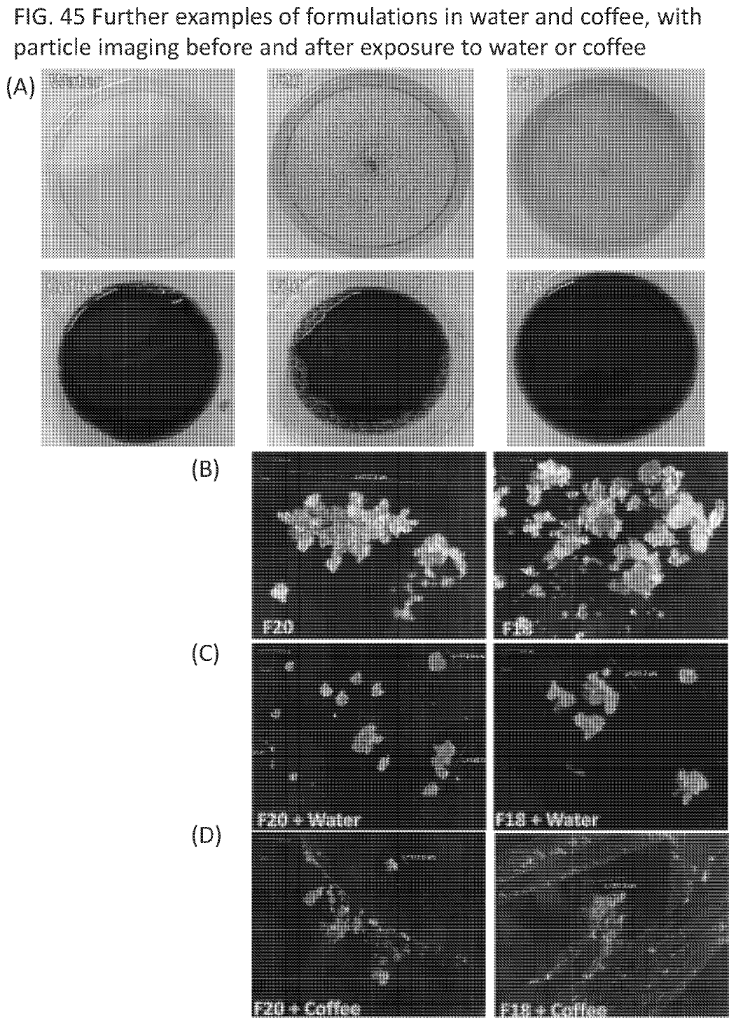

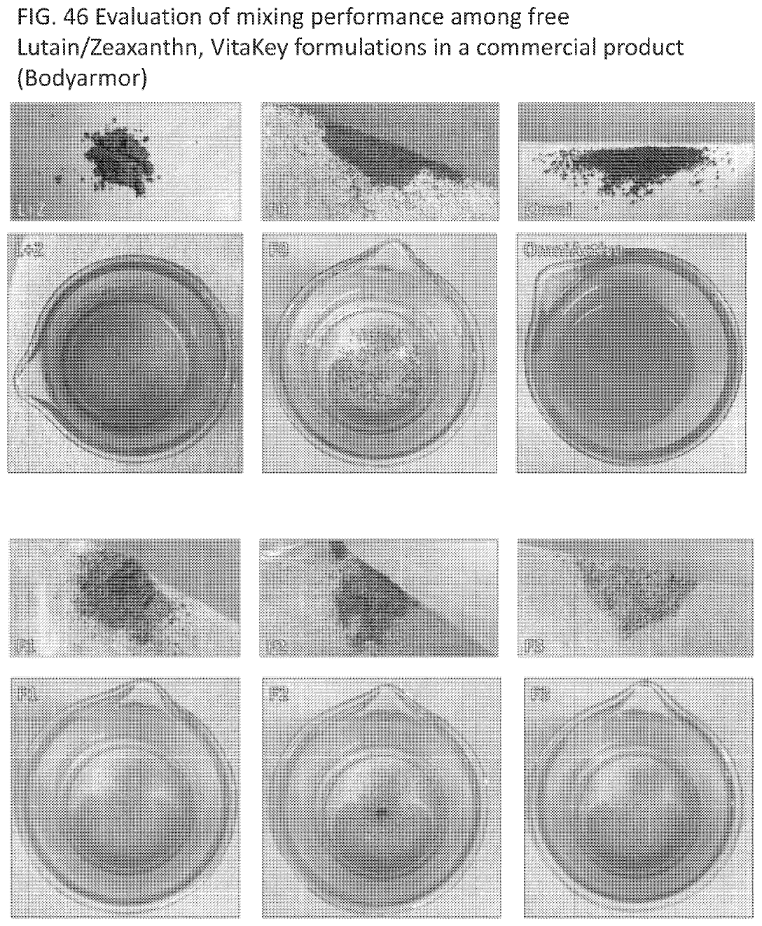

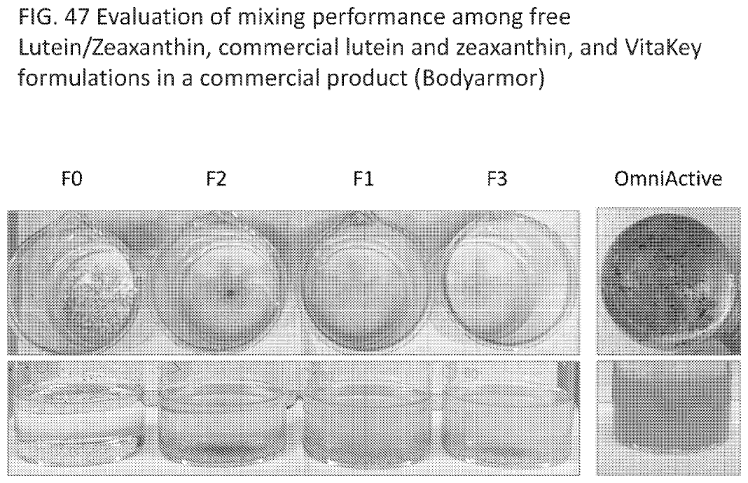

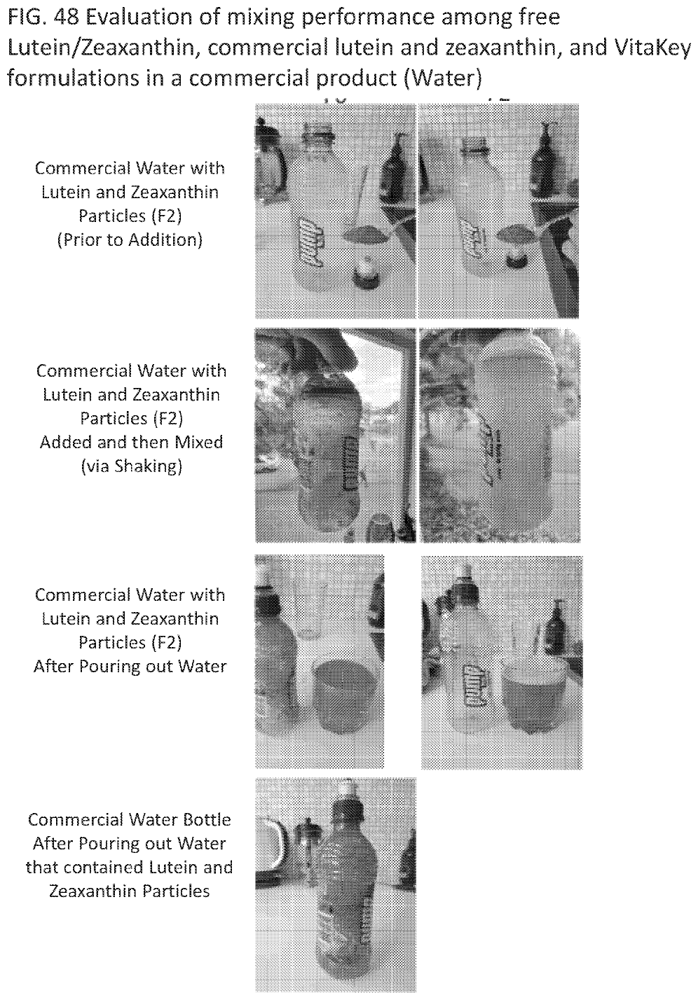

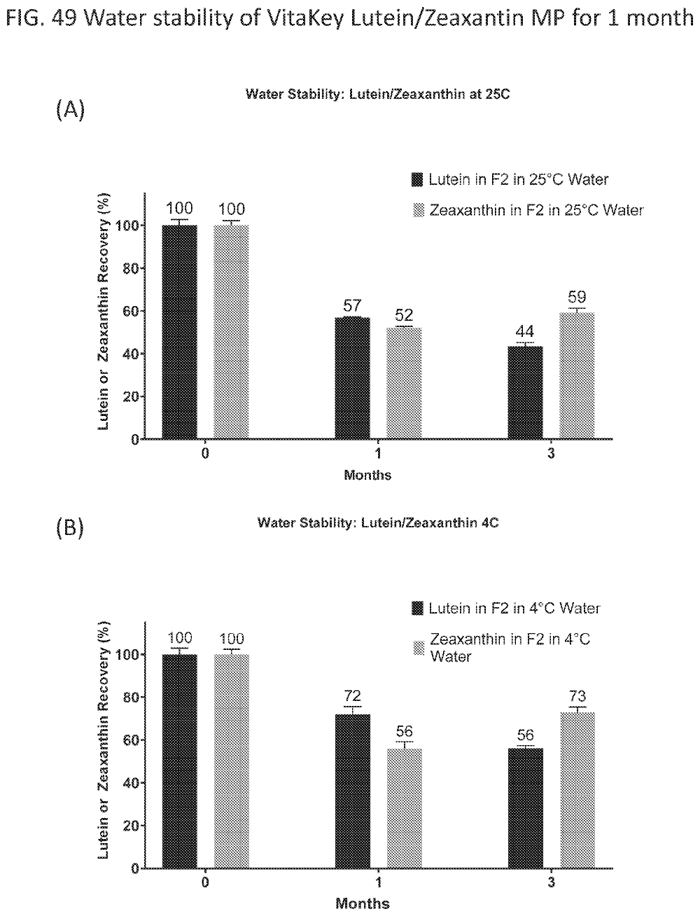

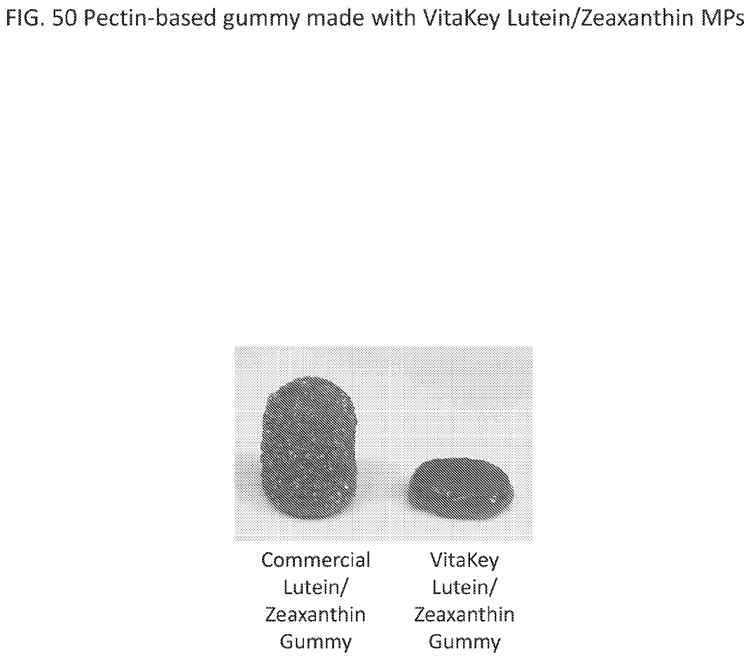

FIGS. 38-49 describe formulations of lutein and zeaxanthin microparticles that have been modified for improved dispersibility in liquid beverages. Through the addition of excipients during the milling step of manufacturing, lutein and zeaxanthin microparticles can exhibit increased mixing, dispersibility, and homogeneity in the liquid beverage. For example, FIG. 41 compares lutein and zeaxanthin microparticles without excipient (F0) to lutein and zeaxanthin microparticles with excipient (F1 and F2) which demonstrates how excipients improve mixability and dispersibility in both water and coffee. Similar results are observed in BodyArmor (FIG. 47) and water (FIG. 48). Compared to OmniActive Lutemax 2020 commercial product, lutein and zeaxanthin microparticles exhibit decreased color change and increased optical clarity (FIGS. 43, 46, 47). Microparticles also remain in intact when exposed to liquids (FIG. 45B-45D). When comparing the formulations, F1 and F3 exhibited the best performance. Given that the performance of the formulations are otherwise similar, the higher loaded formulation would be more ideal to select.

Referring to FIGS. 37-49, the microparticles and formulations described herein and shown in FIG. 37 exhibited superior dispersibility and compatibility within liquids compared to the unmilled particles (for example, F0 in FIG. 37). For example, the superior dispersibility is visually apparent in the water images of FIG. 41. F0 (unmilled) demonstrated very little dispersibility while F1 and F2 demonstrated much better dispersibility. The improved dispersibility was attributed to the concurrent adding and mixing of excipient to the mixture during milling (i.e., as contrasted to unmilled, i.e., formulation zero (F0) in FIG. 37)). Without wishing to be bound by theory, it is contemplated that the high sheer (i.e., mechanically induced) during the concurrent milling of the mixture and excipient causes the surfactant to interface with and/or physically touch the microparticles, thereby encouraging microparticle formation, and enhancing dispersibility. Without wishing to be bound by theory, it is further contemplated that concurrent mixing and milling of the excipient with the microparticle mixture results in superior dispersibility as compared to mixing of the microparticle mixture and excipient alone (i.e., without milling).

### EQUIVALENTS

While certain preferred embodiments of the present disclosure have been shown and described herein, those skilled in the art will readily appreciate that such embodiments are provided by way of example only. Numerous variations, changes, and substitutions will now occur to those skilled in the art without departing from the present embodiments. It should be understood that various alternatives to the embodiments described herein may be employed in practicing the disclosed embodiments. It is intended that the following claims define the scope of the present embodiments and that methods and structures within the scope of these claims and their equivalents be covered thereby.

### APPENDIX

The Appendices are part of the written description, and it is contemplated that information in the Appendices can be applied, used, or adapted to make or use embodiments of the invention.

### Appendix A: Quantification Method for Encapsulated Lutein and Zeaxanthin Levels in Formulation by HPLC

### 1.0 Purpose

The purpose of this Standard Analytical Method is to establish the sample preparation of Lutein and/or Zeaxanthin encapsulated microparticles, and associated analytical methods for the quantitative determination of encapsulated and free Lutein and/or Zeaxanthin by HPLC

### 2.0 Materials and Solutions

### 2.1 Solutions

2.1.1 Acetone, HPLC grade (>=99.9%): Sigma-Aldrich, Part #: 270725, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent

### 2.2 Reference Materials

| Name | Resource, Purity, and Others (Expiration Date) |
|---|---|
| Lutein (powder)* | CHENGUANG BIOTECH GROUP CO., LTD, |
| | or IOSA 75% Lutein, Exp Date: 2 years from |
| | manufacturing date (unknown) |
| Zeaxanthin (powder)* | Shandong Tianyin Biotechnology Co., LTD, |
| | or IOSA 70% Zeaxanthin, Exp Date: Unknown |
| Lutein/Zeaxanthin | Southwest Research Institute, Lutein |
| microparticles | loading (%) Zeaxanthin loading (%), |
| (powder)** | and other additives (%), Exp Date: Unknown |

*Once the manufacturer packages are opened, aliquot them in small quantity for a single use or short term use only (a few 100s mgs in glass vials), N₂ gas head them, then stored them in −80° C. freezer until use. When used, they should be equilibrated to room temperature at least for 30 minutes before opening aliquots.

### 2.3 Primary Stock Solutions, Spiking Stock Solutions, Calibration Standards/QC Samples

| Solutions | Concentration (range) |
|---|---|
| Primary Stock Solutions (PS) | Lutein at 1.333 mg/mL in Acetone (freshly prepared*, |
| | vortexed, equivalent to 1.0 mg/mL pure lutein) |
| | Zeaxanthin at 1.429 mg/mL in Acetone (freshly prepared*, |
| | vortexed, equivalent to 1.0 mg/mL pure zeaxanthin) |
| | Lutein/Zeaxanthin microparticles at 1 mg/mL in Acetone |
| | (freshly prepared*, vortexed) |
| Internal Standard Solution (IS) | N/A |
| Calibration Standard Samples | 100 μg/mL Lutein in Acetone (or other diluents****) |
| for Lutein, diluted from PS | 50 μg/mL Lutein in Acetone (or other diluents****) |
| solutions | 25 μg/mL Lutein in Acetone (or other diluents****) |
| | 12.5 μg/mL Lutein in Acetone (or other diluents****) |
| | 6.25 μg/mL Lutein in Acetone (or other diluents****) |
| | 3.125 μg/mL Lutein in Acetone (or other diluents****) |
| | 1.563 μg/mL Lutein in Acetone (or other diluents****) |
| | 0.781 μg/mL Lutein in Acetone (or other diluents****) |
| | 0.391 ug · mL Lutein in Acetone (or other diluents****) |
| Calibration Standard Samples | 100 μg/mL Zeaxanthin in Acetone (or other diluents****) |
| for Zeaxanthin, diluted from | 50 μg/mL Zeaxanthin in Acetone (or other diluents****) |
| PS solutions | 25 μg/mL Zeaxanthin in Acetone (or other diluents****) |
| | 12.5 μg/mL Zeaxanthin in Acetone (or other diluents****) |
| | 6.25 μg/mL Zeaxanthin in Acetone (or other diluents****) |
| | 3.125 μg/mL Zeaxanthin in Acetone (or other diluents****) |
| | 1.563 μg/mL Zeaxanthin in Acetone (or other diluents****) |
| | 0.781 μg/mL Zeaxanthin in Acetone (or other diluents****) |
| | 0.391 μg/mL Zeaxanthin in Acetone (or other diluents****) |
| Quality Control Samples for | N/A, to be included in the future |
| Lutein and/or Zeaxanthin, | |
| diluted from PS solutions | |

*All samples must be prepared freshly, due to instability.
** The L and Z range are subject to be changed depending on the instrument's sensitivity and maximum detection range.
*** Unknown samples (including microparticles) are dissolved in an optimal solvent such as Acetone. If other solvent is necessary to break down microparticles' polymeric matrix to release L and Z payloads, other solvent compositions can be used, including but not limited to, Dichloromethane:Acetone mixture (1:9, 2:8, 3:7, 4:6, 5:5 v/v), Methyl tert-butyl ether (MTBE):Acetone mixture (1:9, 2:8, 3:7, 4:6, 5:5 v/v), etc.
****Depending on aforementioned needs, Acetone can be replaced with other organic diluents, including but not limited to, Dichloromethane:Acetone mixture (1:9, 2:8, 3:7, 4:6, 5:5 v/v), Methyl tert-butyl ether (MTBE):Acetone mixture (1:9, 2:8, 3:7, 4:6, 5:5 v/v), etc. Calibration standards and unknown samples must be treated with the same solvents.

Below is an example of how to prepare calibration standards from stock solutions

| | | | Vol. | | | |
|---|---|---|---|---|---|---|
| | Spiking | Vol. Spiking | Acetone | Total | | |
| Spiking | Solution Conc. | solution to add | (or dilulent) | Volume | Conc. | STD/QC |
| solution ID | (μg/mL) | (μL) | (μL) | (μL) ^{b} | (μg/mL) | ID |
| Lutein stock | 1000 | 200 | 1800 | 1000 | 100 | L STD 9 |
| | (1333)* | | | | | |
| L STD9 | 100 | 1000 | 1000 | | 50 | L STD 8 |
| L STD8 | 50 | 1000 | 1000 | | 25 | L STD 7 |
| L STD 7 | 25 | 1000 | 1000 | | 12.5 | L STD 6 |
| L STD 6 | 12.5 | 1000 | 1000 | | 6.25 | L STD 5 |
| L STD 5 | 6.25 | 1000 | 1000 | | 3.125 | L STD 4 |
| L STD 4 | 3.125 | 1000 | 1000 | | 1.563 | L STD 3 |
| L STD 3 | 1.563 | 1000 | 1000 | | 0.781 | L STD 2 |
| L STD 2 | 0.781 | 1000 | 1000 | | 0.391 | L STD 1 |
| | | | 2000 | | 0 | Blank |

*Lutein PS concentration including impurity

| | Spiking | Vol. | Vol. | | | |
|---|---|---|---|---|---|---|
| | Solution | Spiking | Acetone | Total | | |
| Spiking | Conc. | solution to | (or dilulent) | Volume | Conc. | STD/QC |
| solution ID | (μg/mL) | add (μL) | (μL) | (μL) ^{b} | (μg/mL) | ID |
| Zeaxanthin | 1000 | 200 | 1800 | 1000 | 100 | Z STD 9 |
| stock | (1429)** | | | | | |
| Z STD9 | 100 | 1000 | 1000 | | 50 | Z STD 8 |
| Z STD8 | 50 | 1000 | 1000 | | 25 | Z STD 7 |
| Z STD 7 | 25 | 1000 | 1000 | | 12.5 | Z STD 6 |
| Z STD 6 | 12.5 | 1000 | 1000 | | 6.25 | Z STD 5 |
| Z STD 5 | 6.25 | 1000 | 1000 | | 3.125 | Z STD 4 |
| Z STD 4 | 3.125 | 1000 | 1000 | | 1.563 | Z STD 3 |
| Z STD 3 | 1.563 | 1000 | 1000 | | 0.781 | Z STD 2 |
| Z STD 2 | 0.781 | 1000 | 1000 | | 0.391 | Z STD 1 |
| | | | 2000 | | 0 | Blank |

**Zeaxanthin PS concentration including impurity

### 2.4 Equipments and Supplies

2.4.1 HPLC with UV/VIS detector
2.4.2 YMC C30 carotenoid column, 5 μm, 250×4.6 mm, Part #: CT99S05-2546WT
2.4.3 Borosilicate glass bottles: 500 mL, 1 L, or 2 L capacity
2.4.4 Borosilicate glass vials, 4 mL, 7 mL, and 20 mL capacity
2.4.5 Positive displacement pipettes capable of measuring volumes between 20 μL to 25 mL
2.4.6 2 mL glass HPLC vials with PTFE/Silicone septa vial caps
2.4.7 Analytical balance
2.4.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic): Pure acetone as stated in 2.1.5
3.1.3 Diluent Blank: Pure acetone as stated in 2.1.5

### 3.2 HPLC Conditions

LC system: Agilent 1260 or 1290, or equivalent
Column: YMC C30 Carotenoid, 5 μm, 250×4.6 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: 100% acetone
Gradient: Not necessary (Isocratic mode)

| Step | Time (min) | A (%) | B (%) |
|---|---|---|---|
| 0 | 0 | 1 | 99* |
| 1 | 5 | 1 | 99* |

*A:B ratio can be changed depending on needs, including but not limited to, 2:98, 3:97, 4:96, 5:95, 6:94, 7:93, 8:92, 9:91, 10:90, 11:89, etc. Please note that as the A portion increases, the retention time for Lutein and Zeaxanthin peaks are delayed, making the run time to be longer. Also, increase of A portion can separate the Lutein peak and Zeaxanthin peak farther apart.

Mobile phase flow rate: 1.0 mL/min
Injection volume: 20 μL

Run time: 5 minutes

Retention times: Lutein at 4.0 minute/Zeaxanthin at 4.5 minute

Needle Washing Solvent: TBD (tentatively, Acetone or water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |
| L Linearity Std 1 to Std 9 | 1 |
| Diluent Blank | 1 |
| Z Linearity Std 1 to Std 9 | 1 |
| Diluent Blank | 1 |
| Unknown samples (Lutein/Zeaxanthin/MPs) | 1 |
| Diluent Blank | 1 |
| L Linearity Std 1 to Std 9 | 1 |
| Diluent Blank | 1 |
| Z Linearity Std 1 to Std 9 | 1 |
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |

### 3.4 Data Analysis

Note: Refer to Attachment 5.1.1 for example integrated chromatograms.
3.4.1 System Suitability
   3.4.1.1 The chromatographic profile of the 100 μg/mL L and Z Standards (or any other concentrations chosen as quality control purpose, determined using fresh L and Z) are comparable to the chromatogram determined using very fresh L and Z.
   3.4.1.2% RSD≤3 for the main peak areas of the six 100 μg/mL L and Z Standard (or any other concentrations chosen as quality control purpose, determined using fresh L and Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L and Z standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²≥0.99.
   3.4.2.5 The percent recovery of standard levels must be within 90% to 110% of its label claim.
3.4.3 Lutein, Zeaxanthin Concentrations in samples
   3.4.3.1 Using the standard curve values for L and Z, calculate the concentration of L and Z using the following equation:

$$L or Z ( \frac{mg}{mL} ) = \frac{{A}_{Test} - y intercept}{slop e} \times DF \times 0.001$$

Aₜₑₛₜ=Average main peak area of either L or Z
DF=dilution factor of the test sample.
0.001=convert μg/mL to mg/mL
3.4.3.2 Calculate the percent recovery of the test sample as:

$$\frac{Sample L or Z concentration ( \frac{mg}{mL} )}{Label Claim of L or Z test sample ( \frac{mg}{mL} )} \times 100$$

3.4.3.3 Calculate the percent amount of L and Z loading as:

$$\frac{Sample L or Z concentration ( \frac{mg}{mL} )}{MP concentration ( \frac{mg}{mL} )} \times 100$$

3.4.3.4 The percent recovery of each test sample must be within 85% to 110% of its label claim.

### 3.5 Reporting

3.5.1 Report the percent encapsulated L/Z in the test sample to one decimal place (e.g. 96.5%).
3.5.2 Report the percent free L/Z in the test sample to one decimal place (e.g. 3.5%).
   4.0

### 5.0 Attachments

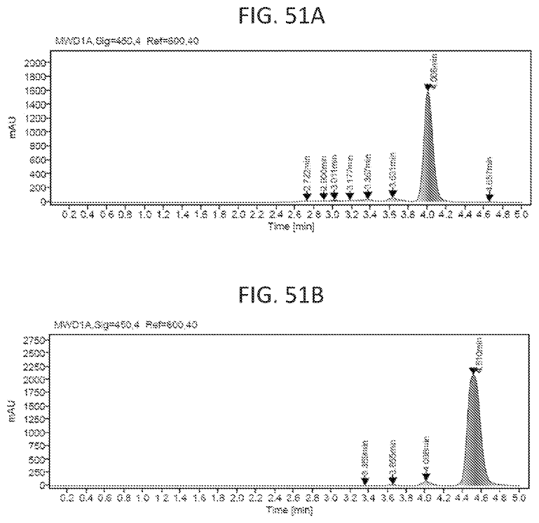

5.1.1 Examples of chromatograms are presented in FIG. 51A and FIG. 51B, wherein FIG. 51A show shows Lutein at a concentration of 100 μg/mL in acetone having a main peak at 4.0 minute retention time and FIG. 51B shows Zeaxanthin at a concentration of 100 μg/mL in acetone, main peak at 4.5 minute retention time.

### Appendix B: Quantification Method for Lutein and Zeaxanthin Levels from Powder Milk Solution by HPLC

### 1.0 Purpose

The purpose of this Standard Operating Procedure is to establish the sample preparation of Lutein and/or Zeaxanthin encapsulated microparticles, extracted from powder milk solution and associated analytical methods for the quantitative determination of Lutein and/or Zeaxanthin by HPLC

### 2.0 Materials and Solutions

### 2.1 Solutions

2.1.1 Acetone, HPLC grade (>=99.9%): Sigma-Aldrich, Part #: 270725, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent
2.1.6 Ethanol, 200 proof: Decon Labs, Part #: 22-032-601 (UN1170), or equivalent, or HPLC grade, Fisher Scientific, Part #: AC611050040, or equivalent
2.1.7 Dichloromethane, ACS reagent, Part #: 32222 (UN1593) or equivalent

### 2.2 Reference Materials

| Name | Resource, Purity, and Others (Expiration Date) |
|---|---|
| Lutein (powder)* | CHENGUANG BIOTECH GROUP CO., LTD, or |
| | IOSA 75% Lutein, Exp Date: 2 years from |
| | manufacturing date (unknown) |
| Zeaxanthin | Shandong Tianyin Biotechnology Co., LTD, |
| (powder)* | or IOSA 70% Zeaxanthin, Exp Date: Unknown |
| Lutein/Zeaxanthin | Southwest Research Institute, Lutein |
| microparticles | loading (%) Zeaxanthin loading (%), |
| (powder)** | and other ingredients (%), Exp Date: TBD |
| Powder milk | Fonterra, High fat milk powder from whole milk, |
| | Lot: 23137233, Stock ID: 659 or equivalent |

*Once the manufacturer packages of Lutein and Zeaxanthin are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein and Zeaxanthin extraction from powder milk by liquid-liquid extraction method
2.3.1 Weigh powder milk (e.g. 2 g)
2.3.2 Add certain amount of DI water to make 10% (or any other concentrations depending on needs) (e.g. 2 g powder milk in 18 mL water to make about 20 mL 10% powder milk solution) and use this solution as matrix stock solution
2.3.3 Weight L/Z encapsulated microparticles
2.3.4 Add powder milk solution to the L/Z microparticles to make the concentration of interest
2.3.5 Vortex and sonicate the powder milk solutions containing L/Z microparticles
2.3.6 Immediately after the agitation, transfer 0.5 mL of the powder milk solution into 2 mL Eppendorf vial
2.3.7 Add 0.5 mL of Ethanol into the powder milk solution to make 1 mL volume (the volume can be increased to make better dissolution if necessary) and vortex to cause partial dissolution of L/Z microparticles and to induce release of L/Z from the powder milk contents and MP matrix.
2.3.8 Add 0.5 mL of dichloromethane into the suspension and vortex (the volume can be increased to make better dissolution and better separation of L and Z into the DCM rich bottom layer, if necessary)
2.3.9 Centrifuge the tube at 10000 RPM for 2 min (the centrifuge speed and time can be adjusted to obtain better extraction).
   2.3.9.1 It will form three layers, two liquid layers (phase separation) and a solid layer of powder milk contents in between those two liquid layers.
   2.3.9.2 The bottom liquid layer (DCM rich layer) should have majority of orange color from L and Z.
2.3.10 Prepare 900 μL of acetone in HPLC vials
2.3.11 Take 100 μL of the bottom liquid layer from the centrifuged mixture sample using a pipet carefully not to interrupt the bottom solid pellet and top aqueous layer
2.3.12 Add it into the prepared 900 μL acetone in HPLC vial (1/10× dilution, Dilution factor 10). Be careful not to lose the solution by dripping from the pipet tip, due to the low surface tension of DCM rich solution
2.3.13 Cap the 1/10× diluted extract
2.3.14 Vortex the vial
2.3.15 Inject into HPLC following the HPLC method defined in the later section
2.3.16 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Lutein and Zeaxanthin powder
2.4.3 Suspend the powder in the powder milk to make a suspension at 1.333 mg/mL for Lutein (equivalent to 1.0 mg/mL of pure Lutein) and 1.429 mg/mL for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: powder milk 1:1 mixture (at least 40 mL)
2.4.7 Prepare 10 empty 2 mL Eppendorf tubes for each Lutein and Zeaxanthin
2.4.8 Fill 1 mL of EtOH: powder milk mixture into 9 of them (1 empty and 9 with 1 mL EtOH)
2.4.9 Vortex the Lutein stock suspension (or Zeaxanthin stock suspension) and immediately after the agitation, transfer 1.0 mL of the suspension into one empty tube and one of 9 tubes with 1.0 mL EtOH: powder milk mixture (making 1 mL as the same concentration and 2 mL of 1/2× dilution)
2.4.10 After agitation, take out 1 mL from 1/2× dilution and add into the next 1 mL pure EtOH: powder milk mixture making second 1/2× dilution. Continue this 1/2× serial dilutions into the next tube to make 10 concentration levels for each Lutein and Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L and Z into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of powder milk contents in between those two liquid layers.
   2.4.13.2 The bottom liquid layer (DCM rich layer) should have majority of orange color from L and Z.
2.4.14 Prepare 900 μL of acetone in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL acetone in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in pure milk |
| | (freshly prepared*, vortexed, |
| | equivalent to 1.0 mg/mL pure lutein) |
| | Zeaxanthin at 1.429 mg/mL in pure |
| | milk (freshly prepared*, vortexed, |
| | equivalent to 1.0 mg/mL pure |
| | zeaxanthin) |
| | L and Z are not soluble and suspendable |
| | in milk. So, serial dilution to prepare |
| | for the calibration standards should be |
| | performed after adding EtOH (powder |
| | milk sample:EtOH 1:1) and diluted into |
| | EtOH:powder milk (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.25 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 μg/mL Zeaxanthin |
| | 0.195 ug/mL Zeaxanthin |
| Quality Control Samples for | N/A, to be included in the future |
| Lutein and/or Zeaxanthin, | |
| diluted from PS solutions | |

*All samples must be prepared freshly, due to instability.
** The L and Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:powder | Total | | DCM | | | Nominal |
| Spiking | Conc. | to add | milk mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

| | | Vol. | | | | | | | |
|---|---|---|---|---|---|---|---|---|---|
| | Spiking | Spiking | Vol. | | | Vol. | | | |
| | Solution | solution | EtOH:powder | Total | | DCM | | | Nominal |
| Spiking | Conc. | to add | milk mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

### 2.5 Equipment and Supplies

2.5.1 HPLC with UV/VIS detector
2.5.2 YMC C30 carotenoid column, 5 μm, 250×4.6 mm, Part #: CT99S05-2546WT
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic): Pure acetone as stated in 2.1.5
3.1.3 Diluent Blank: Pure acetone as stated in 2.1.5

### 3.2 HPLC Conditions

LC system: Agilent 1260 or 1290, or equivalent
Column: YMC C30 Carotenoid, 5 μm, 250×4.6 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: 100% acetone
Gradient: Not necessary (Isocratic mode)

| Step | Time (min) | A (%) | B (%) |
|---|---|---|---|
| 0 | 0 | 1 | 99* |
| 1 | 5 | 1 | 99* |

*A:B ratio can be changed depending on needs, including but not limited to, 2:98, 3:97, 4:96, 5:95, 6:94, 7:93, 8:92, 9:91, 10:90, 11:89, etc. Please note that as the A portion increases, the retention time for Lutein and Zeaxanthin peaks are delayed, making the run time longer. Also, increase of A portion can separate the Lutein peak and Zeaxanthin peak farther apart.

Mobile phase flow rate: 1.0 mL/min
Injection volume: 20 μL
Run time: 5 minutes
Needle Washing Solvent: TBD (tentatively, Acetone or water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |
| L calibration Std 1 to Std 10 | 1 |
| Diluent Blank | 1 |
| Z calibration Std 1 to Std 10 | 1 |
| Diluent Blank | 1 |
| Unknown samples (Lutein/Zeaxanthin/MPs extract | 1 |
| from milk) | |
| Diluent Blank | 1 |
| L calibration Std 1 to Std 9 | 1 |
| Diluent Blank | 1 |
| Z calibration Std 1 to Std 9 | 1 |
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |

### 3.4 Data Analysis

Note: Refer to Attachment 5.1.1 for example integrated chromatograms and recovery.
3.4.1 System Suitability
   3.4.1.1 The chromatographic profile of the 100 μg/mL L and Z Standards (or any other concentrations chosen as quality control purpose, determined using fresh L and Z) are comparable to the chromatogram determined using very fresh L and Z.
   3.4.1.2% RSD≤3 for the main peak areas of the six 100 μg/mL L and Z Standard (or any other concentrations chosen as quality control purpose, determined using fresh L and Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L and Z standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L and Z, calculate the concentration of L and Z using the following equation:

$$L or Z ( \frac{mg}{mL} ) = \frac{{A}_{Test} - y intercept}{slop e} \times DF \times 0.001$$

Aₜₑₛₜ=Average main peak area of either L or Z
DF=dilution factor of the test sample.
0.001=convert μg/mL to mg/mL
3.4.3.2 Calculate the percent recovery of the test sample as:

$$\frac{Extracted L or Z concentration ( \frac{mg}{mL} )}{Neat condition control L or Z test sample ( \frac{mg}{mL} )} \times 100$$

3.4.3.3 Calculate the percent amount of L and Z loading as:

$$\frac{Determined sample L or Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L/Z in the test sample to one decimal place (e.g. 96.5%).
   3.5.2 Report the percent free L/Z in the test sample to one decimal place (e.g.
3.5%).
   4.0

### 5.0 Attachments

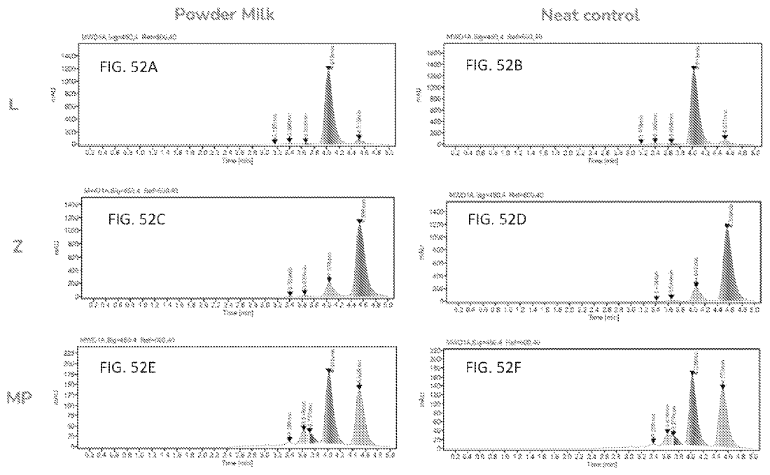

5.1.1 Preliminary extraction results and recovery are presented in FIGS. 52A-52F and summarized in the table below.

| Powder Milk | AUC (P. milk) | AUC (Neat) | Recovery (%) |
|---|---|---|---|
| Lutein | 10707.1 | 11845.2 | 90.4 |
| Zeaxanthin | 11610.9 | 12035.5 | 96.5 |
| P81 (L) | 1437.6 | 1461.6 | 98.4 |
| P81 (Z) | 1278.7 | 1318.3 | 97.0 |

### Appendix C: Quantification Method for Lutein and Zeaxanthin Levels from Milk by HPLC

### 1.0 Purpose

The purpose of this Standard Operating Procedure is to establish the sample preparation of Lutein and/or Zeaxanthin encapsulated microparticles, extracted from milk and associated analytical methods for the quantitative determination of Lutein and/or Zeaxanthin by HPLC

### 2.0 Materials and Solutions

### 2.1 Solutions

2.1.1 Acetone, HPLC grade (>=99.9%): Sigma-Aldrich, Part #: 270725, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent
2.1.6 Ethanol, 200 proof: Decon Labs, Part #: 22-032-601 (UN1170), or equivalent, or HPLC grade, Fisher Scientific, Part #: AC611050040, or equivalent
2.1.7 Dichloromethane, ACS reagent, Part #: 32222 (UN1593) or equivalent
2.1.8 Whole milk or Lutein/Zeaxanthin (free or microparticles) fortified milk

### 2.2 Reference Materials

| | Resource, Purity, and Others |
|---|---|
| Name | (Expiration Date) |
| Lutein (powder)* | CHENGUANG BIOTECH GROUP CO., LTD, or |
| | IOSA |
| | 75% Lutein, Exp Date: 2 years from |
| | manufacturing date (unknown) |
| Zeaxanthin | Shandong Tianyin Biotechnology Co., |
| (powder)* | LTD, or IOSA |
| | 70% Zeaxanthin, Exp Date: Unknown |
| Lutein/Zeaxanthin | Southwest Research Institute, Lutein |
| microparticles | loading (%) Zeaxanthin loading (%), |
| (powder)** | and other ingredients (%), Exp Date: TBD |

*Once the manufacturer packages are opened, aliquot them in small quantity for a single use or short term use only (a few 100 s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein and Zeaxanthin extraction from milk by liquid-liquid extraction method
2.3.1 For regular milk, weigh certain mg of L/Z encapsulated microparticles and spike them into the milk
2.3.2 For L/Z microparticle fortified milk product, use the milk product directly
2.3.3 Vortex and sonicate the milk solutions containing L/Z microparticles
2.3.4 Immediately after the agitation, transfer 0.5 mL of the milk solution into 2 mL Eppendorf vial
2.3.5 Add 0.5 mL of Ethanol into the milk solution to make 1 mL volume (the volume can be increased to make better dissolution if necessary) and vortex to cause partial dissolution of L/Z microparticles and to induce release of L/Z from milk contents and MP matrix.
2.3.6 Add 0.5 mL of dichloromethane into the suspension and vortex (the volume can be increased to make better dissolution and better separation of L and Z into the DCM rich bottom layer, if necessary)
2.3.7 Centrifuge the tube at 10000 RPM for 2 min (the centrifuge speed and time can be adjusted to obtain better extraction).
   2.3.7.1 It will form three layers, two liquid layers (phase separation) and a solid layer of milk contents in between those two liquid layers.
   2.3.7.2 The bottom liquid layer (DCM rich layer) should have majority of orange color from L and Z.
2.3.8 Prepare 900 μL of acetone in HPLC vials
2.3.9 Take 100 μL of the bottom liquid layer from the centrifuged mixture sample using a pipet carefully not to interrupt the bottom solid pellet and top aqueous layer
2.3.10 Add it into the prepared 900 μL acetone in HPLC vial (1/10× dilution, Dilution factor 10). Be careful not to lose the solution by dripping from the pipet tip, due to the low surface tension of DCM rich solution
2.3.11 Cap the 1/10× diluted extract
2.3.12 Vortex the vial
2.3.13 Inject into HPLC following the HPLC method defined in the later section
2.3.14 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3 2.4.2 Weigh certain mg (at least 3 mg) of the fresh (or properly stored) Lutein and Zeaxanthin powder
2.4.3 Suspend the powder in pure milk to make a suspension at 1.333 mg/mL for Lutein (equivalent to 1.0 mg/mL of pure Lutein) and 1.429 mg/mL for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: pure milk 1:1 mixture (at least 40 mL)
2.4.7 Prepare 10 empty 2 mL Eppendorf tubes for each Lutein and Zeaxanthin
2.4.8 Fill 1 mL of EtOH: pure milk mixture into 9 of them (1 empty and 9 with 1 mL EtOH)
2.4.9 Vortex the Lutein stock suspension (or Zeaxanthin stock suspension) and immediately after the agitation, transfer 1.0 mL of the suspension into one empty tube and one of 9 tubes with 1.0 mL EtOH: pure milk mixture (making
1 mL as the same concentration and 2 mL of 1/2× dilution)
2.4.10 After agitation, take out 1 mL from 1/2× dilution and add into the next 1 mL pure EtOH: pure milk mixture making second 1/2× dilution. Continue this 1/2×serial dilutions into the next tube to make 10 concentration levels for each Lutein and Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L and Z into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of milk contents in between those two liquid layers.
   2.4.13.2 The bottom liquid layer (DCM rich layer) should have majority of orange color from L and Z.
2.4.14 Prepare 900 μL of acetone in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL acetone in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in pure milk (freshly prepared*, |
| | vortexed, equivalent to 1.0 mg/mL pure lutein) |
| | Zeaxanthin at 1.429 mg/mL in pure milk (freshly prepared*, |
| | vortexed, equivalent to 1.0 mg/mL pure zeaxanthin) |
| | L and Z are not soluble and suspendable in milk. So, serial |
| | dilution to prepare for the calibration standards should be |
| | performed after adding EtOH (milk sample:EtOH 1:1) and |
| | diluted into EtOH:pure milk (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.25 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 μg/mL Zeaxanthin |
| | 0.195 ug/mL Zeaxanthin |
| Quality Control Samples for | N/A, to be included in the future |
| Lutein and/or Zeaxanthin, | |
| diluted from PS solutions | |

*All samples must be prepared freshly, due to instability.
** The L and Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:pure | Total | | DCM | | | Nominal |
| Spiking | Conc. | to add | milk mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

| | | Vol. | Vol. | | | | | | |
|---|---|---|---|---|---|---|---|---|---|
| | Spiking | Spiking | EtOH:pure | | | Vol. | | | |
| | Solution | solution | milk | Total | | DCM | | | Nominal |
| Spiking | Conc. | to add | mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

### 2.5 Equipments and Supplies

2.5.1 HPLC with UV/VIS detector
2.5.2 YMC C30 carotenoid column, 5 μm, 250×4.6 mm, Part #: CT99S05-2546WT
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic): Pure acetone as stated in 2.1.5
3.1.3 Diluent Blank: Pure acetone as stated in 2.1.5

### 3.2 HPLC Conditions

LC system: Agilent 1260 or 1290, or equivalent
Column: YMC C30 Carotenoid, 5 μm, 250×4.6 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: 100% acetone
Gradient: Not necessary (Isocratic mode)

| Step | Time (min) | A (%) | B (%) |
|---|---|---|---|
| 0 | 0 | 1 | 99* |
| 1 | 5 | 1 | 99* |

*A:B ratio can be changed depending on needs, including but not limited to, 2:98, 3:97, 4:96, 5:95, 6:94, 7:93, 8:92, 9:91, 10:90, 11:89, etc. Please note that as the A portion increases, the retention time for Lutein and Zeaxanthin peaks are delayed, making the run time longer. Also, increase of A portion can separate the Lutein peak and Zeaxanthin peak farther apart.

Mobile phase flow rate: 1.0 mL/min
Injection volume: 20 μL
Run time: 5 minutes
Retention times: Lutein at 4.0 minute/Zeaxanthin at 4.5 minute
Needle Washing Solvent: TBD (tentatively, Acetone or water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |
| L calibration Std 1 to Std 10 | 1 |
| Diluent Blank | 1 |
| Z calibration Std 1 to Std 10 | 1 |
| Diluent Blank | 1 |
| Unknown samples (Lutein/Zeaxanthin/MPs extract | 1 |
| from milk) | |
| Diluent Blank | 1 |
| L calibration Std 1 to Std 9 | 1 |
| Diluent Blank | 1 |
| Z calibration Std 1 to Std 9 | 1 |
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |

### 3.4 Data Analysis

Note: Refer to Attachment 1 for example integrated chromatograms and recovery
3.4.1 System Suitability
   3.4.1.1 The chromatographic profile of the 100 μg/mL L and Z Standards (or any other concentrations chosen as quality control purpose, determined using fresh L and Z) are comparable to the chromatogram determined using very fresh L and Z.
   3.4.1.2% RSD≤3 for the main peak areas of the six 100 μg/mL L and Z Standard (or any other concentrations chosen as quality control purpose, determined using fresh L and Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L and Z standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L and Z, calculate the concentration of L and Z using the following equation:

$$L or Z ( \frac{mg}{mL} ) = \frac{{A}_{Test} - y intercept}{slope} \times DF \times 0.001$$

Aₜₑₛₜ=Average main peak area of either L or Z
DF=dilution factor of the test sample.
0.001=convert μg/mL to mg/mL
3.4.3.2 Calculate the percent recovery of the test sample as:

$$\frac{Extracted L or Z concentration ( \frac{mg}{mL} )}{Neat condition control L test sample ( \frac{mg}{mL} )} \times 100$$

3.4.3.3 Calculate the percent amount of L and Z loading as:

$$\frac{Determined sample L or Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L/Z in the test sample to one decimal place (e.g. 96.5%).
3.5.2 Report the percent free L/Z in the test sample to one decimal place (e.g. 3.5%).
   4.0

### 5.0 Attachments

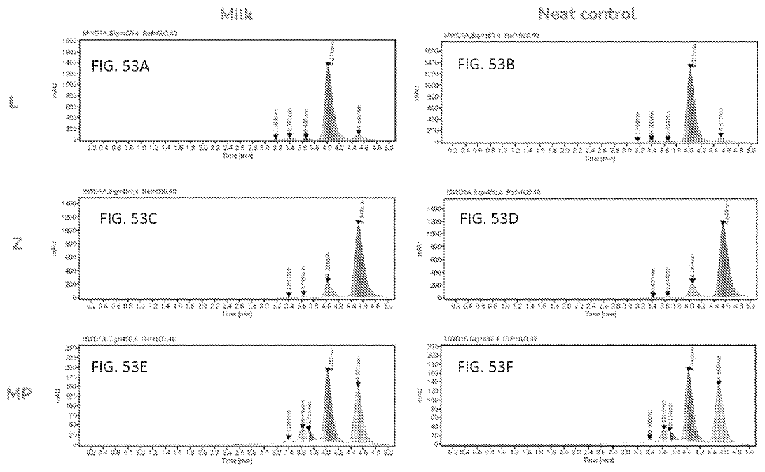

5.1.1 Preliminary extraction results and recovery are presented in FIG. 53A-FIG. 53F and summarized in the table below.

| Regular Milk | AUC (milk) | AUC (Neat) | Recovery (%) |
|---|---|---|---|
| Lutein | 12145.4 | 11845.2 | 102.5 |
| Zeaxanthin | 11449.8 | 12035.5 | 95.1 |
| P81 (L) | 1541.9 | 1461.6 | 105.5 |
| P81 (Z) | 1395.6 | 1318.3 | 105.9 |

### Appendix D: Quantification Method for Lutein and Zeaxanthin Levels from Probiotic Capsules by HPLC

### 1.0 Purpose

The purpose of this Standard Operating Procedure is to establish the sample preparation of Lutein and/or Zeaxanthin encapsulated microparticles, extracted from probiotic capsule product with other ingredients and associated analytical methods for the quantitative determination of Lutein and/or Zeaxanthin by HPLC

### 2.0 Materials and Solutions

### 2.1 Solutions

2.1.1 Acetone, HPLC grade (>=99.9%): Sigma-Aldrich, Part #: 270725, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent
2.1.6 Ethanol, 200 proof: Decon Labs, Part #: 22-032-601 (UN1170), or equivalent, or HPLC grade, Fisher Scientific, Part #: AC611050040, or equivalent
2.1.7 Dichloromethane, ACS reagent, Part #: 32222 (UN1593) or equivalent

### 2.2 Reference Materials

| Name | Resource, Purity, and Others (Expiration Date) |
|---|---|
| Lutein (powder)* | CHENGUANG BIOTECH GROUP CO., LTD, |
| | or IOSA |
| | 75% Lutein, Exp Date: 2 years from |
| | manufacturing date (unknown) |
| Zeaxanthin (powder)* | Shandong Tianyin Biotechnology Co., LTD, or |
| | IOSA |
| | 70% Zeaxanthin, Exp Date: Unknown |
| Lutein/Zeaxanthin | Southwest Research Institute, Lutein loading (%) |
| microparticles | Zeaxanthin loading (%), and other ingredients |
| (powder)** | (%), Exp Date: TBD |
| Probiotics Capsule | Manufacturer: TBD |
| product containing L/Z | Lutein content (% or mg) Zeaxanthin content |
| microparticles | (% or mg), and other ingredients (% or mg), Exp |
| | Date: TBD |

*Once the manufacturer packages are opened, aliquot them in small quantity for a single use or short term use only (a few 100 s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein and Zeaxanthin extraction from probiotics capsule content mixture by liquid-liquid extraction method
2.3.1 Open capsule and collect powder contents in a separate vial
2.3.2 Weigh certain mg of the collected capsule content powder
2.3.3 Suspend the powder in DI water to make a suspension at 20 mg/mL concentration
2.3.4 Vortex and sonicate the suspension
2.3.5 Immediately after the agitation, transfer 0.5 mL of the suspension into 2 mL Eppendorf vial
2.3.6 Add 0.5 mL of Ethanol into the suspension and vortex to form partially dissolved suspension to make 1 mL volume (the volume can be increased to make better dissolution if necessary)
2.3.7 Add 0.5 mL of dichloromethane into the suspension and vortex (the volume can be increased to make better dissolution and better separation of L and Z into the DCM rich bottom layer, if necessary)
2.3.8 Centrifuge the tube at 10000 RPM for 2 min (the centrifuge speed and time can be adjusted to obtain better extraction).
   2.3.8.1 It will form two liquid layers (phase separation) and pellet of solid in the bottom of the tube.
   2.3.8.2 The bottom liquid layer (DCM rich layer) should have majority of orange color from L and Z.
2.3.9 Prepare 900 μL of acetone in HPLC vials
2.3.10 Take 100 μL of the bottom liquid layer from the centrifuged mixture sample using a pipet carefully not to interrupt the bottom solid pellet and top aqueous layer
2.3.11 Add it into the prepared 900 μL acetone in HPLC vial (1/10× dilution, Dilution factor 10). Be careful not to lose the solution by dripping from the pipet tip, due to the low surface tension of DCM rich solution
2.3.12 Cap the 1/10× diluted extract
2.3.13 Vortex the vial
2.3.14 Inject into HPLC following the HPLC method defined in the later section
2.3.15 At least 3 sample replicates and at least 3 neat condition controls (extracted the same way but matrix (capsule content suspension) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Lutein and Zeaxanthin powder
2.4.3 Suspend the powder in DI water to make a suspension at 1.333 mg/mL for Lutein (equivalent to 1.0 mg/mL of pure Lutein) and 1.429 mg/mL for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: DI water 1:1 mixture (at least 40 mL)
2.4.7 Prepare 10 empty 2 mL Eppendorf tubes for each Lutein and Zeaxanthin
2.4.8 Fill 1 mL of EtOH: DI water mixture into 9 of them (1 empty and 9 with 1 mL EtOH)
2.4.9 Vortex the Lutein stock suspension (or Zeaxanthin stock suspension) and immediately after the agitation, transfer 1.0 mL of the suspension into one empty tube and one of 9 tubes with 1.0 mL EtOH: DI water mixture (making 1 mL as the same concentration and 2 mL of 1/2× dilution)
2.4.10 After agitation, take out 1 mL from 1/2× dilution and add into the next 1 mL pure EtOH: DI water mixture making second 1/2× dilution. Continue this 1/2×serial dilutions into the next tube to make 10 concentration levels for each Lutein and Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L and Z into the DCM rich bottom layer, if necessary)
2.4.13 Centrifuge the tube at 10000 RPM for 2 min (the centrifuge speed and time can be adjusted to obtain better extraction).
   2.4.13.1 It will form two liquid layers (phase separation) and pellet of solid in the bottom of the tube.
   2.4.13.2 The bottom liquid layer (DCM rich layer) should have majority of orange color from L and Z.
2.4.14 Prepare 900 μL of acetone in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL acetone in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in DI water (freshly prepared*, |
| | vortexed, equivalent to 1.0 mg/mL pure lutein) |
| | Zeaxanthin at 1.429 mg/mL in DI water (freshly prepared*, |
| | vortexed, equivalent to 1.0 mg/mL pure zeaxanthin) |
| | L and Z are not soluble and suspendable in water. So, serial |
| | dilution to prepare for the calibration standards should be |
| | performed after adding EtOH (sample:EtOH 1:1) and |
| | diluted into water:EtOH (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.25 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 μg/mL Zeaxanthin |
| | 0.195 ug/mL Zeaxanthin |
| Quality Control Samples for | N/A, to be included in the future |
| Lutein and/or Zeaxanthin, | |
| diluted from PS solutions | |

*All samples must be prepared freshly, due to instability.
** The L and Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution in | | Std conc. |
| solution ID | (μg/mL) | (μL) | (μL) | (μL) ^{b} | Stock ID | (uL) | Acetone | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

### 2.5 Equipments and Supplies

2.5.1 HPLC with UV/VIS detector
2.5.2 YMC C30 carotenoid column, 5 μm, 250×4.6 mm, Part #: CT99S05-2546WT
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic): Pure acetone as stated in 2.1.5
3.1.3 Diluent Blank: Pure acetone as stated in 2.1.5

### 3.2 HPLC Conditions

LC system: Agilent 1260 or 1290, or equivalent
Column: YMC C30 Carotenoid, 5 μm, 250×4.6 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: 100% acetone
Gradient: Not necessary (Isocratic mode)

| Step | Time (min) | A (%) | B (%) |
|---|---|---|---|
| 0 | 0 | 1 | 99* |
| 1 | 5 | 1 | 99* |

*A:B ratio can be changed depending on needs, including but not limited to, 2:98, 3:97, 4:96, 5:95, 6:94, 7:93, 8:92, 9:91, 10:90, 11:89, etc. Please note that as the A portion increases, the retention time for Lutein and Zeaxanthin peaks are delayed, making the run time longer. Also, increase of A portion can separate the Lutein peak and Zeaxanthin peak farther apart.

Mobile phase flow rate: 1.0 mL/min
Injection volume: 20 μL
Run time: 5 minutes
Retention times: Lutein at 4.0 minute/Zeaxanthin at 4.5 minute
Needle Washing Solvent: TBD (tentatively, Acetone or water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |
| L calibration Std 1 to Std 10 | 1 |
| Diluent Blank | 1 |
| Z calibration Std 1 to Std 10 | 1 |
| Diluent Blank | 1 |
| Unknown samples (Lutein/Zeaxanthin/MPs extract | 1 |
| from capsule) | |
| Diluent Blank | 1 |
| L calibration Std 1 to Std 9 | 1 |
| Diluent Blank | 1 |
| Z calibration Std 1 to Std 9 | 1 |
| Diluent Blank | 1 |
| System Suitability (100 μg/mL Lutein) | 3 |
| Diluent Blank | 1 |
| System Suitability (100 μg/mL Zeaxanthin) | 3 |
| Diluent Blank | 1 |

### 3.4 Data Analysis

Note: Refer to Attachment 5.1.1 for example integrated chromatograms and recovery.
3.4.1 System Suitability
   3.4.1.1 The chromatographic profile of the 100 μg/mL L and Z Standards (or any other concentrations chosen as quality control purpose, determined using fresh L and Z) are comparable to the chromatogram determined using very fresh L and Z.
   3.4.1.2% RSD≤3 for the main peak areas of the six 100 μg/mL L and Z Standard (or any other concentrations chosen as quality control purpose, determined using fresh L and Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L and Z standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L and Z, calculate the concentration of L and Z using the following equation:

$$L or Z ( \frac{mg}{mL} ) = \frac{{A}_{Test} - y intercept}{slope} \times DF \times 0.001$$

Aₜₑₛₜ=Average main peak area of either L or Z
DF=dilution factor of the test sample.
0.001=convert μg/mL to mg/mL
3.4.3.2 Calculate the percent recovery of the test sample as:

$$\frac{Extracted L or Z concentration ( \frac{mg}{mL} )}{Neat condition control L test sample ( \frac{mg}{mL} )} \times 100$$

3.4.3.3 Calculate the percent amount of L and Z loading as:

$$\frac{Determined sample L or Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L/Z in the test sample to one decimal place (e.g. 96.5%).
3.5.2 Report the percent free L/Z in the test sample to one decimal place (e.g.
3.5%).
   4.0

### 5.0 Attachments

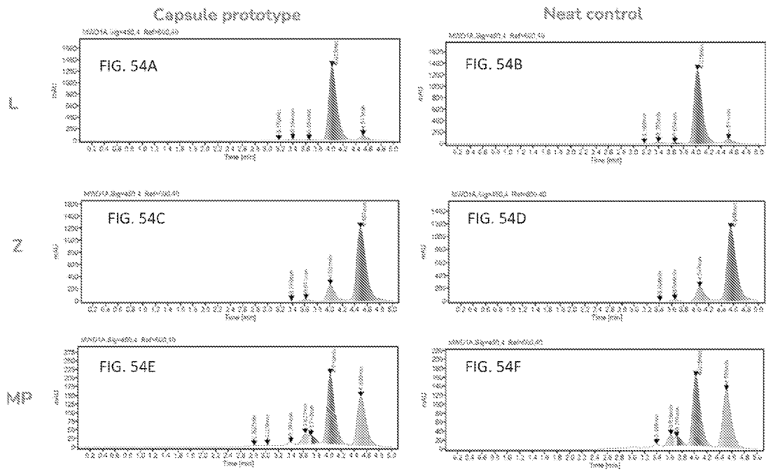

5.1.1 Preliminary extraction results and recovery are presented in FIG. 54A-FIG. 54F and summarized in the table below.

| | AUC | AUC | Recovery |
|---|---|---|---|
| Capsule prototype | (Capsule) | (Neat) | (%) |
| Lutein | 11453.3 | 11845.2 | 96.7 |
| Zeaxanthin | 12770.6 | 12035.5 | 106.1 |
| P81 (L) | 1466.6 | 1461.6 | 100.3 |
| P81 (Z) | 1298.15 | 1318.3 | 98.5 |
| Prototype only avg | 294.9 (L) | 63.45 (Z) | |

Due to unavailable appropriate blank for capsule contents, 2 mg/mL of prototype capsule was used as a blank and L/Z were spiked into it. The values of prototype blank were subtracted from spiked L/Z values for calculation

### Appendix E: Quantification Method for Encapsulated Lutein Levels in Formulations by HPLC

### 1.0 Purpose

The purpose of this Standard Analytical Method is to establish the sample preparation of Lutein encapsulated microparticles, and associated analytical methods for the quantitative determination of encapsulated and free Lutein by HPLC

### 2.0 Materials and Solutions

### 2.1 Solutions

2.1.1 Dichloromethane (DCM), ACS reagent, reag. ISO, ≥99.9% (GC): Sigma-Aldrich, Part #: 32222, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent
2.1.6 Acetonitrile with 0.1% formic acid, hypergrade for LC-MS LiChrosolv®: Sigma-Aldrich, Part #: 1590021000, or equivalent

### 2.2 Reference Materials

| Name | Resource, Purity, and Others (Expiration Date) |
|---|---|
| Lutein (powder)* | CHENGUANG BIOTECH GROUP CO., LTD, |
| | or IOSA |
| | 75% Lutein, Exp Date: 2 years from |
| | manufacturing date (unknown) |
| Lutein microparticles | Southwest Research Institute, Lutein loading (%) |
| (powder)** | and other additives (%), Exp Date: Unknown |

*Once the manufacturer packages are opened, aliquot them in small quantity for a single use or short term use only (a few 100 s mgs in glass vials), N₂ gas head them, then stored them in −80° C. freezer until use. When used, they should be equilibrated to room temperature at least for 30 minutes before opening aliquots.

### 2.3 Primary Stock Solutions, Spiking Stock Solutions, Calibration Standards/QC Samples

| Solutions | Concentration (range) |
|---|---|
| Primary Stock Solutions (PS) | Lutein at 1.333 mg/mL in DCM (freshly prepared*, |
| | vortexed, equivalent to 1.0 mg/mL pure lutein) |
| | Lutein microparticles at 1 mg/mL in DCM (freshly |
| | prepared*, vortexed) |
| Internal Standard Solution (IS) | N/A |
| Calibration Standard Samples | 100 μg/mL Lutein in DCM |
| for Lutein, diluted from PS | 50 μg/mL Lutein in DCM |
| solutions | 25 μg/mL Lutein in DCM |
| | 12.5 μg/mL Lutein in DCM |
| | 6.25 μg/mL Lutein in DCM |
| | 3.125 μg/mL Lutein in DCM |
| | 1.563 μg/mL Lutein in DCM |
| | 0.781 μg/mL Lutein in DCM |
| | 0.391 ug · mL Lutein in DCM |
| Quality Control Samples for | N/A, to be included in the future |
| Lutein, diluted from PS | |
| solutions | |

*All samples must be prepared freshly, due to instability.
** The L range are subject to be changed depending on the instrument's sensitivity and maximum detection range.

Below is an example of how to prepare calibration standards from stock solutions

| | | Vol. | Vol. | | | |
|---|---|---|---|---|---|---|
| | Spiking | Spiking | DCM | | | |
| | Solution | solution | (or | Total | | |
| Spiking | Conc. | to add | dilulent) | Volume | Conc. | STD/QC |
| solution ID | (μg/mL) | (μL) | (μL) | (μL) ^{b} | (μg/mL) | ID |
| Lutein stock | 1000 (1333)* | 200 | 1800 | 1000 | 100 | L STD 9 |
| L STD9 | 100 | 1000 | 1000 | | 50 | L STD 8 |
| L STD8 | 50 | 1000 | 1000 | | 25 | L STD 7 |
| L STD 7 | 25 | 1000 | 1000 | | 12.5 | L STD 6 |
| L STD 6 | 12.5 | 1000 | 1000 | | 6.25 | L STD 5 |
| L STD 5 | 6.25 | 1000 | 1000 | | 3.125 | L STD 4 |
| L STD 4 | 3.125 | 1000 | 1000 | | 1.563 | L STD 3 |
| L STD 3 | 1.563 | 1000 | 1000 | | 0.781 | L STD 2 |
| L STD 2 | 0.781 | 1000 | 1000 | | 0.391 | L STD 1 |
| | | | 2000 | | 0 | Blank |

*Lutein PS concentration including impurity

### 2.4 Equipments and Supplies

2.4.1 HPLC with UV/VIS detector
2.4.2 Phenomenex Gemini® C18 column, 3 μm, 100×3 mm, Part #: 00D-4439Y0, or any equivalent C18 columns
2.4.3 Borosilicate glass bottles: 500 mL, 1 L, or 2 L capacity
2.4.4 Borosilicate glass vials, 4 mL, 7 mL, and 20 mL capacity
2.4.5 Positive displacement pipettes capable of measuring volumes between 20 μL to 25 mL
2.4.6 2 mL glass HPLC vials with PTFE/Silicone septa vial caps
2.4.7 Analytical balance
2.4.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Lutein at 1.95 minute
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| L Linearity Std 1 to Std 9 | 1 |
| Diluent Blank | 1 |
| Unknown samples (Lutein/MPs) | 1 |
| Diluent Blank | 1 |
| L Linearity Std 1 to Std 9 | 1 |
| Diluent Blank | 1 |
| System Suitability (100 μg/mL Lutein) | 3 |
| Diluent Blank | 1 |

### 3.4 Data Analysis

Note: Refer to Attachment 5.1.1 for example integrated chromatograms.
3.4.1 System Suitability
   3.4.1.1 The chromatographic profile of the 100 μg/mL L Standards (or any other concentrations chosen as quality control purpose, determined using fresh L) are comparable to the chromatogram determined using very fresh L.
   3.4.1.2% RSD≤3 for the main peak areas of the six 100 μg/mL L Standard (or any other concentrations chosen as quality control purpose, determined using fresh L) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²≥0.99.
   3.4.2.5 The percent recovery of standard levels must be within 90% to 110% of its label claim.
3.4.3 Lutein Concentrations in samples
   3.4.3.1 Using the standard curve values for L, calculate the concentration of L using the following equation:

$$L ( \frac{mg}{mL} ) = \frac{{A}_{Test} - y intercept}{slope} \times DF \times 0.001$$

Aₜₑₛₜ=Average main peak area of L
DF=dilution factor of the test sample.
0.001=convert μg/mL to mg/mL
3.4.3.2 Calculate the percent recovery of the test sample as:

$$\frac{Sample L concentration ( \frac{mg}{mL} )}{Label Claim of L test sample ( \frac{mg}{mL} )} \times 100$$

3.4.3.3 Calculate the percent amount of L loading as:

$$\frac{Sample L concentration ( \frac{mg}{mL} )}{MP concentration ( \frac{mg}{mL} )} \times 100$$

3.4.3.4 The percent recovery of each test sample must be within 85% to 110% of its label claim.

### 3.5 Reporting

3.5.1 Report the percent encapsulated L in the test sample to one decimal place (e.g. 96.5%).
3.5.2 Report the percent free L in the test sample to one decimal place (e.g. 3.5%).
   4.0

### 5.0 Attachments

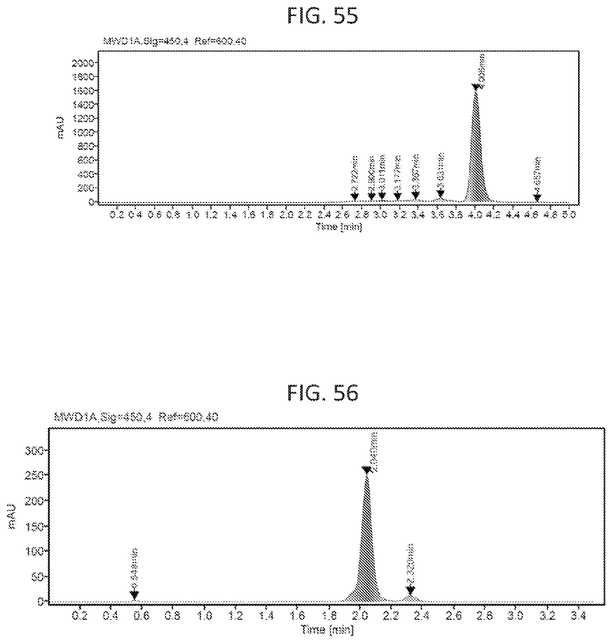

5.1.1 An example of a chromatograms is presented in FIG. 55, which presents Lutein at a concentration of 100 μg/mL in DCM having a main peak at 1.95 minute retention time.

### Appendix F: Quantification Method for Lutein Levels from Powder Milk Solution by HPLC

### 1.0 Purpose

The purpose of this Standard Operating Procedure is to establish the sample preparation of Lutein encapsulated microparticles, extracted from powder milk solution and associated analytical methods for the quantitative determination of Lutein by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent

### 2.2 Reference Materials

| Name | Resource, Purity, and Others (Expiration Date) |
|---|---|
| Lutein (powder)* | CHENGUANG BIOTECH GROUP CO., LTD, or |
| | IOSA |
| | 75% Lutein, Exp Date: 2 years from manufacturing |
| | date (unknown) |
| Lutein | Southwest Research Institute, Lutein loading (%) |
| microparticles | and other ingredients (%), Exp Date: TBD |
| (powder)** | |
| Powder milk | Fonterra, High fat milk powder from whole milk, |
| | Lot: 23137233, Stock ID: 659 or equivalent |

*Once the manufacturer packages of Lutein and Zeaxanthin are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein extraction from powder milk by liquid-liquid extraction method
2.3.1 Weigh powder milk (e.g. 2 g)
2.3.2 Add certain amount of DI water to make 10% (or any other concentrations depending on needs) (e.g. 2 g powder milk in 18 mL water to make about 20 mL 10% powder milk solution) and use this solution as matrix stock solution
2.3.3 Weight L encapsulated microparticles
2.3.4 Add powder milk solution to the L microparticles to make the concentration of interest
2.3.5 Vortex and sonicate the powder milk solutions containing L microparticles
2.3.6 Immediately after the agitation, transfer 0.5 mL of the powder milk solution into 2 mL Eppendorf vial
2.3.7 Add 0.5 mL of Ethanol into the powder milk solution to make 1 mL volume (the volume can be increased to make better dissolution if necessary) and vortex to cause partial dissolution of L microparticles and to induce release of L from the powder milk contents and MP matrix.
2.3.8 Add 0.5 mL of dichloromethane into the suspension and vortex (the volume can be increased to make better dissolution and better separation of L into the DCM rich bottom layer, if necessary)
2.3.9 Centrifuge the tube at 10000 RPM for 2 min (the centrifuge speed and time can be adjusted to obtain better extraction).
   2.3.9.1 It will form three layers, two liquid layers (phase separation) and a solid layer of powder milk contents in between those two liquid layers.
   2.3.9.2 The bottom liquid layer (DCM rich layer) should have majority of orange color from L.
2.3.10 Prepare 900 μL of DCM in HPLC vials
2.3.11 Take 100 μL of the bottom liquid layer from the centrifuged mixture sample using a pipet carefully not to interrupt the bottom solid pellet and top aqueous layer
2.3.12 Add it into the prepared 900 μL DCM in HPLC vial (1/10× dilution, Dilution factor 10). Be careful not to lose the solution by dripping from the pipet tip, due to the low surface tension of DCM rich solution
2.3.13 Cap the 1/10× diluted extract
2.3.14 Vortex the vial
2.3.15 Inject into HPLC following the HPLC method defined in the later section
2.3.16 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Lutein powder
2.4.3 Suspend the powder in the powder milk to make a suspension at 1.333 mg/mL for Lutein (equivalent to 1.0 mg/mL of pure Lutein) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: powder milk 1:1 mixture (at least 40 mL)
2.4.7 Prepare 10 empty 2 mL Eppendorf tubes for each Lutein
2.4.8 Fill 1 mL of EtOH: powder milk mixture into 9 of them (1 empty and 9 with 1 mL EtOH)
2.4.9 Vortex the Lutein stock suspension and immediately after the agitation, transfer 1.0 mL of the suspension into one empty tube and one of 9 tubes with
1.0 mL EtOH: powder milk mixture (making 1 mL as the same concentration and 2 mL of 1/2× dilution)
2.4.10 After agitation, take out 1 mL from 1/2× dilution and add into the next 1 mL pure EtOH: powder milk mixture making second 1/2× dilution. Continue this
1/2× serial dilutions into the next tube to make 10 concentration levels for each Lutein
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of powder milk contents in between those two liquid layers.
   2.4.13.2 The bottom liquid layer (DCM rich layer) should have majority of orange color from L and Z.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in pure milk (freshly prepared*, |
| | vortexed, equivalent to 1.0 mg/mL pure lutein) |
| | L is not soluble and suspendable in milk. So, serial dilution |
| | to prepare for the calibration standards should be performed |
| | after adding EtOH (powder milk sample:EtOH 1:1) and |
| | diluted into EtOH:powder milk (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Quality Control Samples for | N/A, to be included in the future |
| Lutein and/or Zeaxanthin, | |
| diluted from PS solutions | |

*All samples must be prepared freshly, due to instability.
** The L and Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:powder | Total | | DCM | | | Nominal |
| Spiking | Conc. | to add | milk mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

### 2.5 Equipments and Supplies

2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Lutein at 1.95 minute
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| L calibration Std 1 to Std 10 | 1 |
| Diluent Blank | 1 |
| Unknown samples (Lutein/MPs extract from milk) | 1 |
| Diluent Blank | 1 |
| L calibration Std 1 to Std 9 | 1 |
| Diluent Blank | 1 |
| System Suitability (100 μg/mL Lutein) | 3 |
| Diluent Blank | 1 |

### 3.4 Data Analysis

3.4.1 System Suitability
   3.4.1.1 The chromatographic profile of the 100 μg/mL L Standards (or any other concentrations chosen as quality control purpose, determined using fresh L) are comparable to the chromatogram determined using very fresh L.
   3.4.1.2% RSD≤3 for the main peak areas of the six 100 μg/mL L Standard (or any other concentrations chosen as quality control purpose, determined using fresh L) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L, calculate the concentration of L using the following equation:

$$L ( \frac{mg}{mL} ) = \frac{{A}_{Test} - y intercept}{slope} \times DF \times 0.001$$

Aₜₑₛₜ=Average main peak area of L
DF=dilution factor of the test sample.
0.001=convert μg/mL to mg/mL
3.4.3.2 Calculate the percent recovery of the test sample as:

$$\frac{Extracted L concentration ( \frac{mg}{mL} )}{Neat condition control L test sample ( \frac{mg}{mL} )} \times 100$$

3.4.3.3 Calculate the percent amount of L loading as:

$$\frac{Determined sample L concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L in the test sample to one decimal place (e.g. 96.5%).
3.5.2 Report the percent free L in the test sample to one decimal place (e.g. 3.5%).
   4.0

### 5.0 Attachments

5.1.1 Assay worksheet
   Attachment 5.1.1

### Appendix G: Quantification Method for Lutein Levels from Milk by HPLC

### 1.0 Purpose

The purpose of this Standard Operating Procedure is to establish the sample preparation of Lutein encapsulated microparticles, extracted from milk and associated analytical methods for the quantitative determination of Lutein by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent
2.1.7 Whole milk or Lutein (free or microparticles) fortified milk

### 2.2 Reference Materials

| Name | Resource, Purity, and Others (Expiration Date) |
|---|---|
| Lutein (powder)* | CHENGUANG BIOTECH GROUP CO., LTD, or |
| | IOSA |
| | 75% Lutein, Exp Date: 2 years from manufacturing |
| | date (unknown) |
| Lutein | Southwest Research Institute, Lutein loading (%) |
| microparticles | and other ingredients (%), Exp Date: TBD |
| (powder)** | |

*Once the manufacturer packages are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein extraction from milk by liquid-liquid extraction method
2.3.1 For regular milk, weigh certain mg of L encapsulated microparticles and spike them into the milk
2.3.2 For L microparticle fortified milk product, use the milk product directly
2.3.3 Vortex and sonicate the milk solutions containing L microparticles
2.3.4 Immediately after the agitation, transfer 0.5 mL of the milk solution into 2 mL Eppendorf vial
2.3.5 Add 0.5 mL of Ethanol into the milk solution to make 1 mL volume (the volume can be increased to make better dissolution if necessary) and vortex to cause partial dissolution of L microparticles and to induce release of L/Z from milk contents and MP matrix.
2.3.6 Add 0.5 mL of dichloromethane into the suspension and vortex (the volume can be increased to make better dissolution and better separation of L into the DCM rich bottom layer, if necessary)
2.3.7 Centrifuge the tube at 10000 RPM for 2 min (the centrifuge speed and time can be adjusted to obtain better extraction).
   2.3.7.1 It will form three layers, two liquid layers (phase separation) and a solid layer of milk contents in between those two liquid layers.
   2.3.7.2 The bottom liquid layer (DCM rich layer) should have majority of orange color from L.
2.3.8 Prepare 900 μL of DCM in HPLC vials
2.3.9 Take 100 μL of the bottom liquid layer from the centrifuged mixture sample using a pipet carefully not to interrupt the bottom solid pellet and top aqueous layer
2.3.10 Add it into the prepared 900 μL DCM in HPLC vial (1/10× dilution, Dilution factor 10). Be careful not to lose the solution by dripping from the pipet tip, due to the low surface tension of DCM rich solution
2.3.11 Cap the 1/10× diluted extract
2.3.12 Vortex the vial
2.3.13 Inject into HPLC following the HPLC method defined in the later section
2.3.14 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Lutein powder
2.4.3 Suspend the powder in pure milk to make a suspension at 1.333 mg/mL for Lutein (equivalent to 1.0 mg/mL of pure Lutein) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: pure milk 1:1 mixture (at least 40 mL)
2.4.7 Prepare 10 empty 2 mL Eppendorf tubes for each Lutein
2.4.8 Fill 1 mL of EtOH: pure milk mixture into 9 of them (1 empty and 9 with 1 mL EtOH)
2.4.9 Vortex the Lutein stock suspension and immediately after the agitation, transfer 1.0 mL of the suspension into one empty tube and one of 9 tubes with
1.0 mL EtOH: pure milk mixture (making 1 mL as the same concentration and
2 mL of 1/2× dilution)
2.4.10 After agitation, take out 1 mL from 1/2× dilution and add into the next 1 mL pure EtOH: pure milk mixture making second 1/2× dilution. Continue this 1/2×serial dilutions into the next tube to make 10 concentration levels for each Lutein
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of milk contents in between those two liquid layers.
   2.4.13.2 The bottom liquid layer (DCM rich layer) should have majority of orange color from L.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in pure milk (freshly prepared*, |
| | vortexed, equivalent to 1.0 mg/mL pure lutein) |
| | L is not soluble and suspendable in milk. So, serial dilution |
| | to prepare for the calibration standards should be performed |
| | after adding EtOH (milk sample:EtOH 1:1) and diluted into |
| | EtOH:pure milk (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Quality Control Samples for | N/A, to be included in the future |
| Lutein diluted from PS | |
| solutions | |

*All samples must be prepared freshly, due to instability.
** The L range are subject to be changed depending on the instrument's sensitivity and maximum detection range.

Below is an example of how to prepare calibration standards from stock solutions

| | | Vol. | Vol. | | | | | | |
|---|---|---|---|---|---|---|---|---|---|
| | Spiking | Spiking | EtOH:pure | | | Vol. | | | |
| | Solution | solution | milk | Total | | DCM | | | Nominal |
| Spiking | Conc. | to add | mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

### 2.5 Equipments and Supplies

2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Lutein at 1.95
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| L calibration Std 1 to Std 10 | 1 |
| Diluent Blank | 1 |
| Unknown samples (Lutein/Zeaxanthin/MPs extract | 1 |
| from milk) | |
| Diluent Blank | 1 |
| L calibration Std 1 to Std 9 | 1 |
| Diluent Blank | 1 |
| System Suitability (100 μg/mL Lutein) | 3 |
| Diluent Blank | 1 |

### 3.4 Data Analysis

3.4.1 System Suitability
   3.4.1.1 The chromatographic profile of the 100 μg/mL L Standards (or any other concentrations chosen as quality control purpose, determined using fresh L) are comparable to the chromatogram determined using very fresh L.
   3.4.1.2% RSD≤3 for the main peak areas of the six 100 μg/mL L Standard (or any other concentrations chosen as quality control purpose, determined using fresh L) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L, calculate the concentration of L using the following equation:

$$L ( \frac{mg}{mL} ) = \frac{{A}_{Test} - y intercept}{slope} \times DF \times 0.001$$

Aₜₑₛₜ=Average main peak area of L
DF=dilution factor of the test sample.
0.001=convert μg/mL to mg/mL
3.4.3.2 Calculate the percent recovery of the test sample as:

$$\frac{Extracted L concentration ( \frac{mg}{mL} )}{Neat condition control L test sample ( \frac{mg}{mL} )} \times 100$$

3.4.3.3 Calculate the percent amount of L loading as:

$$\frac{Determined sample L concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L in the test sample to one decimal place (e.g. 96.5%).
3.5.2 Report the percent free L in the test sample to one decimal place (e.g. 3.5%).

### Appendix H: Quantification Method for Lutein Levels from Probiotic Capsules by HPLC

### 1.0 Purpose

The purpose of this Standard Operating Procedure is to establish the sample preparation of Lutein encapsulated microparticles, extracted from probiotic capsule product with other ingredients and associated analytical methods for the quantitative determination of Lutein by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent

### 2.2 Reference Materials

| Name | Resource, Purity, and Others (Expiration Date) |
|---|---|
| Lutein (powder)* | CHENGUANG BIOTECH GROUP CO., LTD, or |
| | IOSA |
| | 75% Lutein, Exp Date: 2 years from manufacturing |
| | date (unknown) |
| Lutein | Southwest Research Institute, Lutein loading ( %) |
| microparticles | and other ingredients (%), Exp Date: TBD |
| (powder)** | |
| Probiotics Capsule | Manufacturer: TBD |
| product | Lutein content (% or mg) Zeaxanthin content (% |
| containing L/Z | or mg), and other ingredients (% or mg), Exp |
| microparticles | Date: TBD |

*Once the manufacturer packages are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Lutein extraction from probiotics capsule content mixture by liquid-liquid extraction method
2.3.1 Open capsule and collect powder contents in a separate vial
2.3.2 Weigh certain mg of the collected capsule content powder
2.3.3 Suspend the powder in DI water to make a suspension at 20 mg/mL concentration
2.3.4 Vortex and sonicate the suspension
2.3.5 Immediately after the agitation, transfer 0.5 mL of the suspension into 2 mL Eppendorf vial
2.3.6 Add 0.5 mL of Ethanol into the suspension and vortex to form partially dissolved suspension to make 1 mL volume (the volume can be increased to make better dissolution if necessary)
2.3.7 Add 0.5 mL of dichloromethane into the suspension and vortex (the volume can be increased to make better dissolution and better separation of L into the DCM rich bottom layer, if necessary)
2.3.8 Centrifuge the tube at 10000 RPM for 2 min (the centrifuge speed and time can be adjusted to obtain better extraction).
   2.3.8.1 It will form two liquid layers (phase separation) and pellet of solid in the bottom of the tube.
   2.3.8.2 The bottom liquid layer (DCM rich layer) should have majority of orange color from L and Z.
2.3.9 Prepare 900 μL of DCM in HPLC vials
2.3.10 Take 100 μL of the bottom liquid layer from the centrifuged mixture sample using a pipet carefully not to interrupt the bottom solid pellet and top aqueous layer
2.3.11 Add it into the prepared 900 μL DCM in HPLC vial (1/10× dilution, Dilution factor 10). Be careful not to lose the solution by dripping from the pipet tip, due to the low surface tension of DCM rich solution
2.3.12 Cap the 1/10× diluted extract
2.3.13 Vortex the vial
2.3.14 Inject into HPLC following the HPLC method defined in the later section
2.3.15 At least 3 sample replicates and at least 3 neat condition controls (extracted the same way but matrix (capsule content suspension) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Lutein powder
2.4.3 Suspend the powder in DI water to make a suspension at 1.333 mg/mL for Lutein (equivalent to 1.0 mg/mL of pure Lutein) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: DI water 1:1 mixture (at least 40 mL)
2.4.7 Prepare 10 empty 2 mL Eppendorf tubes for each
2.4.8 Fill 1 mL of EtOH: DI water mixture into 9 of them (1 empty and 9 with 1 mL EtOH)
2.4.9 Vortex the Lutein stock suspension and immediately after the agitation, transfer 1.0 mL of the suspension into one empty tube and one of 9 tubes with
1.0 mL EtOH: DI water mixture (making 1 mL as the same concentration and
2 mL of 1/2× dilution)
2.4.10 After agitation, take out 1 mL from 1/2× dilution and add into the next 1 mL pure EtOH: DI water mixture making second 1/2× dilution. Continue this 1/2×serial dilutions into the next tube to make 10 concentration levels for each Lutein
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L into the DCM rich bottom layer, if necessary)
2.4.13 Centrifuge the tube at 10000 RPM for 2 min (the centrifuge speed and time can be adjusted to obtain better extraction).
   2.4.13.1 It will form two liquid layers (phase separation) and pellet of solid in the bottom of the tube.
   2.4.13.2 The bottom liquid layer (DCM rich layer) should have majority of orange color from L.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Lutein at 1.333 mg/mL in DI water (freshly prepared*, |
| | vortexed, equivalent to 1.0 mg/mL pure lutein) |
| | L is not soluble and suspendable in water. So, serial dilution |
| | to prepare for the calibration standards should be performed |
| | after adding EtOH (sample:EtOH 1:1) and diluted into |
| | water:EtOH (1:1) mixture |
| Internal Standard Solution (IS) | N/A |
| Calibration Standard Samples | 100 μg/mL Lutein |
| for Lutein, diluted from PS | 50 μg/mL Lutein |
| solutions (Note: they are | 25 μg/mL Lutein |
| nominal concentration by input | 12.5 μg/mL Lutein |
| without considering dilutions | 6.2 μg/mL Lutein |
| during extraction process, not | 3.125 μg/mL Lutein |
| actual concentration) ** | 1.563 μg/mL Lutein |
| | 0.781 μg/mL Lutein |
| | 0.391 ug/mL Lutein |
| | 0.195 ug/mL Lutein |
| Quality Control Samples for | N/A, to be included in the future |
| Lutein and/or Zeaxanthin, | |
| diluted from PS solutions | |

*All samples must be prepared freshly, due to instability.
** The L range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Lutein | 1000 (1333)* | | | 1000 | L stock 10 | 500 | 1/10x | L STD 10 | 100 |
| Primary | | | | | | | | | |
| stock | | | | | | | | | |
| L Stock 10 | 1000 | 1000 | 1000 | | L Stock 9 | 500 | 1/10x | L STD 9 | 50 |
| L Stock 9 | 500 | 1000 | 1000 | | L Stock 8 | 500 | 1/10x | L STD 8 | 25 |
| L Stock 8 | 250 | 1000 | 1000 | | L Stock 7 | 500 | 1/10x | L STD 7 | 12.5 |
| L Stock 7 | 125 | 1000 | 1000 | | L Stock 6 | 500 | 1/10x | L STD 6 | 6.25 |
| L Stock 6 | 62.5 | 1000 | 1000 | | L Stock 5 | 500 | 1/10x | L STD 5 | 3.125 |
| L Stock 5 | 31.25 | 1000 | 1000 | | L Stock 4 | 500 | 1/10x | L STD 4 | 1.563 |
| L Stock 4 | 15.63 | 1000 | 1000 | | L Stock 3 | 500 | 1/10x | L STD 3 | 0.781 |
| L Stock 3 | 7.81 | 1000 | 1000 | | L Stock 2 | 500 | 1/10x | L STD 2 | 0.391 |
| L Stock 2 | 3.91 | 1000 | 1000 | | L Stock 1 | 500 | 1/10x | L STD 1 | 0.195 |
| | | | 1000 | | Blank | 500 | 1/10x | | |

*Lutein PS concentration including impurity
**These concentrations are nominal concentrations not actual concentration

### 2.5 Equipments and Supplies

2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Lutein at 1.95 minute
Needle Washing Solvent: TBD (tentatively, Water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Lutein) | 3 |
| Diluent Blank | 1 |
| L calibration Std 1 to Std 10 | 1 |
| Diluent Blank | 1 |
| Unknown samples (Lutein/MPs extract from | 1 |
| capsule) | |
| Diluent Blank | 1 |
| L calibration Std 1 to Std 9 | 1 |
| Diluent Blank | 1 |
| System Suitability (100 μg/mL Lutein) | 3 |
| Diluent Blank | 1 |

### 3.4 Data Analysis

System Suitability
   3.4.1.1 The chromatographic profile of the 100 μg/mL L Standards (or any other concentrations chosen as quality control purpose, determined using fresh L) are comparable to the chromatogram determined using very fresh L.
   3.4.1.2% RSD≤3 for the main peak areas of the six 100 μg/mL L Standard (or any other concentrations chosen as quality control purpose, determined using fresh L) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Lutein, Zeaxanthin Concentrations in samples
   3.4.3.1 Using the standard curve values for L, calculate the concentration of L using the following equation:

$$L ( \frac{mg}{mL} ) = \frac{{A}_{Test} - y intercept}{slope} \times DF \times 0.001$$

Aₜₑₛₜ=Average main peak area of L
DF=dilution factor of the test sample.
0.001=convert μg/mL to mg/mL
3.4.3.2 Calculate the percent recovery of the test sample as:

$$\frac{Extracted L concentration ( \frac{mg}{mL} )}{Neat condition control L test sample ( \frac{mg}{mL} )} \times 100$$

3.4.3.3 Calculate the percent amount of L loading as:

$$\frac{Determined sample L concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated L in the test sample to one decimal place (e.g. 96.5%).
3.5.2 Report the percent free L in the test sample to one decimal place (e.g. 3.5%).

### Appendix I: Quantification Method for Encapsulated Zeaxanthin Levels in Formulation by HPLC

### 1.0 Purpose

The purpose of this Standard Analytical Method is to establish the sample preparation of Zeaxanthin encapsulated microparticles, and associated analytical methods for the quantitative determination of encapsulated and free Zeaxanthin by HPLC

### 2.0 Materials and Solutions

### 2.1 Solutions

2.1.1 Dichloromethane (DCM), ACS reagent, reag. ISO, ≥99.9% (GC): Sigma-Aldrich, Part #: 32222, or equivalent
2.1.2 Water, HPLC grade: Sigma-Aldrich, Part #: 270733, or equivalent
2.1.3 Formic acid, reagent grade (>95%): Sigma-Aldrich, Part #: F0507, or equivalent or higher quality grade
2.1.4 Water with 0.1% formic acid: Sigma-Aldrich, Part #: 4.85085, or equivalent
2.1.5 Acetonitrile, HPLC grade: Sigma-Aldrich, Part #: 34851, or equivalent
2.1.6 Acetonitrile with 0.1% formic acid, hypergrade for LC-MS LiChrosolv®: Sigma-Aldrich, Part #: 1590021000, or equivalent

### 2.2 Reference Materials

| Name | Resource, Purity, and Others (Expiration Date) |
|---|---|
| Zeaxanthin (powder)* | Shandong Tianyin Biotechnology Co., LTD, or |
| | IOSA |
| | 70% Zeaxanthin, Exp Date: Unknown |
| Zeaxanthin | Southwest Research Institute, Zeaxanthin loading |
| microparticles | (%), and other additives (%), Exp Date: Unknown |
| (powder)** | |

*Once the manufacturer packages are opened, aliquot them in small quantity for a single use or short term use only (a few 100s mgs in glass vials), N₂ gas head them, then stored them in −80° C. freezer until use. When used, they should be equilibrated to room temperature at least for 30 minutes before opening aliquots.

### 2.3 Primary Stock Solutions, Spiking Stock Solutions, Calibration Standards/QC Samples

| Solutions | Concentration (range) |
|---|---|
| Primary Stock Solutions (PS) | Zeaxanthin at 1.429 mg/mL in DCM |
| | (freshly prepared*, vortexed, equivalent |
| | to 1.0 mg/mL pure zeaxanthin) |
| | Zeaxanthin microparticles at 1 mg/mL |
| | in DCM (freshly prepared*, vortexed) |
| Internal Standard Solution (IS) | N/A |
| Calibration Standard | 100 μg/mL Zeaxanthin in DCM |
| Samples for Zeaxanthin, | 50 μg/mL Zeaxanthin in DCM |
| diluted from PS solutions | 25 μg/mL Zeaxanthin in DCM |
| | 12.5 μg/mL Zeaxanthin in DCM |
| | 6.25 μg/mL Zeaxanthin in DCM |
| | 3.125 μg/mL Zeaxanthin in DCM |
| | 1.563 μg/mL Zeaxanthin in DCM |
| | 0.781 μg/mL Zeaxanthin in DCM |
| | 0.391 μg/mL Zeaxanthin in DCM |
| Quality Control Samples | N/A, to be included in the future |
| for Zeaxanthin, diluted | |
| from PS solutions | |

*All samples must be prepared freshly, due to instability.
** The Z range are subject to be changed depending on the instrument's sensitivity and maximum detection range.

Below is an example of how to prepare calibration standards from stock solutions

| | | Vol. | Vol. | | | |
|---|---|---|---|---|---|---|
| | Spiking | Spiking | DCM | | | |
| | Solution | solution | (or | Total | | |
| Spiking | Conc. | to add | dilulent) | Volume | Conc. | STD/QC |
| solution ID | (μg/mL) | (μL) | (μL) | (μL) ^{b} | (μg/mL) | ID |
| Zeaxanthin | 1000 (1429)** | 200 | 1800 | 1000 | 100 | Z STD 9 |
| stock | | | | | | |
| Z STD9 | 100 | 1000 | 1000 | | 50 | Z STD 8 |
| Z STD8 | 50 | 1000 | 1000 | | 25 | Z STD 7 |
| Z STD 7 | 25 | 1000 | 1000 | | 12.5 | Z STD 6 |
| Z STD 6 | 12.5 | 1000 | 1000 | | 6.25 | Z STD 5 |
| Z STD 5 | 6.25 | 1000 | 1000 | | 3.125 | Z STD 4 |
| Z STD 4 | 3.125 | 1000 | 1000 | | 1.563 | Z STD 3 |
| Z STD 3 | 1.563 | 1000 | 1000 | | 0.781 | Z STD 2 |
| Z STD 2 | 0.781 | 1000 | 1000 | | 0.391 | Z STD 1 |
| | | | 2000 | | 0 | Blank |

**Zeaxanthin PS concentration including impurity

### 2.4 Equipments and Supplies

2.4.1 HPLC with UV/VIS detector
2.4.2 Phenomenex Gemini® C18 column, 3 μm, 100×3 mm, Part #: 00D-4439Y0, or any equivalent C18 columns
2.4.3 Borosilicate glass bottles: 500 mL, 1 L, or 2 L capacity
2.4.4 Borosilicate glass vials, 4 mL, 7 mL, and 20 mL capacity
2.4.5 Positive displacement pipettes capable of measuring volumes between 20 μL to 25 mL
2.4.6 2 mL glass HPLC vials with PTFE/Silicone septa vial caps
2.4.7 Analytical balance
2.4.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Zeaxanthin at 2.04 minute
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Zeaxanthin) | 3 |
| Diluent Blank | 1 |
| Z Linearity Std 1 to Std 9 | 1 |
| Diluent Blank | 1 |
| Unknown samples (Zeaxanthin/MPs) | 1 |
| Diluent Blank | 1 |
| Z Linearity Std 1 to Std 9 | 1 |
| Diluent Blank | 1 |
| System Suitability (100 μg/mL Zeaxanthin) | 3 |
| Diluent Blank | 1 |

### 3.4 Data Analysis

Note: Refer to Attachment 5.1.1 for example integrated chromatograms.
3.4.1 System Suitability
   3.4.1.1 The chromatographic profile of the 100 μg/mL Z Standards (or any other concentrations chosen as quality control purpose, determined using fresh Z) are comparable to the chromatogram determined using very fresh Z.
   3.4.1.2% RSD≤3 for the main peak areas of the six 100 μg/mL Z Standard (or any other concentrations chosen as quality control purpose, determined using fresh Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 ForZ standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²≥0.99.
   3.4.2.5 The percent recovery of standard levels must be within 90% to 110% of its label claim.
3.4.3 Zeaxanthin Concentrations in samples
   3.4.3.1 Using the standard curve values for Z, calculate the concentration of Z using the following equation:

$$Z ( \frac{mg}{mL} ) = \frac{{A}_{Test} - y intercept}{slope} \times DF \times 0.001$$

Aₜₑₛₜ=Average main peak area of either Z
DF=dilution factor of the test sample.
0.001=convert μg/mL to mg/mL
3.4.3.2 Calculate the percent recovery of the test sample as:

$$\frac{Sample Z concentration ( \frac{mg}{mL} )}{Label Claim of Z test sample ( \frac{mg}{mL} )} \times 100$$

3.4.3.3 Calculate the percent amount of Z loading as:

$$\frac{Sample Z concentration ( \frac{mg}{mL} )}{MP concentration ( \frac{mg}{mL} )} \times 100$$

3.4.3.4 The percent recovery of each test sample must be within 85% to 110% of its label claim.

### 3.5 Reporting

3.5.1 Report the percent encapsulated Z in the test sample to one decimal place (e.g. 96.5%).
3.5.2 Report the percent free Z in the test sample to one decimal place (e.g. 3.5%).
   4.0

### 5.0 Attachments

5.1.1 Examples of chromatograms are presented in FIG. 56 which presents Zeaxanthin at a concentration of 100 μg/mL in DCM, main peak at 2.04 minute retention time.

### Appendix J: Quantification Method for Zeaxanthin Levels from Powder Milk Solution by HPLC

### 1.0 Purpose

The purpose of this Standard Operating Procedure is to establish the sample preparation of Zeaxanthin encapsulated microparticles, extracted from powder milk solution and associated analytical methods for the quantitative determination of Zeaxanthin by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent

### 2.2 Reference Materials

| Name | Resource, Purity, and Others (Expiration Date) |
|---|---|
| Zeaxanthin | Shandong Tianyin Biotechnology Co., LTD, or IOSA |
| (powder)* | 70% Zeaxanthin, Exp Date: Unknown |
| Zeaxanthin | Southwest Research Institute, Zeaxanthin loading |
| microparticles | (%) and other ingredients (%), Exp Date: TBD |
| (powder)** | |
| Powder milk | Fonterra, High fat milk powder from whole milk, |
| | Lot: 23137233, Stock ID: 659 or equivalent |

*Once the manufacturer packages of Zeaxanthin and Zeaxanthin are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Zeaxanthin extraction from powder milk by liquid-liquid extraction method
2.3.1 Weigh powder milk (e.g. 2 g)
2.3.2 Add certain amount of DI water to make 10% (or any other concentrations depending on needs) (e.g. 2 g powder milk in 18 mL water to make about 20 mL 10% powder milk solution) and use this solution as matrix stock solution
2.3.3 Weight Z encapsulated microparticles
2.3.4 Add powder milk solution to the Z microparticles to make the concentration of interest
2.3.5 Vortex and sonicate the powder milk solutions containing Z microparticles
2.3.6 Immediately after the agitation, transfer 0.5 mL of the powder milk solution into 2 mL Eppendorf vial
2.3.7 Add 0.5 mL of Ethanol into the powder milk solution to make 1 mL volume (the volume can be increased to make better dissolution if necessary) and vortex to cause partial dissolution of Z microparticles and to induce release of L from the powder milk contents and MP matrix.
2.3.8 Add 0.5 mL of dichloromethane into the suspension and vortex (the volume can be increased to make better dissolution and better separation of L into the DCM rich bottom layer, if necessary)
2.3.9 Centrifuge the tube at 10000 RPM for 2 min (the centrifuge speed and time can be adjusted to obtain better extraction).
   2.3.9.1 It will form three layers, two liquid layers (phase separation) and a solid layer of powder milk contents in between those two liquid layers.
   2.3.9.2 The bottom liquid layer (DCM rich layer) should have majority of orange color from Z.
2.3.10 Prepare 900 μL of DCM in HPLC vials
2.3.11 Take 100 μL of the bottom liquid layer from the centrifuged mixture sample using a pipet carefully not to interrupt the bottom solid pellet and top aqueous layer
2.3.12 Add it into the prepared 900 μL DCM in HPLC vial (1/10× dilution, Dilution factor 10). Be careful not to lose the solution by dripping from the pipet tip, due to the low surface tension of DCM rich solution
2.3.13 Cap the 1/10× diluted extract
2.3.14 Vortex the vial
2.3.15 Inject into HPLC following the HPLC method defined in the later section
2.3.16 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Zeaxanthin powder
2.4.3 Suspend the powder in the powder milk to make a suspension at 1.429 mg/ml for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: powder milk 1:1 mixture (at least 40 mL)
2.4.7 Prepare 10 empty 2 mL Eppendorf tubes for each Zeaxanthin
2.4.8 Fill 1 mL of EtOH: powder milk mixture into 9 of them (1 empty and 9 with 1 mL EtOH)
2.4.9 Vortex the Zeaxanthin stock suspension and immediately after the agitation, transfer 1.0 mL of the suspension into one empty tube and one of 9 tubes with
1.0 mL EtOH: powder milk mixture (making 1 mL as the same concentration and 2 mL of 1/2× dilution)
2.4.10 After agitation, take out 1 mL from 1/2× dilution and add into the next 1 mL pure EtOH: powder milk mixture making second 1/2× dilution. Continue this
1/2× serial dilutions into the next tube to make 10 concentration levels for each Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of Z into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of powder milk contents in between those two liquid layers.
   2.4.13.2 The bottom liquid layer (DCM rich layer) should have majority of orange color from Z.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Zeaxanthin at 1.429 mg/mL in pure |
| | milk (freshly prepared*, vortexed, |
| | equivalent to 1.0 mg/mL pure |
| | Zeaxanthin) |
| | Z is not soluble and suspendable in |
| | milk. So, serial dilution to prepare |
| | for the calibration standards should |
| | be performed after adding EtOH |
| | (powder milk sample:EtOH 1:1) and |
| | diluted into EtOH:powder milk (1:1) |
| | mixture |
| Internal Standard Solution (IS) | N/A |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.2 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 ug/mL Zeaxanthin |
| | 0.195 ug/mL Zeaxanthin |
| Quality Control Samples for | N/A, to be included in the future |
| Zeaxanthin and/or Zeaxanthin, | |
| diluted from PS solutions | |

*All samples must be prepared freshly, due to instability.
** The Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

### 2.5 Equipments and Supplies

2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Zeaxanthin at 2.04 minute
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Zeaxanthin) | 3 |
| Diluent Blank | 1 |
| Z calibration Std 1 to Std 10 | 1 |
| Diluent Blank | 1 |
| Unknown samples (Zeaxanthin/MPs extract from milk) | 1 |
| Diluent Blank | 1 |
| Z calibration Std 1 to Std 9 | 1 |
| Diluent Blank | 1 |
| System Suitability (100 μg/mL Zeaxanthin) | 3 |
| Diluent Blank | 1 |

### 3.4 Data Analysis

3.4.1 System Suitability
   3.4.1.1 The chromatographic profile of the 100 μg/mL Z Standards (or any other concentrations chosen as quality control purpose, determined using fresh Z) are comparable to the chromatogram determined using very fresh Z.
   3.4.1.2% RSD≤3 for the main peak areas of the six 100 μg/mL Z Standard (or any other concentrations chosen as quality control purpose, determined using fresh Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²≥0.99.
   3.4.2.5 The percent recovery of standard levels must be within 90% to 110% of its label claim.
3.4.3 Zeaxanthin Concentrations in samples
   3.4.3.1 Using the standard curve values for Z, calculate the concentration of L using the following equation:

$$Z ( \frac{mg}{mL} ) = \frac{{A}_{Test} - y intercept}{slope} \times DF \times 0.001$$

Aₜₑₛₜ=Average main peak area of Z
DF=dilution factor of the test sample.
0.001=convert μg/mL to mg/mL
3.4.3.2 Calculate the percent recovery of the test sample as:

$$\frac{Extracted Z concentration ( \frac{mg}{mL} )}{Neat condition control Z test sample ( \frac{mg}{mL} )} \times 100$$

3.4.3.3 Calculate the percent amount of Z loading as:

$$\frac{Determined sample Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated Z in the test sample to one decimal place (e.g. 96.5%).
3.5.2 Report the percent free Z in the test sample to one decimal place (e.g. 3.5%).

### Appendix K: Quantification Method for Zeaxanthin Levels from Milk by HPLC

### 1.0 Purpose

The purpose of this Standard Operating Procedure is to establish the sample preparation of Zeaxanthin encapsulated microparticles, extracted from milk and associated analytical methods for the quantitative determination of Zeaxanthin by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent
2.1.7 Whole milk or Zeaxanthin (free or microparticles) fortified milk

### 2.2 Reference Materials

| Name | Resource, Purity, and Others (Expiration Date) |
|---|---|
| Zeaxanthin | Shandong Tianyin Biotechnology Co., LTD, or IOSA |
| (powder)* | 70% Zeaxanthin, Exp Date: Unknown |
| Zeaxanthin | Southwest Research Institute, Zeaxanthin loading |
| microparticles | (%) and other ingredients (%), Exp Date: TBD |
| (powder)** | |

*Once the manufacturer packages are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Zeaxanthin extraction from milk by liquid-liquid extraction method
2.3.1 For regular milk, weigh certain mg of Z encapsulated microparticles and spike them into the milk
2.3.2 For Z microparticle fortified milk product, use the milk product directly
2.3.3 Vortex and sonicate the milk solutions containing Z microparticles
2.3.4 Immediately after the agitation, transfer 0.5 mL of the milk solution into 2 mL Eppendorf vial
2.3.5 Add 0.5 mL of Ethanol into the milk solution to make 1 mL volume (the volume can be increased to make better dissolution if necessary) and vortex to cause partial dissolution of L microparticles and to induce release of Z from milk contents and MP matrix.
2.3.6 Add 0.5 mL of dichloromethane into the suspension and vortex (the volume can be increased to make better dissolution and better separation of L into the DCM rich bottom layer, if necessary)
2.3.7 Centrifuge the tube at 10000 RPM for 2 min (the centrifuge speed and time can be adjusted to obtain better extraction).
   2.3.7.1 It will form three layers, two liquid layers (phase separation) and a solid layer of milk contents in between those two liquid layers.
   2.3.7.2 The bottom liquid layer (DCM rich layer) should have majority of orange color from Z.
2.3.8 Prepare 900 μL of DCM in HPLC vials
2.3.9 Take 100 μL of the bottom liquid layer from the centrifuged mixture sample using a pipet carefully not to interrupt the bottom solid pellet and top aqueous layer
2.3.10 Add it into the prepared 900 μL DCM in HPLC vial (1/10× dilution, Dilution factor 10). Be careful not to lose the solution by dripping from the pipet tip, due to the low surface tension of DCM rich solution
2.3.11 Cap the 1/10× diluted extract
2.3.12 Vortex the vial
2.3.13 Inject into HPLC following the HPLC method defined in the later section
2.3.14 At least 3 sample replicates and at least 3 neat condition controls (extracted the same way but matrix (milk) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Zeaxanthin powder
2.4.3 Suspend the powder in pure milk to make a suspension at 1.429 mg/mL for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: pure milk 1:1 mixture (at least 40 mL)
2.4.7 Prepare 10 empty 2 mL Eppendorf tubes for each Zeaxanthin
2.4.8 Fill 1 mL of EtOH: pure milk mixture into 9 of them (1 empty and 9 with 1 mL EtOH)
2.4.9 Vortex the Zeaxanthin stock suspension and immediately after the agitation, transfer 1.0 mL of the suspension into one empty tube and one of 9 tubes with
1.0 mL EtOH: pure milk mixture (making 1 mL as the same concentration and
2 mL of 1/2× dilution)
2.4.10 After agitation, take out 1 mL from 1/2× dilution and add into the next 1 mL pure EtOH: pure milk mixture making second 1/2× dilution. Continue this 1/2×serial dilutions into the next tube to make 10 concentration levels for each Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of Z into the DCM rich bottom layer, if necessary)
2.4.13 Centrifuge the tube at 10000 RPM for 2 min (the centrifuge speed and time can be adjusted to obtain better extraction).
   2.4.13.1 It will form three layers, two liquid layers (phase separation) and a solid layer of milk contents in between those two liquid layers.
   2.4.13.2 The bottom liquid layer (DCM rich layer) should have majority of orange color from L.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Zeaxanthin at 1.429 mg/mL in pure |
| | milk (freshly prepared*, vortexed, |
| | equivalent to 1.0 mg/mL pure |
| | Zeaxanthin) |
| | Z is not soluble and suspendable in |
| | milk. So, serial dilution to prepare |
| | for the calibration standards should |
| | be performed after adding EtOH (milk |
| | sample:EtOH 1:1) and diluted into |
| | EtOH:pure milk (1:1) mixture |
| Internal Standard Solution (IS) | N/A |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.2 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 ug/mL Zeaxanthin |
| | 0.195 ug/mL Zeaxanthin |
| Quality Control Samples for | N/A, to be included in the future |
| Zeaxanthin diluted from PS | |
| solutions | |

*All samples must be prepared freshly, due to instability.
** The Z range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

### 2.5 Equipments and Supplies

2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Zeaxanthin at 2.04
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Zeaxanthin) | 3 |
| Diluent Blank | 1 |
| Z calibration Std 1 to Std 10 | 1 |
| Diluent Blank | 1 |
| Unknown samples (Zeaxanthin/MPs extract from milk) | 1 |
| Diluent Blank | 1 |
| Z calibration Std 1 to Std 9 | 1 |
| Diluent Blank | 1 |
| System Suitability (100 μg/mL Zeaxanthin) | 3 |
| Diluent Blank | 1 |

### 3.4 Data Analysis

3.4.1 System Suitability
   3.4.1.1 The chromatographic profile of the 100 μg/mL Z Standards (or any other concentrations chosen as quality control purpose, determined using fresh Z) are comparable to the chromatogram determined using very fresh Z.
   3.4.1.2% RSD≤3 for the main peak areas of the six 100 μg/mL Z Standard (or any other concentrations chosen as quality control purpose, determined using fresh Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 10 levels×2 set=15 Stds, maximum 5 out of 20 are allowed to be excluded as outliers)
   3.4.2.3 At LLOQ (lowest limit of quantification), the signal/background ratio must be higher than 5.
   3.4.2.4 Standard curve must have an R²>0.99.
   3.4.2.5 The percent recovery of standard levels must be within 90% to 110% of its label claim.
3.4.3 Zeaxanthin, Zeaxanthin Concentrations in samples
   3.4.3.1 Using the standard curve values for Z, calculate the concentration of L using the following equation:

$$Z ( \frac{mg}{mL} ) = \frac{{A}_{Test} - y intercept}{slope} \times DF \times 0.001$$

Aₜₑₛₜ=Average main peak area of L
DF=dilution factor of the test sample.
0.001=convert μg/mL to mg/mL
3.4.3.2 Calculate the percent recovery of the test sample as:

$$\frac{Extracted Z concentration ( \frac{mg}{mL} )}{Neat condition control Z test sample ( \frac{mg}{mL} )} \times 100$$

3.4.3.3 Calculate the percent amount of Z loading as:

$$\frac{Determined sample Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated Z in the test sample to one decimal place (e.g. 96.5%).
3.5.2 Report the percent free Z in the test sample to one decimal place (e.g. 3.5%).

### Appendix L: Quantification Method for Zeaxanthin Levels from Probiotic Capsules by HPLC

### 1.0 Purpose

The purpose of this Standard Operating Procedure is to establish the sample preparation of Zeaxanthin encapsulated microparticles, extracted from probiotic capsule product with other ingredients and associated analytical methods for the quantitative determination of Zeaxanthin by HPLC

### 2.0 Materials and Solutions

### 2.1 Solutions

2.1.1 Water, HPLC grade: Sigma-Aldrich, Part #: 270733, or equivalent
2.1.2 Formic acid, reagent grade (>95%): Sigma-Aldrich, Part #: F0507, or equivalent or higher quality grade
2.1.3 Water with 0.1% formic acid: Sigma-Aldrich, Part #: 4.85085, or equivalent
2.1.4 Acetonitrile, HPLC grade: Sigma-Aldrich, Part #: 34851, or equivalent
2.1.5 Ethanol, 200 proof: Decon Labs, Part #: 22-032-601 (UN1170), or equivalent, or HPLC grade, Fisher Scientific, Part #: AC611050040, or equivalent
2.1.6 Dichloromethane, ACS reagent, Part #: 32222 (UN1593) or equivalent

### 2.2 Reference Materials

| Name | Resource, Purity, and Others (Expiration Date) |
|---|---|
| Zeaxanthin | Shandong Tianyin Biotechnology Co., LTD, |
| (powder)* | or IOSA |
| | 70% Zeaxanthin, Exp Date: Unknown |
| Zeaxanthin | Southwest Research Institute, Zeaxanthin loading |
| microparticles | (%), and other additives (%), Exp Date: Unknown |
| (powder)** | |
| Probiotics Capsule | Manufacturer: TBD |
| product containing | Zeaxanthin content (% or mg) Zeaxanthin content |
| Z microparticles | (% or mg), and other ingredients (% or mg), Exp |
| | Date: TBD |

*Once the manufacturer packages are opened, aliquot them in small quantity for a single use or short term use only (a few 100s mgs in glass vials), N₂ gas head them and vacuum seal them in aluminum sealing bag, then stored them in designated temperature until use. When used, they should be equilibrated to room temperature at least for 30 minutes before opening aliquots.

2.3 Zeaxanthin extraction from probiotics capsule content mixture by liquid-liquid extraction method
2.3.1 Open capsule and collect powder contents in a separate vial
2.3.2 Weigh certain mg of the collected capsule content powder
2.3.3 Suspend the powder in DI water to make a suspension at 20 mg/mL concentration
2.3.4 Vortex and sonicate the suspension
2.3.5 Immediately after the agitation, transfer 0.5 mL of the suspension into 2 mL Eppendorf vial
2.3.6 Add 0.5 mL of Ethanol into the suspension and vortex to form partially dissolved suspension to make 1 mL volume (the volume can be increased to make better dissolution if necessary)
2.3.7 Add 0.5 mL of dichloromethane into the suspension and vortex (the volume can be increased to make better dissolution and better separation of Z into the DCM rich bottom layer, if necessary)
2.3.8 Centrifuge the tube at 10000 RPM for 2 min (the centrifuge speed and time can be adjusted to obtain better extraction).
   2.3.8.1 It will form two liquid layers (phase separation) and pellet of solid in the bottom of the tube.
   2.3.8.2 The bottom liquid layer (DCM rich layer) should have majority of orange color from Z.
2.3.9 Prepare 900 μL of DCM in HPLC vials
2.3.10 Take 100 μL of the bottom liquid layer from the centrifuged mixture sample using a pipet carefully not to interrupt the bottom solid pellet and top aqueous layer
2.3.11 Add it into the prepared 900 μL DCM in HPLC vial (1/10× dilution, Dilution factor 10). Be careful not to lose the solution by dripping from the pipet tip, due to the low surface tension of DCM rich solution
2.3.12 Cap the 1/10× diluted extract
2.3.13 Vortex the vial
2.3.14 Inject into HPLC following the HPLC method defined in the later section
2.3.15 At least 3 sample replicates and at least 3 neat condition controls (extracted the same way but matrix (capsule content suspension) was substituted with DI water) should be prepared for recovery determination

### 2.4 Primary Stock Solutions, Spiking Stock Solutions, Calibration Standards/QC Samples

2.4.1 All calibration standards/QC samples are treated as the same way as the extraction method stated in Section 2.3
2.4.2 Weigh certain mg (at least 3 mg) of the fresh (or properly stored) Zeaxanthin powder
2.4.3 Suspend the powder in DI water to make a suspension at 1.429 mg/mL for Zeaxanthin (equivalent to 1.0 mg/mL of pure Zeaxanthin) concentrations as primary stock solutions.
2.4.4 Vortex and sonicate the stock suspension
2.4.5 Add the same volume of Ethanol into each stock suspension and vortex to form partially dissolved suspension
2.4.6 Prepare EtOH: DI water 1:1 mixture (at least 40 mL)
2.4.7 Prepare 10 empty 2 mL Eppendorf tubes for each
2.4.8 Fill 1 mL of EtOH: DI water mixture into 9 of them (1 empty and 9 with 1 mL EtOH)
2.4.9 Vortex the Zeaxanthin stock suspension and immediately after the agitation, transfer 1.0 mL of the suspension into one empty tube and one of 9 tubes with
1.0 mL EtOH: DI water mixture (making 1 mL as the same concentration and
2 mL of 1/2× dilution)
2.4.10 After agitation, take out 1 mL from 1/2× dilution and add into the next 1 mL pure EtOH: DI water mixture making second 1/2× dilution. Continue this 1/2×serial dilutions into the next tube to make 10 concentration levels for each Zeaxanthin
2.4.11 Take out 1 mL out of the last tube (the lowest concentration having 2 mL total) and dispose the 1 mL solution (making all 10 tubes have 1 mL each)
2.4.12 Add 0.5 mL of dichloromethane into each tube and vortex (the volume can be increased to make better dissolution and better separation of L into the DCM rich bottom layer, if necessary)
2.4.13 Centrifuge the tube at 10000 RPM for 2 min (the centrifuge speed and time can be adjusted to obtain better extraction).
   2.4.13.1 It will form two liquid layers (phase separation) and pellet of solid in the bottom of the tube.
   2.4.13.2 The bottom liquid layer (DCM rich layer) should have majority of orange color from Z.
2.4.14 Prepare 900 μL of DCM in HPLC vials
2.4.15 Take 100 μL of the bottom liquid layer from the centrifuged calibration standard sample using a pipet carefully not to interrupt the bottom solid pellet and top aqueous layer
2.4.16 Add it into the prepared 900 μL DCM in HPLC vial (1/10× dilution, Dilution factor 10). Be careful not to lose the solution by dripping from the pipet tip, due to the low surface tension of DCM rich solution
2.4.17 Cap the 1/10× diluted extract
2.4.18 Vortex the vial
2.4.19 Inject into HPLC following the HPLC method defined in the later section

| Solutions | Concentration (range) |
|---|---|
| Primary Stock Solutions (PS) | Zeaxanthin at 1.429 mg/mL in DI |
| | water (freshly prepared*, vortexed, |
| | equivalent to 1.0 mg/mL pure |
| | Zeaxanthin) |
| | Z is not soluble and suspendable in |
| | water. So, serial dilution to prepare |
| | for the calibration standards should |
| | be performed after adding EtOH |
| | (sample:EtOH 1:1) and diluted into |
| | water:EtOH (1:1) mixture |
| Internal Standard Solution (IS) | N/A |
| Calibration Standard Samples | 100 μg/mL Zeaxanthin |
| for Zeaxanthin, diluted from | 50 μg/mL Zeaxanthin |
| PS solutions (Note: they are | 25 μg/mL Zeaxanthin |
| nominal concentration by input | 12.5 μg/mL Zeaxanthin |
| without considering dilutions | 6.2 μg/mL Zeaxanthin |
| during extraction process, not | 3.125 μg/mL Zeaxanthin |
| actual concentration) ** | 1.563 μg/mL Zeaxanthin |
| | 0.781 μg/mL Zeaxanthin |
| | 0.391 ug/mL Zeaxanthin |
| | 0.195 ug/mL Zeaxanthin |
| Quality Control Samples for | N/A, to be included in the future |
| Zeaxanthin and/or Zeaxanthin, | |
| diluted from PS solutions | |

*All samples must be prepared freshly, due to instability.
** The L range are subject to be changed depending on the instrument's sensitivity and maximum detection range.

Below is an example of how to prepare calibration standards from stock solutions

| | | Vol. | | | | | | | |
|---|---|---|---|---|---|---|---|---|---|
| | Spiking | Spiking | Vol. | | | Vol. | | | |
| | Solution | solution | EtOH:Water | Total | | DCM | | | Nominal |
| Spiking | Conc. | to add | mixture | Volume | | to add | Dilution | | Std conc. |
| solution ID | (μg/mL) | (μL) | (μL) | (μL) ^{b} | Stock ID | (uL) | in DCM | STD ID | (μg/mL)** |
| Zeaxanthin | 1000 (1429)* | | | 1000 | Z stock 10 | 500 | 1/10x | Z STD 10 | 100 |
| Primary | | | | | | | | | |
| stock | | | | | | | | | |
| Z Stock 10 | 1000 | 1000 | 1000 | | Z Stock 9 | 500 | 1/10x | Z STD 9 | 50 |
| Z Stock 9 | 500 | 1000 | 1000 | | Z Stock 8 | 500 | 1/10x | Z STD 8 | 25 |
| Z Stock 8 | 250 | 1000 | 1000 | | Z Stock 7 | 500 | 1/10x | Z STD 7 | 12.5 |
| Z Stock 7 | 125 | 1000 | 1000 | | Z Stock 6 | 500 | 1/10x | Z STD 6 | 6.25 |
| Z Stock 6 | 62.5 | 1000 | 1000 | | Z Stock 5 | 500 | 1/10x | Z STD 5 | 3.125 |
| Z Stock 5 | 31.25 | 1000 | 1000 | | Z Stock 4 | 500 | 1/10x | Z STD 4 | 1.563 |
| Z Stock 4 | 15.63 | 1000 | 1000 | | Z Stock 3 | 500 | 1/10x | Z STD 3 | 0.781 |
| Z Stock 3 | 7.81 | 1000 | 1000 | | Z Stock 2 | 500 | 1/10x | Z STD 2 | 0.391 |
| Z Stock 2 | 3.91 | 1000 | 1000 | | Z Stock 1 | 500 | 1/10x | Z STD 1 | 0.195 |
| | | | 1000 | | Blank | 500 | 1/10x | | |

*Zeaxanthin PS concentration including impurity
**These concentrations are nominal concentrations not actual concentration

2.5 Equipment and supplies
2.5.1 HPLC with UV/VIS detector
2.5.2 Phenomenex Gemini® C18 column, 3 μm, 100×3 mm, Part #: 00D-4439Y0, or any equivalent C18 columns
2.5.3 Borosilicate glass bottles: 500 mL, 1 L, or 2 L capacity
2.5.4 Borosilicate glass vials, 4 mL, 7 mL, and 20 mL capacity
2.5.5 Positive displacement pipettes capable of measuring volumes between 20 μL to 25 mL
2.5.6 2 mL glass HPLC vials with PTFE/Silicone septa vial caps
2.5.7 Analytical balance
2.5.8 Vortexer

### 3.0 HPLC Method

### 3.1 Mobile Phase Preparation

3.1.1 Mobile Phase A (Aqueous):
   3.1.1.1 Option 1: pure HPLC grade water as stated in 2.1.2
   3.1.1.2 Option 2:
      3.1.1.2.1 In a 1 L glass bottle, add 1000 mL of HPLC grade water (2.1.2)
      3.1.1.2.2 Add 1 mL of formic acid (2.1.3), Cap and mix solution thoroughly
      3.1.1.2.3 Store at room temperature. Use by date is 2 weeks from preparation
   3.1.1.3 Option 3: Water with 0.1% formic acid as stated in 2.1.4
3.1.2 Mobile Phase B (Organic):
   3.1.2.1 Option 1: Pure acetonitrile as stated in 2.1.5
   3.1.2.2 Option 2: Acetonitrile with 0.1% formic acid as stated in 2.1.6
3.1.3 Diluent Blank: Pure DCM as stated in 2.1.1

### 3.2 HPLC Conditions

LC system: Agilent 1260 or 1290, or equivalent
Column: Phenomenex Gemini® C18 column, 3 μm, 100×3 mm
Detector: Agilent MWD detector (G7165A) or DAD detector (G4212A)
Detection wavelength: 450 nm, slit width: 4 nm
Column temperature: 20° C.
Autosampler temperature: 2-8° C.
Data system: Agilent CDS
Mobile phase A: HPLC grade water (or 0.1% formic acid in water)
Mobile phase B: Acetonitrile (or Acetonitrile with 0.1% formic acid)
Gradient: Not necessary (Isocratic mode)

| Step | Time (min) | A (%) | B (%) |
|---|---|---|---|
| 0 | 0 | 1 | 99 |
| 1 | 3.6 | 1 | 99 |

Mobile phase flow rate: 1.0 mL/min
Injection volume: 2 μL
Run time: 3.6 minutes
Retention times: Zeaxanthin at 2.04 minute
Needle Washing Solvent: TBD (tentatively, water: Acetonitrile 1:1 v/v mix)

### 3.3 HPLC Sequence

3.3.1 Note: Equilibrate HPLC and Column for a minimum 15 minute before injecting samples

| Vial Description | Injections |
|---|---|
| Diluent Blank | 1 |
| System Suitability (100 μg/mL Zeaxanthin) | 3 |
| Diluent Blank | 1 |
| Z calibration Std 1 to Std 10 | 1 |
| Diluent Blank | 1 |
| Unknown samples (Zeaxanthin/MPs extract from capsule) | 1 |
| Diluent Blank | 1 |
| Z calibration Std 1 to Std 9 | 1 |
| Diluent Blank | 1 |
| System Suitability (100 μg/mL Zeaxanthin) | 3 |
| Diluent Blank | 1 |

### 3.4 Data Analysis

3.4.1 System Suitability
   3.4.1.1 The chromatographic profile of the 100 μg/mL Z Standards (or any other concentrations chosen as quality control purpose, determined using fresh Z) are comparable to the chromatogram determined using very fresh Z.
   3.4.1.2% RSD≤3 for the main peak areas of the six 100 μg/mL Z Standard (or any other concentrations chosen as quality control purpose, determined using fresh Z) injections.
3.4.2 Calibration Standards
   3.4.2.1 Using the average peak area from the two injections of each standard level, generate a standard curve and determine the coefficient of determination (R²), slope, and y-intercept.
   3.4.2.2 For each L standards, at least 75% of standards must be included in calculation of 3.4.2.1. (e.g. 9 levels×2 set=18 Stds, maximum 4 out of 18 are allowed to be excluded as outliers)
   3.4.2.3 At LLOQ (lowest limit of quantification), the signal/background ratio must be higher than 5.
   3.4.2.4 Standard curve must have an R²≥0.99.
   3.4.2.5 The percent recovery of standard levels must be within 90% to 110% of its label claim.
3.4.3 Zeaxanthin, Zeaxanthin Concentrations in samples
   3.4.3.1 Using the standard curve values for Z, calculate the concentration of Z using the following equation:

$$Z ( \frac{mg}{mL} ) = \frac{{A}_{Test} - y intercept}{slope} \times DF \times 0.001$$

Aₜₑₛₜ=Average main peak area of Z
DF=dilution factor of the test sample.
0.001=convert μg/mL to mg/mL
3.4.3.2 Calculate the percent recovery of the test sample as:

$$\frac{Extracted Z concentration ( \frac{mg}{mL} )}{Neat condition control Z test sample ( \frac{mg}{mL} )} \times 100$$

3.4.3.3 Calculate the percent amount of Z loading as:

$$\frac{Determined sample Z concentration ( \frac{mg}{mL} )}{MP input concentration ( \frac{mg}{mL} )} \times 100$$

3.4.3.4 The percent recovery of each test sample must be within 85% to 110% of its neat condition control.
3.4.3.5 The % RSD (coefficient of variation) among 3 replicates should be less than 5%.

### 3.5 Reporting

3.5.1 Report the percent encapsulated Z in the test sample to one decimal place (e.g. 96.5%).
3.5.2 Report the percent free Z in the test sample to one decimal place (e.g. 3.5%).

1. A particle preparation comprising a payload component, a polymer component, and a residual solvent content lower than a predetermined amount, a microparticle, a first excipient component, and a second excipient component, wherein:
(i) the payload component comprises a nutraceutical comprising at least one antioxidant, at least one macronutrient, at least one micronutrient, at least one mineral, at least one prebiotic, at least one probiotic, at least one vitamin, or any combination thereof;
(ii) the polymer component comprises a pH-responsive polymer component comprising a methacrylate, a polygalactomannan, a polysaccharide, or any combination thereof;
(iii) the predetermined amount is in a range-from 1 ppm to 5000 ppm;
(iv) microparticle loading is from 45% to 90%;
(v) first excipient component loading is from 10% to 50%;
(vi) second excipient component loading is from 0% to 45%, and
wherein the particle preparation is formed by adding at least one of the first excipient component and the second excipient component to the microparticle loading during milling.

2-9. (canceled)

10. The particle preparation of claim 1, characterized as releasing at least 80%, at least 90%, and/or at least 95% of the payload component within 5 minutes when included in an environment having a pH of less than 5.

11. The particle preparation of claim 1, characterized as releasing at most 20%, about at most 15%, at most 10%, and/or at most 5% of the payload component after soaking the particle preparation for at least 2 hours in an environment having a pH of 7 and a temperature within a range of 25° C. to 100° C.

12-16. (canceled)

17. The particle preparation of claim 1, wherein the pH-responsive polymer component is structured to allow controlled release of the payload component when exposed to an environment with a pH of 5.0 or lower,
wherein the pH-responsive polymer component is structured to be stable when exposed to an environment with a pH of 6.0 or higher, and
wherein the pH-responsive polymer component is stable when exposed to temperatures in a range from 1° C. to 100° C.

18. (canceled)

19. The particle preparation of claim 1,
wherein the first excipient component comprises at least one of soy lecithin, sunflower lecithin, maltodextrin 40, dryflo, and fructose, and
wherein the second excipient component comprises at least one of soy lecithin and maltodextrin 40.

20. The particle preparation of claim 19, further comprising a third excipient component loading in a range from 0% to 5%,
wherein the third excipient comprises dryflo.

21. The particle preparation of claim 1, wherein the microparticle loading is from 70% to 90%,
wherein the first excipient component loading is from 10% to 30%, and
wherein the second excipient component loading is from 0% to 15%.

22-87. (canceled)

88. The particle preparation of claim 1, wherein the particle preparation maintains water activity of less than 0.3, less than 0.2, and/or less than 0.1 in a sealed storage environment for up to 6 months at −20 C, 4 C, 25 C, 30 C and 75% relative humidity (RH), and/or at 40 C and 75% RH.

89. The particle preparation of claim 1, wherein the particle preparation is chemically stable in an unsealed storage environment for up to 6 months at 25 C and 75% RH.

90. The particle preparation of claim 1, wherein the particle preparation is stable in direct light exposure for up to 72 hours at 37 C.

91-98. (canceled)
